(19) United States
(12) Patent Application Publication
(10) Pub. No.: US 2024/0379240 A1
(43) Pub. Date: Nov. 14, 2024

(54) METHODS OF PREDICTING PHENOME-WIDE POLYGENIC RISK SCORES USING MULTI-TASK LEARNING

(71) Applicant: THE BOARD OF REGENTS OF THE UNIVERSITY OF OKLAHOMA, Norman, OK (US)

(72) Inventors: Chongle PAN, Norman, OK (US); Adrien F. BADRE, Oklahoma City, OK (US)

(73) Assignee: THE BOARD OF REGENTS OF THE UNIVERSITY OF OKLAHOMA, Norman, OK (US)

(21) Appl. No.: 18/631,773

(22) Filed: Apr. 10, 2024

Related U.S. Application Data
(60) Provisional application No. 63/458,507, filed on Apr. 11, 2023.

(51) Int. Cl. G16H 50/30; G06N 3/08

(52) U.S. Cl. CPC G16H 50/30; G06N 3/08

(57) ABSTRACT

Provided herein are methods of using multi-task learning (MTL) neural network architecture for predicting many disease traits of an individual from their whole genome. The model used a shared latent genomic representation, and each trait was predicted from the shared representation via a task-specific hidden layer. The MTL models achieved higher predictive performance than single-task learning (STL) models for both cancer and non-cancer diseases.

### REFERENCE TO RELATED APPLICATIONS

The present application claims the priority benefit of U.S. provisional application No. 63/458,507, filed Apr. 11, 2023, the entire contents of which are incorporated herein by reference.

### STATEMENT REGARDING FEDERALLY SPONSORED RESEARCH OR DEVELOPMENT

This invention was made with government support under grant number R01AT011618 awarded by the National Institutes of Health. The government has certain rights in the invention.

### BACKGROUND

Machine Learning is a subfield of Artificial Intelligence (AI) where machines learn a task without being explicitly programmed for it, according to Arthur Samuel (El Naqa and Murphy, 2015). In machine learning, models are created using algorithms that can learn from data and make predictions or decisions based on that data. The models are trained on historical data, and the goal is to make accurate predictions or decisions about new, unseen data. The two types of machine learning are supervised learning and unsupervised learning.

Supervised learning takes a dataset with features and labels as input and learns their relationships. More formally, let define D={(x₁,y₁), . . . , (xᵢ, yᵢ), . . . , (x_{N},y_{N})} a dataset of size N. xᵢ is a feature vector of dimension dₓ, and yᵢ its label. Let X be a matrix of feature vectors and Y a vector of labels, continuous or discrete. It is assumed that a relationship between X and Y exists. Let F be a model that has the capacity to learn the conditional probability P (Y|X) and G a model that can learn the joint distribution P (Y,X). In a supervised learning setup, the goal of F is to satisfy best a penalty function that models the bias/variance trade-off, while G empirically seeks the function that best fits the training data. The more the model becomes complex during its training phase, the more its bias decreases while the variance increases. The optimal point of learning is when the model variance and bias are the lowest because this is where the model total error reached the lowest value. Accurate learning of the relationships is measured by a loss function:

$$Losstotal = {\sum }_{i = 0}^{n} Li ( yi , M ( xi ) )$$

where M is the model and M (xᵢ) is the score predicted by the model.

Unsupervised learning takes as input a dataset D={(x₁), . . . , (xᵢ), . . . , (x_{N})} without any label. Unlike supervised learning, where the algorithm is guided to represent a specific relationship between X and Y, the unsupervised learning algorithm needs to figure out by itself what relationship needs to make inside the dataset and create its own labels.

Supervised learning problems are divided into two categories: the regression tasks and the clarification tasks. Regression tasks can be defined as the process of finding the relationship between X and Y where Y is continuous. A classification task, however, is a process of finding the relationship between X and Y where Y is discrete.

The concept of interpretability in mathematics is not well defined. According to (Miller, 2019), interpretability can be defined as the extent to which a human can understand the reasoning behind a decision made by a model. Another definition is the extent to which a human can accurately predict the model's output. The more interpretable a machine learning model is, the easier it becomes for humans to comprehend its predictions or decisions. A model can be considered more interpretable if its decisions are easier for humans to understand. The terms interpretable and explainable are used interchangeably in this context. As used herein, interpretable machine learning refers to gaining meaningful insights from a machine learning model, whether the relationships are present in the data or learned by the model.

There are several types of supervised machine learning algorithms, including linear regression, logistic regression, and deep learning.

In linear regression, for a regression task,let's define a dataset D={(x₁, y₁), . . . , (xᵢ, yᵢ), . . . , (x_{N}, y_{N})}, as previously defined. The labels Y are continuous, and the dataset carries the following assumptions:
(1) Features in X are independent from each other,
(2) There is a linear relationship between X and Y,
(3) Homoscedasticity: The variance of residual is the same for any value of X, and
(4) For any fixed value of X, the mean of Y is normally distributed.

The relationship between the d features of x; and the response y; are modeled as follow:

$${y}_{i} = {β}_{d} {x}_{i}^{d} + {β}_{d - 1} {x}_{i}^{d - 1} + … + {β}_{1} {x}_{i}^{1} + {β}_{0} + {ϵ}_{i} \qquad ( 1.1 )$$

where the βᵢs represents the weights. There are d+1 weights: one for each dimension, and β₀ is the bias. ∈ᵢ is the error term. The Mean Square Error (MSE) is a common loss function that models the bias/variance trade-off. It is derived as:

$$MSE = \frac{1}{N} {\sum }_{i = 0}^{N} {( {y}_{i} - M ( {x}_{i} ) )}^{2} \qquad ( 1.2 )$$

where M is our linear regression model. The objective function is:

$$arg {min}_{M} ( MSE ) = \frac{1}{N} {\sum }_{i = 0}^{N} {( {y}_{i} - {M}^{*} ( {x}_{i} ) )}^{2} \qquad ( 1.3 )$$

Where M* is the optimal model M that minimizes the best MSE.

In logistic regression, for a classification task, a dataset D={(x₁,y₁), . . . , (xᵢ, yᵢ), . . . , (x_{N}, Y_{N})}, is defined. The labels Y are binary, and the dataset carries the following assumptions:
(1) Features in X are independent from each other,
(2) There is a linear relationship between X and the response logit, and
(3) For any fixed value of X, the Y is normally distributed.

The logistic regression model can be derived as follows:

$$log it = log \frac{p}{1 - p} = {β}_{d} {x}_{i}^{d} + {β}_{d - 1} {x}_{i}^{d - 1} + … + {β}_{1} {x}_{i}^{1} + {β}_{0} + {ϵ}_{i} \\ p = \frac{1}{1 + {e}^{- logit}} \qquad ( 1.4 )$$

where p is the probability of a positive outcome. The function

$$f ( x ) = \frac{1}{1 + {e}^{- x}}$$

is called the sigmoid function and was introduced by Pierre Francois Verhulst in the 19th century. The log-loss is a common loss function that models how close the prediction probability is to the actual binary values. It is derived as:

$${log}_{l} oss = \frac{1}{N} {\sum }_{i = 0}^{N} {y}_{i} * log M ( {x}_{i} ) + ( 1 - {y}_{i} ) log 1 - M ( {x}_{i} ) ) \qquad ( 1.5 )$$

where M is our logistic regression model. The objective function is:

$$arg {min}_{M} ( {log}_{l} oss ) = \frac{1}{N} {\sum }_{i = 0}^{N} {y}_{i} log ( {M}^{*} ( {x}_{i} ) ) + ( 1 - {y}_{i} ) log ( 1 - {M}^{*} ( {x}_{i} ) ) \qquad ( 1.6 )$$

where M* is the optimal model M that minimizes the best the log-loss.

In Deep Learning, the supervised classification task can be extended to Artificial Neural Networks (ANN) enabling the concept of Multi-Task Learning (MTL). ANNs are complex statistical models inspired by biological neural networks. They are composed of neurons designed on the model of biological neurons. The structure of the artificial neuron is very similar to the biological neuron. They both share a structure to receive an input signal and, based on its strength, decide to produce a corresponding output signal. The artificial neurons take a signal as input from the preceding neurons' weighted outputs and get activated, based on the sum of these outputs. The function that decides whether the neuron gets activated is called the activation function. Like a biological neuron, if the strength of the input signal is strong enough, the neuron is activated and delivers a signal to the rest of the network it is connected to. If the connection between two neurons is deemed of importance, then, as with biological networks, the connection is reinforced. This gave birth to the perceptron by Franck Rosenblatt in 1958 (Rosenblatt, 1958). Artificial neural networks are constructed using layers of neurons. One layer contains several neurons, takes input from a preceding layer, and connects its outputs to another layer. When the neurons of each network layer are connected to all the neurons of the preceding layer and the next layer, it is called a fully connected layer (FIG. 1). A network constructed with such layers is called a Feed Forward Neural Network (FFNN).

In a neuron, the strength of the input signal depends on the weights attributed to each connection. A neuron has one weight wn.i./for each input connection. The output of a neuron n on layer l, given d neurons on the previous layer is given by:

$$outputn , l = f ( {\sum }_{i = 1}^{d} wn , i , l - 1 outputi , l - 1 ) \qquad ( 1.7 )$$

where f is the activation function of the neuron n.

This function can be used to activate the output neuron and map a real number to a probability. Formally, for x∈R, sigmoid (x) ∈[0,1]. Another activation function is ReLU (Fukushima, 1975). The ReLU function is defined as follows:

$$ReLU ( x ) = \{ \begin{matrix}x , & if x \geq 0 \\ 0 , & otherwise\end{matrix} \qquad ( 1.8 )$$

This function is mainly used for neurons on hidden layers (layers that are between the input layer and the output layer) and introduces non-linearity to the network. However, the dying neuron is a drawback inherent to ReLU (Lu et al., 2019). Leaky ReLU was proposed to solve this issue (Maas et al., 2013). It allows the negative signals to be output with a small coefficient α. Formally it is defined as (Eqn. 1.9):

$$Leaky\_ReLU ( x ) = \{ \begin{matrix}x , & if x \geq 0 \\ - α x , & otherwise\end{matrix}$$

Training a FFNN for a supervised learning task requires the same setup as the linear regression for regression tasks or the logistic regression for classification tasks. Define again a dataset D={(x₁,y₁), . . . , (xᵢ,y;), . . . , (x_{N}, Y_{N})} a dataset of size N. xᵢ is a feature vector of dimension dₓ, and yᵢ its label. Let X be a matrix of feature vectors and Y a vector of continuous labels. The neural network F takes X as input and must learn the relationship between X and Y. The training goal is to minimize the loss function L. Hence, the network must go through learning steps. At each learning step, a forward pass is realized. The network makes predictions using the input data. Then, the loss is measured to quantify the error. Finally, this error is backpropagated to the network that adapts its weights to minimize this error (Rumelhart et al., 1986). The algorithm is detailed in Algorithm 1.2.1. The Adam optimizer (Kingma and Ba, 2014a) version of the stochastic gradient update can be used to introduce an adaptive estimate of lower-order moments and results in a faster and better gradient update algorithm.

Algorithm 1.2.1 Error backpropagation to the iₜₕ weight on neuron n, on
layer l, w_{n,i,l} in a feed-forward neural network, at learning step t.

(1) ∀(xᵢ, yᵢ) ∈ D, propagate the input xᵢ through the network to compute
the outputs Y ^{p}, the vector containing all the yᵢ^{p}.
(2) Compute the loss L(Y^{p}, Y), with Y the vector containing all the yᵢ
(3) For each weight W_{n,i,l} compute
${w}_{n , i , l}^{t + 1} = {w}_{n , i , l}^{t} - α \frac{\partial ℒ ( {Y}^{p} , Y )}{\partial {w}_{n , i , l}} \qquad ( 1.1 )$
with a referring to the learning rate, and t the learning step.

Deep Neural Networks (DNN) are an ANN type that has more than 2 hidden layers. DNNs have been widely studied in the past years. They have met tremendous success in a vast number of different tasks, including audio and speech processing, visual data processing, and natural language processing (NLP) (Adeel et al., 2020; Tian et al., 2020; Young et al., 2018; Koppe et al., 2021). Among them, Convolutional Neural Networks (CNN) (LeCun et al., 1998) have been part of these tremendous successes. CNNs perform pointwise multiplication of the input features with a moving filter across the feature space. Define each unit computation position the offset t. The convolution operation for 1d datasets can be computed as follows:

$$Conv ( X , W , τ ) = {\sum }_{i = - ∞}^{+ ∞} {x}_{i} {w}_{τ - i} \qquad ( 1.11 )$$

where X has d features, W is the kernel function (or weights of the neural network), xᵢ is a unique feature vector, and wᵢ is a weight in the filter. Multiple filters can be applied at each feature to decompose the signal into higher-order features. After the convolution operator is applied, the extracted feature space can be condensed using the pooling operator. Several blocks of convolution and pooling can be added together to condense the extracted feature. Then, those features can be flattened and passed to a FFNN to make the final prediction.

Multi-task Learning (Caruana, 1998) is a transfer learning method that enhances one task's performance by utilizing the information gained from related tasks. It accomplishes this by simultaneously training on multiple tasks while sharing a common representation, allowing what is learned from one task to improve the learning of other tasks. Multi-task learning was used with success in several different problems (Zhang and Yang, 2018), such as natural language processing (Collobert and Weston, 2008), speech recognition (Deng et al., 2013) or computer vision (Girshick, 2015). Formally, define a dataset D={(x₁,y₁), . . . , (xᵢ, yᵢ), . . . , (x_{N},Y_{N})} a dataset of size N. xᵢ is a feature vector of dimension dₓ, and yᵢ its vector labels of dimension dy. Each dimension of yᵢ refers to the label of xᵢ for task Tᵢ. X is a matrix of feature vectors and Y a matrix of labels, Y_{Ti} being the label vector for task Tᵢ. The network F takes X as input and must learn the relationship between X and Y. The network goal is to minimize the loss function L that is defined as follows:

$$Losstotal = {\sum }_{i = 0}^{n} {w}_{{T}_{i}} Li ( {Y}_{{T}_{i}} , {M ( X )}_{{T}_{i}} )$$

with wᵢ being the contribution weight of task Tᵢ to the global loss, and M(X)_{Ti}, the model output for task Tᵢ. In an example of a CNN for object classification with 1D signal, the signal is processed through several blocks of convolution and pooling, then the features are flattened to be processed by a FFNN for the final classification prediction.

Genomics encompasses the study of all genes in the genome, the interactions among genes, the genetic mutations, and the effects of genetic variants on human traits, known as phenotype. Mutations in an individual's genome can lead to dramatic changes in their phenotypes. A Single Nucleotide Polymorphism (SNP) is a DNA sequence variation that occurs when a single nucleotide (adenine, thymine, cytosine, guanine) at a particular locus in the genome sequence is altered and the particular alteration is present in at least 1% of the population. Different methods can be used to identify SNPs, such as dynamic allele-specific hybridization (Jobs, 2001), molecular beacons (Abravaya et al., 2003), and SNP microarrays (Steemers and Gunderson1, 2005; Thissen et al., 2019). SNP microarrays were notably used to sequence SNPs for the Oncorarray consortium (Amos et al., 2017), UKBiobank genomics data (Bycroft et al., 2018), and the 1000 Genome project data (Consortium et al., 2015). Sequenced personal genomes are compared with a reference genome that contains the most common variants at each locus within the population. Therefore, a dataset is created where each position can take 3 different values, as illustrated in Table 1. Table 1 shows an example of mapping the variation in SNPs at nine different loci, and how the data are represented. SNP5 is a locus having a mutation on the paternal side (A>C). SNP4 shows a locus having mutations (G>A) on both the paternal and maternal sides, wherein there is no distinction between the unique mutation on the maternal and paternal chromosomes.

For an individual i at position j, we have:

$${SNP}_{i , j} = \{ \begin{matrix}0 , & \begin{matrix}if both maternal and paternal \\ base pair at position j mutated\end{matrix} \\ 1 , & if only one mutated \\ 2 , & if no mutation\end{matrix} \qquad ( 1.13 )$$

TABLE 1
SNP mapping and individual data representation

| | SNP1 | SNP2 | SNP3 | SNP4 | SNP5 | SNP6 | SNP7 | SNP8 | SNP9 . . . |
|---|---|---|---|---|---|---|---|---|---|
| Reference | A | T | C | G | A | A | A | C | T |
| Paternal | A | T | C | A | C | A | A | C | T |
| Maternal | A | T | C | A | A | A | A | C | T |
| Individual | 2 | 2 | 2 | 0 | 1 | 2 | 2 | 2 | 2 |

Genome-Wide Association Studies (GWAS) map SNP arrays to a trait to unveil the associations of variants with this particular trait. GWAS are generally conducted on a population sourced from a biobank, such as the UKBiobank (Bycroft et al., 2018), or study cohorts for specific diseases (Mailman et al., 2007). Human subjects are recruited on a volunteer-based system where they are asked to transmit their medical history. In some cases, such as UKBiobank, they are followed up throughout their life for potential additional traits developing with age.

Several data processing methods can be used to process the data, such as the minor allele frequency criteria, the Hardy-Weinberg equilibrium, or linkage disequilibrium. Hardly-Weinberg equilibrium relates to the principle that genetic variations stay the same from one generation to another. In this case, chi-square tests 1.14 are applied between an expected genetic population versus the current actual population. The test is formulated as follows:

$${χ}^{2} = {\sum }_{i} \frac{{( {O}_{i} - {E}_{i} )}^{2}}{{E}_{i}} \qquad ( 1.14 )$$

Eᵢ being the expected value at sample i and O the observed value at sample i.

If the test indicates a statistical difference between the expected population and the observed population, then the observed SNP genetic structure is in disequilibrium. Disequilibrium can indicate a significant amount of mutation rate, or non-random mating for example. Linkage disequilibrium refers to the non-random correlation between SNPs. Minor allele frequency refers to the frequency of the recessive allele for on position among the population. PLINK is the most popular software to assess those principles and manage this type of genomic data (Purcell et al., 2007).

Ancestry is also an important feature to consider because it may introduce bias in detecting variants that can lead to false positive variants over a population (Marchini et al., 2004; Novembre et al., 2008; Lawson et al., 2020). Simple linear models may struggle to separate sub-populations effectively.

Statistical models have been widely used to model the relationships between SNPs and traits. Association analysis can be conducted using logistic regression between each SNP individually and the trait of interest. For each association, the p-value is calculated, and adjusted for multiple comparison using the Bonferroni level of significance (Bonferroni, 1935). The adjusted p-values are then commonly used to filter out significant SNPs from the non-important ones. Polygenic risk scores can be derived using the additive effect of the SNPs. Linear regression models, such as Best Linear Unbiased Prediction (BLUP) (Henderson, 1975), consider the additive effects of SNPs to determine the relative importance of those SNPs. The genetic effect of SNPs is also associated with non-fixed effects, such as weight and environmental or behavioral factors. The model is structured as follows:

$$Y = W α + {X}_{s} {β}_{s} + g + e \\ g \sim N ( 0 , {σ}_{a}^{2} ) \\ e \sim N ( 0 , {σ}_{e}^{2} ) \qquad ( 1.15 )$$

whereσₐ² represents the genetic variation, σ_{c}^{2the} residual variance for non-fixed effects, W is the covariates matrix for non-fixed effect, α its weight vector, Xₛ contains the SNPs matrix, and βs the SNPs weights (Uffelmann et al., 2021).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The accompanying drawings illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1 shows an example of a feed-forward neural network. Each neuron is connected to all the neurons of the previous layer and the neurons of the next layer.

FIG. 2 shows a schematic of a computational workflow of predictive genomics. The DRIVE dataset was randomly split into the training set, the validation set, and the test set. Only the training set was used for association analysis, which generated the p-values for the selection of SNPs as input features. The training data was then used to train machine learning models and statistical models. The validation set was used to select the best hyperparameters for each model based on the validation AUC score. Finally, the test set was used for performance benchmarking and model interpretation.

FIG. 3 shows results of SNP filtering using a Manhattan plot from the association analysis. Each point represents a SNP with its p-value in the log 10 scale on the y-axis and its position in a chromosome on the x-axis. The x-axis is labeled with the chromosome numbers. Chromosome 23 represents the X chromosome. Chromosomes 24 and 25 represent the pseudoautosomal region and non-pseudoautosomal region of the Y chromosome, respectively. Chromosome 26 designates the mitochondrial chromosome. The solid line marks the p-value cutoff at 9.5*10⁻⁸ and the dashed line marks the p-value cutoff at 10⁻³.

FIG. 4 shows the effects of dropout and batch normalization on the 5,273-SNP DNN model. At the X-axis value of 100, the lines represent, from top to bottom, “DNN with dropout and batch normalization”, “DNN with dropout and without batch normalization”, “DNN without dropout and without batch normalization”, and “DNN without dropout and with batch normalization”.

FIG. 5 shows a comparison of machine learning approaches for PRS estimation. The performances of the models were represented as Receiver Operating Characteristic (ROC) curves. At the X-axis value of 0.4, the top solid line represents “DNN” and the bottom solid line represents “Decision Tree”. The Area under the ROC curve (AUC) and the accuracy from the test set are shown in the legend. The DNN model outperformed the other machine learning models in terms of AUC and accuracy.

FIG. 6 shows score histograms of DNN, BLUP, BayesA, and LDpred. The case and control populations are shown in the right-shifted and left-shifted histograms, respectively. The vertical line represents the score cutoff corresponding to the precision of 90% for each model. DNN had a much higher recall than the other algorithms at 90% precision.

FIG. 7 shows an example of a LINA model for structured data. The LINA model uses an input layer and multiple hidden layer to output the attention weights in the attention layer. The attention weights are then multiplied with the input features element-wise in the linearization layer and then with the coefficients in the output layer. The crossed neurons in the linearization layer represent element-wise multiplication of their two inputs. The incoming connections to the crossed neurons have a constant weight of 1.

FIG. 8 shows a first-order model-wise interpretation. The three bars of a feature represent the FP, DP, and IP scores, from left to right, of this feature in the LINA model.

FIG. 9 shows a second-order model-wise interpretation. The second-order modelwise importance scores (SP) are undirected between two features and are shown in a symmetric matrix as a heatmap. The importance scores for the feature self-interactions are set to zero in the diagonal of the matrix.

FIG. 10 shows a schematic of an MTL deep neural network for parallel prediction of multiple traits. This model was constructed based on the linearizing neural network architecture. The input layer (diamond box) contains all genetic variants in the whole genome. An attention vector is generated after 3 hidden layers (rectangular boxes) and then multiplied element-wise (round circle) with the input vector through a skip connection. The shared representation is used to predict each trait (y; in round circle). From end to end, a whole-phenome vector (diamond box) composed of many individual traits is predicted from this individual's whole-genome vector.

FIGS. 11A-11C show an estimation of PRS for malignant melanoma by STL and MTL. Density plots of malignment melanoma PRS estimated by (FIG. 11A) STL, (FIG. 11B) pan-cancer MTL, and (FIG. 11C) pan-disease MTL. Each panel contains two overlapping density plots: one for the control test cohort and one for the case test cohort. In FIG. 11A, the case curve reaches a higher value on the Y-axis than the Control curve. Iin FIGS. 11B and 11C, the Control curves reach a high value on the Y-axis than the Case curves. The separation between the control and case density plots is greater in the two MTL panels than in the STL panel.

FIGS. 12A-12B show PRS ROC AUC and PR AUC curves for malignant melanoma by STL and MTL. (FIG. 12A) Receiver operating characteristic (ROC) curves of STL, pan-cancer MTL, and pan-disease MTL for malignant melanoma PRS with the baseline (dotted line). At the X-axis value of 0.4, the top solid line represents “Pan-cancer MTL” and the bottom solid line represents “STL”. Both pan-cancer MTL and pan-disease MTL have larger ROC AUC than STL. (FIG. 12B) Precision-recall (PR) curves of STL, pancancer MTL, and pan-disease MTL for malignant melanoma PRS with the disease prevalence as the baseline (dotted line). At the X-axis value of 0.4, the top solid line represents “Pan-cancer MTL” and the bottom solid line represents “STL”. The two MTL models also have larger PR AUC than STL.

FIGS. 13A-13B show importance scores of real and decoy SNPs for malignant melanoma PRS estimation by pan-cancer MTL. (FIG. 13A) Manhattan plots of real SNPs (black and grey dots) and decoy SNPs (light dray dots) by their importance scores. (FIG. 13B) Density plots of the importance scores of real SNPs (black curve) and decoy SNPs (gray curve). An estimated FDR of 5% (3091 decoy SNP to 59,350 real SNPs) was reached at the importance score threshold of 0.52× 10³ (bottom dotted line). No decoy SNPs and 48 real SNPs have importance scores above the threshold of 3.25×10³ for an estimated 0.1% FDR (top dotted line).

FIG. 14 shows importance scores of real and decoy SNPs for malignant melanoma PRS estimation by pan-cancer MTL. The Venn diagrams show the overlap among the important SNPs found for uterine cancer, malignant melanoma, and colorectal cancer at 0.1% FDR (A) and 5% FDR (B). The sizes of the circles and their overlaps are drawn proportionally. The important sets of SNPs for the three cancers have a small overlap at 0.1% FDR and a large overlap at 5% FDR.

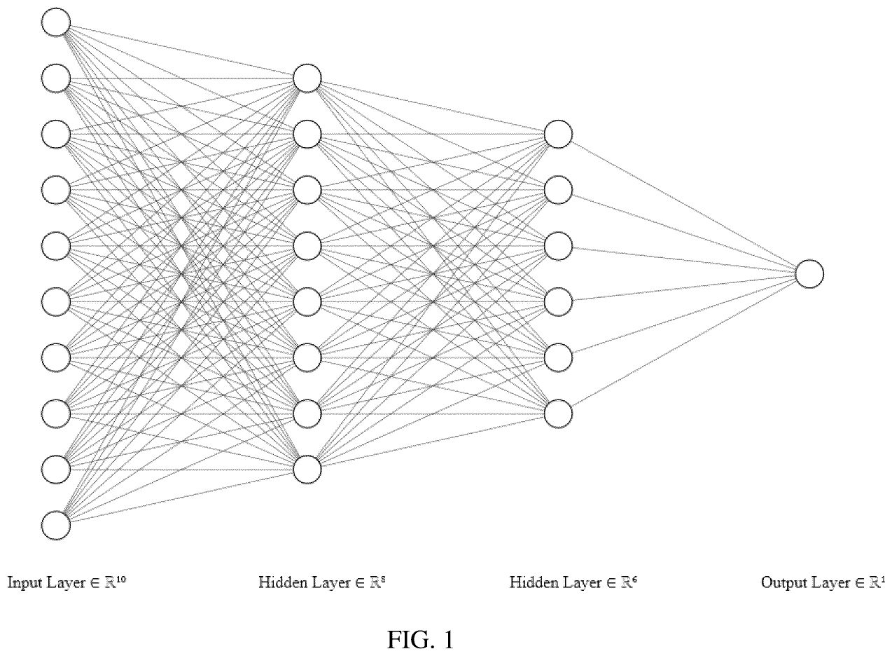

### DETAILED DESCRIPTION

The present disclosure is directed to methods of using multi-task learning to predict a subject's phenome-wide polygenic risk score (PRS) for developing a disease or condition. Various machine learning and statistical models for estimating breast cancer PRS were compared. A deep neural network (DNN) was found to be the most effective, outperforming other techniques such as Best Linear Unbiased Prediction (BLUP), BayesA, and LDpred. In the test cohort with 50% prevalence, the receiver operating characteristic curves area under the curves (ROC AUCs) were 67.4% for DNN, 64.2% for BLUP, 64.5% for BayesA, and 62.4% for LDpred. While BLUP, BayesA, and LDpred generated PRS that followed a normal distribution in the case population, the PRS generated by DNN followed a bimodal distribution. This allowed DNN to achieve a recall of 18.8% at 90% precision in the test cohort, which extrapolates to 65.4% recall at 20% precision in a general population. Interpretation of the DNN model identified significant variants that were previously overlooked by Genome-wide association studies (GWAS), highlighting their importance in predicting breast cancer risk.

A linearizing neural network architecture (LINA) that provided first-order and second-order interpretations on both the instance-wise and model-wise levels was developed, addressing the challenge of interpretability in neural networks. LINA outperformed other algorithms in providing accurate and versatile model interpretation, as demonstrated in synthetic datasets and real-world predictive genomics applications, by identifying salient features and feature interactions used for predictions.

Finally, overlapping genetic factors, such as pleiotropy or shared etiology was used to improve the accuracy of PRSs for multiple diseases simultaneously. Using an interpretable multi-task learning approach based on the LINA architecture, we found that the parallel estimation of PRS for 17 prevalent cancers using a pan-cancer MTL model was generally more accurate than independent estimations for individual cancers using comparable single-task learning models. Similar performance improvements were observed for 60 prevalent non-cancer diseases in a pan-disease MTL model. Interpretation of the MTL models revealed significant genetic correlations between important sets of single nucleotide polymorphisms, demonstrating that there is a well-connected network of diseases with a shared genetic basis.

In one embodiment, provided herein are methods for determining a PRS for a disease or condition for a subject. In some aspects, the methods comprise (a) obtaining a plurality of SNPs from the genome of the subject; (b) generating a data input from the plurality of SNPs; and (c) determining the polygenic risk score for the disease by applying to the data input a deep neural network trained by a Multi-task learning (MTL) model. In some aspects, the methods further comprise performing, or having performed, further screening for the disease or condition, or adjustments in the subject's medications, behavior, diet, or activities, if the PRS indicates that the subject is at risk for the disease. In some aspects, the disease is breast cancer, and wherein the method comprises performing, or having performed, yearly breast MRI and mammogram if the subject's PRS is greater than a predetermined threshold, such as for example, 20%.

Before further describing various embodiments of the apparatus, component parts, and methods of the present disclosure in more detail by way of exemplary description, examples, and results, it is to be understood that the embodiments of the present disclosure are not limited in application to the details of apparatus, component parts, and methods as set forth in the following description. The embodiments of the apparatus, component parts, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, component parts, and methods of the present disclosure have been described in terms of particular embodiments, it will be apparent to those of skill in the art that variations may be applied to the apparatus, component parts, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein. In particular, the entire contents of U.S. provisional application No. 63/241,645, filed Sep. 8, 2021, and U.S. Ser. No. 17/930,505, filed Sep. 8, 2022, are expressly incorporated herein by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms and phrases, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The phrase “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the apparatus, composition, or the methods or the variation that exists among the objects, or study subjects. As used herein the qualifiers “about” or “approximately” are intended to include not only the exact value, amount, degree, orientation, measuring error, manufacturing tolerances, stress exerted on various parts or components, observer error, wear and tear, and combinations thereof, for example.

The terms “about” or “approximately”, where used herein when referring to a measurable value such as an amount, percentage, temporal duration, and the like, is meant to encompass, for example, variations of +25% or +20% or +10%, or +5%, or +1%, or +0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. A range is intended to include any sub-range therein, although that sub-range may not be explicitly designated herein. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 2-125 therefore includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, and 125, as well as sub-ranges within the greater range, e.g., for 2-125, sub-ranges include but are not limited to 2-50, 5-50, 10-60, 5-45, 15-60, 10-40, 15-30, 2-85, 5-85, 20-75, 5-70, 10-70, 28-70, 14-56, 2-100, 5-100, 10-100, 5-90, 15-100, 10-75, 5-40, 2-105, 5-105, 100-95, 4-78, 15-65, 18-88, and 12-56. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

The terms “increase,” “increasing,” “enhancing,” or “enhancement” are defined as indicating a result that is greater in magnitude than a control number derived from analysis of a cohort, for example, the result can be a positive change of at least 5%, 10%, 20%, 30%, 40%, 50%, 80%, 100%, 200%, 300% or even more in comparison with the control number. Similarly, the terms “decrease,” “decreasing,” “lessening,” or “reduction” are defined as indicating a result that is lesser in magnitude than a control number, for example, the result can be a negative change of at least 5%, 10%, 20%, 30%, 40%, 50%, 80%, 100%, 200%, 300% or even more in comparison with the control number.

A polygenic risk score (PRS) is an estimate of the genetic propensity toward a phenotypic trait at the individual level. For example, if the phenotype under consideration is breast cancer, then the PRS should express the genetic risk (probability) of an individual getting breast cancer based on their particular combination of at-risk alleles (e.g., SNPs that are more prevalent in people who get breast cancer than those who don't). The application of this information could be, for example, to inform a woman of her PRS for breast cancer, wherein the woman is thereby able to take certain actions to reduce the likelihood of actually contracting breast cancer when her PRS for breast cancer indicates an increased risk.

In certain embodiments, the PRS cutoff (threshold) can be determined based on an absolute risk increase above a control number, e.g., an increase of about 1%, or about 2%, or about 3%, or about 4%, or about 5%, or about 6%, or about 7%, or about 8%, or about 9%, or about 10%, or about 15%, or about 20%, or about 25%, or about 30%, or about 35%, or about 40%, or about 45%, or about 50%, or about 55%, or about 60%, or about 65%, or about 70%, or about 75%, or about 80%, or about 85%, or about 90%, or about 95%, or about 100%, or about 150%, or about 200%, or about 250%, or about 300%, or greater. In one embodiment, the PRS cutoff is determined based on an absolute risk increase of 10% above the control number.

In some aspects, the disease risk is determined based on a PRS risk score cutoff (threshold) value. For instance, such a cutoff can include the highest about 1% in a PRS distribution, the highest about 2% in a PRS distribution, the highest about 3% in a PRS distribution, the highest about 4% in a PRS distribution, or the highest about 5% in a PRS distribution, the highest about 6% in a PRS distribution, the highest about 7% in a PRS distribution, the highest about 8% in a PRS distribution, the highest about 9% in a PRS distribution, the highest about 10% in a PRS distribution, the highest about 11% in a PRS distribution, the highest about 12% in a PRS distribution, the highest about 13% in a PRS distribution, the highest about 14% in a PRS distribution, the highest about 15% in a PRS distribution, the highest about 16% in a PRS distribution, the highest about 179% in a PRS distribution, the highest about 18% in a PRS distribution, the highest about 19% in a PRS distribution, the highest about 20% in a PRS distribution, the highest about 25% in a PRS distribution, the highest about 30% in a PRS distribution, the highest about 35% in a PRS distribution, the highest about 40% in a PRS distribution, the highest about 45% in a PRS distribution, the highest about 50% in a PRS distribution, the highest about 55% in a PRS distribution, the highest about 60% in a PRS distribution, the highest about 65% in a PRS distribution, the highest about 70% in a PRS distribution, the highest about 75% in a PRS distribution, or greater.

While the present method is discussed in some cases herein in the context of breast cancer, the methods used herein can be applied to a variety diseases and conditions, and in particular genetically complex diseases, such as, for example, other types of cancer, diabetes, neurological disorders, and neuromuscular disorders, and others as discussed below.

Deep learning generally refers to methods that map data through multiple levels of abstraction, where higher levels represent more abstract entities. The goal of deep learning is to provide a fully automatic system for learning complex functions that map inputs to outputs, without using hand crafted features or rules. One implementation of deep learning comes in the form of feedforward neural networks, where levels of abstraction are modeled by multiple non-linear hidden layers.

On average, SNPs can occur at approximately 1 in every 300 bases and as such there can be about 10 million SNPs in the human genome. In some cases, the deep neural network is trained with a labeled dataset comprising at least about 1,000, at least about 2,000, at least about 3,000, at least about 4,000, at least about 5,000, at least about 10,000, at least about 15,000, at least about 18,000, at least about 20,000, at least about 21,000, at least about 22,000, at least about 23,000, at least about 24,000, at least about 25,000, at least about 26,000, at least about 28,000, at least about 30,000, at least about 35,000, at least about 40,000, or at least about 50,000 SNPs.

In some cases, the neural network may be trained such that a desired accuracy of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The accuracy of PRS calling may be calculated as the percentage of patients with a known disease state that are correctly identified or classified as having or not have the disease.

In some cases, the neural network may be trained such that a desired sensitivity of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The sensitivity of PRS calling may be calculated as the percentage of patient's having a disease that are correctly identified or classified as having the disease.

In some cases, the neural network may be trained such that a desired specificity of PRS calling is achieved (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The specificity of PRS calling may be calculated as the percentage of healthy patients that are correctly identified or classified as not having a disease.

In some cases, the methods, systems, and devices of the present disclosure are applicable to diagnose, prognosticate, or monitor disease progression in a subject. For example, a subject can be a human patient, such as a cancer patient, a patient at risk for cancer, a patient suspected of having cancer, or a patient with a family or personal history of cancer. The sample from the subject can be used to analyze whether or not the subject carries SNPs that are implicated in certain diseases or conditions, e.g., cancer, Neurofibromatosis 1, McCune −Albright, incontinentia pigmenti, paroxysmal nocturnal hemoglobinuria, Proteus syndrome, or Duchenne Muscular Dystrophy. The sample from the subject can be used to determine whether or not the subject carries SNPs and can be used to diagnose, prognosticate, or monitor any cancer, e.g., any cancer disclosed herein. Examples of diseases and conditions that can be evaluated using the methods of the present disclosure include, but are not limited to, those listed below in Tables 13-17.

In certain aspects, the present disclosure provides a method for determining a PRS for a subject, and diagnosing, prognosticating, or monitoring the disease in the subject. In some cases, the method further comprises providing treatment recommendations or preventative monitoring recommendations for the disease, e.g., the cancer. In some cases, the cancer is selected from the group comprising: adrenal cancer, anal cancer, basal cell carcinoma, bile duct cancer, bladder cancer, cancer of the blood, bone cancer, a brain tumor, breast cancer, bronchus cancer, cancer of the cardiovascular system, cervical cancer, colon cancer, colorectal cancer, cancer of the digestive system, cancer of the endocrine system, endometrial cancer, esophageal cancer, eye cancer, gallbladder cancer, a gastrointestinal tumor, hepatocellular carcinoma, kidney cancer, hematopoietic malignancy, laryngeal cancer, leukemia, liver cancer, lung cancer, lymphoma, melanoma, mesothelioma, cancer of the muscular system, Myelodysplastic Syndrome (MDS), myeloma, nasal cavity cancer, nasopharyngeal cancer, cancer of the nervous system, cancer of the lymphatic system, oral cancer, oropharyngeal cancer, osteosarcoma, ovarian cancer, pancreatic cancer, penile cancer, pituitary tumors, prostate cancer, rectal cancer, renal pelvis cancer, cancer of the reproductive system, cancer of the respiratory system, sarcoma, salivary gland cancer, skeletal system cancer, skin cancer, small intestine cancer, stomach cancer, testicular cancer, throat cancer, thymus cancer, thyroid cancer, a tumor, cancer of the urinary system, uterine cancer, vaginal cancer, vulvar cancer, and any combination thereof.

In some cases, the determination of a PRS can provide valuable information for guiding the therapeutic intervention, e.g., for the cancer of the subject. For instance, SNPs can directly affect drug tolerance in many cancer types; therefore, understanding the underlying genetic variants can be useful for providing precision medical treatment of a cancer patient. In some cases, the methods, systems, and devices of the present disclosure can be used for application to drug development or developing a companion diagnostic. In some cases, the methods, systems, and devices of the present disclosure can also be used for predicting response to a therapy. In some cases, the methods, systems, and devices of the present disclosure can also be used for monitoring disease progression. In some cases, the methods, systems, and devices of the present disclosure can also be used for detecting relapse of a condition, e.g., cancer. A presence or absence of a known somatic variant or appearance of new somatic variant can be correlated with different stages of disease progression, e.g., different stages of cancers. As cancer progresses from early stage to late stage, an increased number or amount of new mutations can be detected by the methods, systems, or devices of the present disclosure.

Methods, systems, and devices of the present disclosure can be used to analyze biological samples from a subject. The subject can be any human being. The biological sample for PRS determination can be obtained from a tissue of interest, e.g., a pathological tissue, e.g., a tumor tissue. Alternatively, the biological sample can be a liquid biological sample containing cell-free nucleic acids, such as blood, plasma, serum, saliva, urine, amniotic fluid, pleural effusion, tears, seminal fluid, peritoneal fluid, and cerebrospinal fluid. Cell-free nucleic acids can comprise cell-free DNA or cell-free RNA. The cell-free nucleic acids used by methods and systems of the present disclosure can be nucleic acid molecules outside of cells in a biological sample. Cell-free DNA can occur naturally in the form of short fragments.

A subject applicable by the methods of the present disclosure can be of any age and can be an adult, infant or child. In some cases, the subject is within any age range (e.g., between 0 and 20 years old, between 20 and 40 years old, or between 40 and 90 years old, or even older). In some cases, the subjects as described herein can be a non-human animals such as dogs, cats, rats, mice, guinea pigs, chinchillas, horses, donkeys, goats, cattle, sheep, camelids, zoo animals, Old and New World monkeys, and non-human primates such as chimpanzees.

The use of the deep neural network can be performed with a total computation time (e.g., runtime) of no more than about 7 days, no more than about 6 days, no more than about 5 days, no more than about 4 days, no more than about 3 days, no more than about 48 hours, no more than about 36 hours, no more than about 24 hours, no more than about 22 hours, no more than about 20 hours, no more than about 18 hours, no more than about 16 hours, no more than about 14 hours, no more than about 12 hours, no more than about 10 hours, no more than about 9 hours, no more than about 8 hours, no more than about 7 hours, no more than about 6 hours, no more than about 5 hours, no more than about 4 hours, no more than about 3 hours, no more than about 2 hours, no more than about 60 minutes, no more than about 45 minutes, no more than about 30 minutes, no more than about 20 minutes, no more than about 15 minutes, no more than about 10 minutes, or no more than about 5 minutes.

In some cases, the methods and systems of the present disclosure may be performed using a single-core or multi-core machine, such as a dual-core, 3-core, 4-core, 5-core, 6-core, 7-core, 8-core, 9-core, 10-core, 12-core, 14-core, 16-core, 18-core, 20-core, 22-core, 24-core, 26-core, 28-core, 30-core, 32-core, 34-core, 36-core, 38-core, 40-core, 42-core, 44-core, 46-core, 48-core, 50-core, 52-core, 54-core, 56-core, 58-core, 60-core, 62-core, 64-core, 96-core, 128-core, 256-core, 512-core, or 1,024-core machine, or a multi-core machine having more than 1,024 cores. In some cases, the methods and systems of the present disclosure may be performed using a distributed network, such as a cloud computing network, which is configured to provide a similar functionality as a single-core or multi-core machine.

Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that can bear the software elements includes optical, electrical and electromagnetic waves, such as used across physical interfaces between local devices, through wired and optical landline networks and over various air-links. The physical elements that carry such waves, such as wired or wireless links, optical links or the like, also can be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as can be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media can be involved in carrying one or more sequences of one or more instructions to a processor for execution.

Any of the methods described herein can be totally or partially performed with a computer system including one or more processors, which can be configured to perform the operations disclosed herein. Thus, embodiments can be directed to computer systems configured to perform the operations of any of the methods described herein, with different components performing a respective operation or a respective group of operations. Although presented as numbered operations, the operations of the methods disclosed herein can be performed at a same time or in a different order. Additionally, portions of these operations can be used with portions of other operations from other methods. Also, all or portions of an operation can be optional. Additionally, any of the operations of any of the methods can be performed with modules, units, circuits, or other approaches for performing these operations.

Where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Where used herein, the pronoun “we” is intended to refer to all persons involved in a particular aspect of the work disclosed herein and as such may include non-inventor laboratory assistants and collaborators working under the supervision of the inventors.

The present disclosure will now be discussed in terms of several specific, non-limiting, examples and embodiments. The examples described below, which include particular embodiments, will serve to illustrate the practice of the present disclosure, it being understood that the particulars shown are by way of example and for purposes of illustrative discussion of particular embodiments and are presented in the cause of providing what is believed to be a useful and readily understood description of procedures as well as of the principles and conceptual aspects of the present disclosure.

### I.

False-positive mammography results, which typically lead to unnecessary follow-up diagnostic testing, have become increasingly common for women 40 to 49 years old (Nelson et al., 2009). Nevertheless, for women with a high risk for breast cancer (i.e. a lifetime risk of breast cancer higher than 20%), the American Cancer Society advises a yearly breast MRI and mammogram starting at 30 years of age (Oeffinger et al., 2015).

PRS assess the genetic risks of complex diseases based on the aggregate statistical correlation of a disease outcome with many genetic variations over the whole genome. Single-nucleotide polymorphisms (SNPs) are the most commonly used genetic variations. While genome-wide association studies (GWAS) report only SNPs with statistically significant associations to phenotypes (Dudbridge, 2013), PRS can be estimated using a greater number of SNPs with higher adjusted p-value thresholds to improve prediction accuracy. Previous research has developed a variety of PRS estimation models based on Best Linear Unbiased Prediction (BLUP), including gBLUP (Clark et al., 2013), rr-BLUP (Whittaker et al., 2000a), (Meuwissen et al., 2001), and other derivatives (Maier et al., 2015; Speed and Balding, 2014).

These linear mixed models consider genetic variations as fixed effects and use random effects to account for environmental factors and individual variability. Furthermore, linkage disequilibrium was utilized as a basis for the LDpred (Vilhj′almsson et al., 2015), (Khera et al., 2018), and PRS-CS (Ge et al., 2019) algorithms PRS estimation can also be defined as a supervised classification problem. The input features are genetic variations, and the output response is the disease outcome. Thus, machine learning techniques can be used to estimate PRS based on the classification scores achieved (Ho et al., 2019). A large-scale GWAS dataset may provide tens of thousands of individuals as training examples for model development and benchmarking. Wei et al (2019) (Wei et al., 2009) compared support vector machine and logistic regression to estimate PRS of Type-1 diabetes. The best Area Under the receiver operating characteristic Curve (AUC) was 84% in this study. More recently, neural networks have been used to estimate human height from the GWAS data, and the best R2 scores were in the range of 0.4 to 0.5 (Bellot et al., 2018). Amyotrophic lateral sclerosis was also investigated using Convolutional Neural Networks (CNN) with 4511 cases and 6127 controls (Yin et al., 2019) and the highest accuracy was 76.9%. Significant progress has been made in estimating PRS for breast cancer from a variety of populations.

In a recent study (Mavaddat et al., 2019), multiple large European women cohorts were combined to compare a series of PRS models. The most predictive model in this study used lasso regression with 3,820 SNPs and obtained an AUC of 65%. A PRS algorithm based on the sum of log odds ratios of important SNPs for breast cancer was used in the Singapore Chinese Health Study (Chan et al., 2018) with 46 SNPs and 56.6% AUC, the Shanghai Genome-Wide Association Studies (Wen et al., 2016) with 44 SNPs and 60.6% AUC, and a Taiwanese cohort (Hsich et al., 2017) with 6 SNPs and 59.8% AUC. A pruning and thresholding method using 5,218 SNPs reached an AUC of 69% for the UK Biobank dataset (Khera et al., 2018). In this study, deep neural network (DNN) was tested for breast cancer PRS estimation using a large cohort containing 26053 cases and 23058 controls. The performance of DNN was shown to be significantly higher than alternative machine learning algorithms and other statistical methods in this large cohort. Furthermore, DeepLift (Shrikumar et al., 2017) and LIME (Ribeiro et al., 2016) were used to identify salient SNPs used by DNN for prediction.

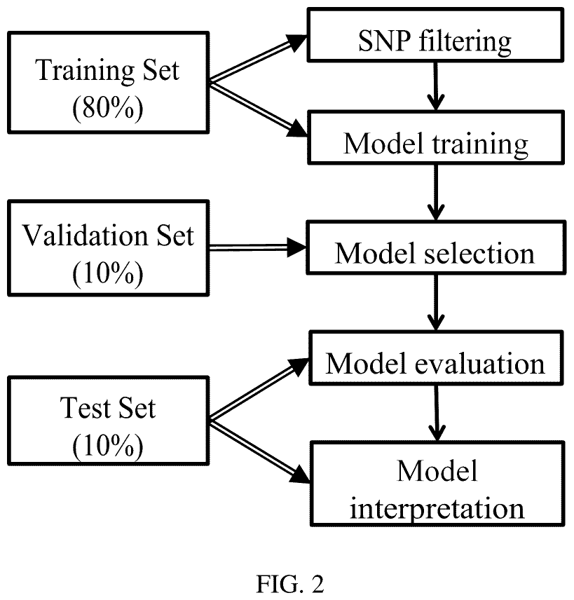

The present work used a breast cancer GWAS dataset generated by the Discovery, Biology, and Risk of Inherited Variants in Breast Cancer (DRIVE) project (Amos et al., 2017) and was obtained from the NIH dbGaP database under the accession number of phs001265.v1.pl. The DRIVE dataset was stored, processed, and used on the Schooner supercomputer at the University of Oklahoma in an isolated partition with restricted access. The partition consisted of 5 computational nodes, each with 40 CPU cores (Intel Xeon Cascade Lake) and 200 GB of RAM. The DRIVE dataset in the dbGap database was composed of 49,111 subjects genotyped for 528,620 SNPs using OncoArray (Amos et al., 2017). 55.4% of the subjects were from North America, 43.3% from Europe, and 1.3% from Africa. The disease outcome of the subjects was labeled as malignant tumor (48%), in situ tumor (5%), and no tumor (47%). In the present work, the subjects in the malignant tumor and in situ tumor categories were labeled as cases and the subjects in the no tumor category were labeled as controls, resulting in 26053 (53%) cases and 23058 (47%) controls. The subjects in the case and control classes were randomly assigned to a training set (80%), a validation set (10%), and a test set (10%) (FIG. 2). The association analysis was conducted on the training set using PLINK 2.0 (Chang et al., 2015). The p-value for each SNP was calculated using logistic regression.

Development of deep neural network models for PRS estimation

A variety of deep neural network (DNN) architectures (Bengio et al., 2009) were trained using Tensorflow 1.13. The Leaky Rectified Linear Unit (ReLU) activation function (Xu et al., 2015) was used on all hidden-layers neurons with the negative slope co-efficient set to 0.2. The output neuron used a sigmoid activation function. The training error was computed using the cross-entropy function:

$${\sum }_{i = 1}^{n} y * log ( p ) + ( 1 - y ) * log ( 1 - p ) \qquad ( 2.1 )$$

where p∈[0, 1] is the prediction probability from the model and y∈[10, 11] is the prediction target at 1 for case and 0 for control. DNNs were trained using minibatches with a batch size of 512. The Adam optimizer (Kingma and Ba, 2014b), an adaptive learning rate optimization algorithm, was used to update the weights in each mini-batch. The initial learning rate was set to 10⁻⁴, and the models were trained for up to 200 epochs with early stopping based on the validation AUC score. Dropout (Srivastava et al., 2014) was used to reduce overfitting. Batch normalization (BN) (Ioffe and Szegedy, 2015) was used to accelerate the training process, and the momentum for the moving average was set to 0.9 in BN.

Development of alternative machine learning models for PRS estimation

Logistic regression, decision tree, random forest, AdaBoost, gradient boosting, support vector machine (SVM), and Gaussian naive Bayes were implemented and tested using the scikit-learn machine learning library in Python. These models were trained using the same training set as the DNNs and, similarly, their hyperparameters were tuned using the same validation set (FIG. 2). These models are briefly described below.
(1) Decision Tree: The gini information gain with best split was used. The maximum depth was not set. The tree expanded until all leaves were pure or contained less than a minimum number of two examples per split,
(2) Random Forest: 3000 decision trees (as configured above) were used as base learners. Bootstrap samples were used to build each base learner. When searching for each tree's best split, the maximum number of considered features was set to be the square root of the number of features,
(3) AdaBoost: 2000 decision trees (as configured above) were used as base learners. The learning rate was set to 1. The algorithm used was SAMME.R (Hastie et al., 2009),
(4) Gradient Boosting: 400 decision trees (as configured above) were used as the base learners. Log-loss was used as the loss function. The learning rate was fixed to 0.1. The mean squared error with improvement score (Friedman, 2001) was used to measure the quality of a split,
(5) SVM: The kernel was a radial basis function with

$$γ = \frac{1}{n * Var} ,$$

where n is the number of SNPs and Var is the variance of the SNPs across individuals. The regularization parameter C was set to 1,
(6) Logistic Regression: L2 regularization with α=0.5 was used. L1 regularization was tested, but not used, because it did not improve the performance, and
(7) Gaussian Naive Bayes: The likelihood of the features was assumed to be Gaussian. The classes had uninformative priors.
   Development of statistical models for PRS estimation

The same training and validation sets were used to develop statistical models (FIG. 2). The BLUP and BayesA models were constructed using the bWGR R package. The LDpred model was constructed using the algorithm as described (Vilhj′almsson et al., 2015): (1) BLUP: The linear mixed model was y=μ+Xb+e, where y were the response variables, μ were the intercepts, X were the input features, b were the regression coefficients, and e were the residual coefficients, (2) BayesA: The priors were assigned from a mixture of normal distributions, (3) LDpred: The p-values were generated by our association analysis described above. The validation set was provided as reference for LDpred data coordination. The radius of the Gibbs sampler was set to be the number of SNPs divided by 3000 as recommended by the LDpred user manual.

The score distributions of DNN, BayesA, BLUP, and LDpred were analyzed with the Shapiro test for normality and the Bayesian Gaussian mixture (BGM) expectation maximization algorithm. The BGM algorithm decomposed a mixture of two Gaussian distributions with weight priors at 50.

DNN model interpretation protocol

LIME and DeepLift were used to interpret the DNN predictions for subjects in the test set with DNN output scores higher than 0.67, which corresponded to a precision of 90%. For LIME, the submodular pick algorithm was used, the kernel size was set to 40, and the number of explainable features was set to 41. For DeepLift, the importance of each SNP was computed as the average across all individuals, and the reference activation value for a neuron was determined by the average value of all activations triggered across all subjects.

### Results

Development of a machine learning model for breast cancer PRS estimation

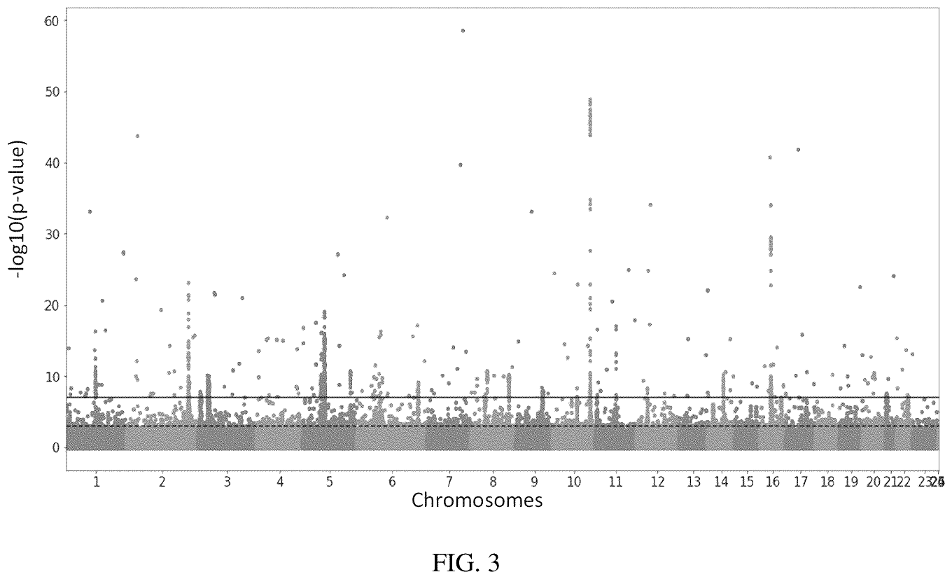

The breast cancer GWAS dataset containing 26053 cases and 23058 controls was generated by the Discovery, Biology, and Risk of Inherited Variants in Breast Cancer (DRIVE) project (Amos et al., 2017). The DRIVE data is available from the NIH dbGaP database under the accession number of phs001265.v1.pl. As noted above, the cases and controls were randomly split into a training set, a validation set, and a test set (FIG. 2). The training set was used to estimate the p-values of SNPs using association analysis and train machine learning and statistical models. The hyperparameters of the machine learning and statistical models were optimized using the validation set. The test set was used for the final performance evaluation and model interpretation. The statistical significance of the disease association with 528,620 SNPs was assessed with Plink using only the training set. The obtained p-values for all tested SNPs are shown in FIG. 3 as a Manhattan plot. Table 2 shows performance results of the DNN models trained using five SNP sets filtered with increasing p-value cutoffs. The models were compared by their training costs and performances in the training and validation sets.

TABLE 2
Performance DNN models trained using five SNP sets

| | | Computational | | | | | |
|---|---|---|---|---|---|---|---|
| | | Cost of Training | | | | | |
| | | Convergence | Peak | | | | |
| p-value | | time | Memory | AUC | | Accuracy | |
| cutoff | SNPs | (minutes) | (GB) | Training | Validation | Training | Validation |
| None | 528,620 | 1308 | 66.6 | 100.0% | 65.9% | 100.0% | 60.1% |
| 10-2 | 13,890 | 51 | 3.2 | 93.4% | 66.5% | 85.1% | 61.4% |
| 10-3 | 5,273 | 23 | 2.2 | 80.5% | 67.1% | 73.4% | 62.0% |
| 10-4 | 3,041 | 16 | 2 | 75.9% | 66.4% | 67.6% | 61.1% |
| 10-5 | 2,099 | 9 | 1.4 | 72.2% | 65.7% | 63.2% | 60.8% |

To obtain unbiased benchmarking results on the test set, it was critical not to use the test set in the association analysis (FIG. 2) and not to use association p values from previous GWAS studies that included subjects in the test set, as well-described in the Section 7.10.2 of (Hastie et al., 2009). There were 1,061 SNPs with a p-value less than the critical value of 9.5*10⁻⁸, which was set using the Bonferroni correction (9.5×10₋₈=0.05/528,620). Filtering with a Bonferroni-corrected critical value may remove many informative SNPs that have small effects on the phenotype, epistatic interactions with other SNPs, or non-linear association with the phenotype (De et al., 2014). Relaxed filtering with higher p-value cutoffs was tested to find the optimal feature set for DNN (Table 2). The DNN models used to generate the results in Table 2 had a deep feedforward architecture consisting of an input layer of variable sizes, followed by 3 successive hidden layers containing 1000, 250, and 50 neurons and an output layer with a single neuron. As the p-value cutoff increased, a greater number of SNPs were incorporated as input features, and training consumed a larger amount of computational resources in terms of computing time and peak memory usage. A feature set containing 5,273 SNPs above the p-value cutoff of 10⁻³ provided the best prediction performance measured by the AUC and accuracy on the validation set. In comparison with smaller feature sets from more stringent p-value filtering, the 5,273-SNP feature set may have included many informative SNPs providing additional signals to be captured by DNN for prediction. On the other hand, more relaxed filtering with p-value cutoffs greater than 10⁻³ led to significant overfitting as indicated by an increasing prediction performance in the training set and a decreasing performance in the validation set (Table 2).

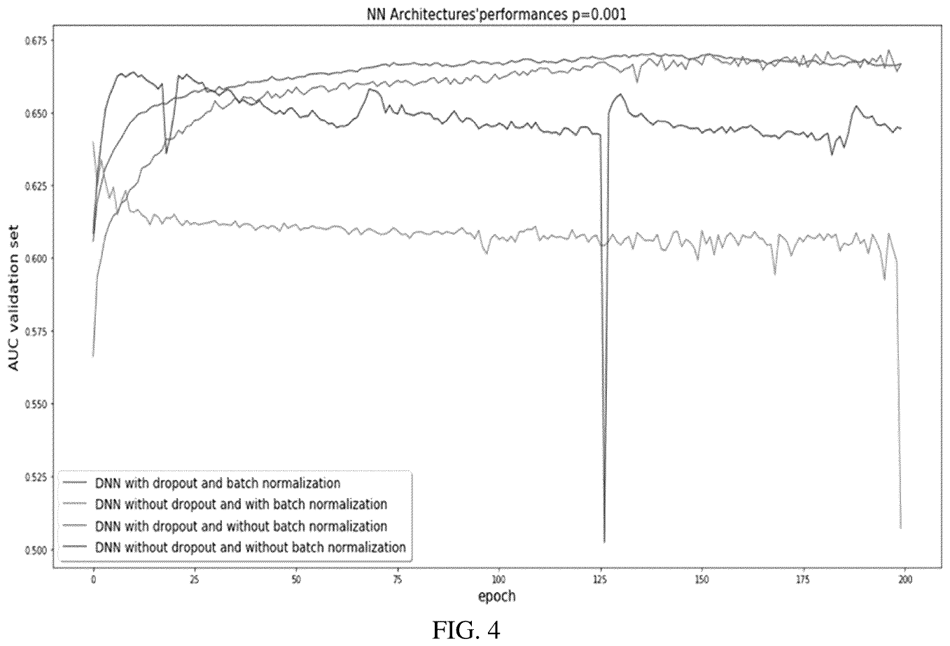

The largest DNN model, consisting of all 528,620 SNPs, decreased the validation AUC score by only 1.2% and the validation accuracy by 1.9% from the highest achieved values. This large DNN model used an 80% dropout rate to obtain strong regularization, while all the other DNN models utilized a 50% dropout rate. This suggested that DNN was able to perform feature selection without using p-values, although the limited training data and the large neural network size resulted in complete overfitting. The effects of dropout and batch normalization were tested using the 5,273-SNP DNN model (FIG. 4). Without dropout, the DNN model using only batch normalization had a 3.0% drop in AUC and a 4.0% drop in accuracy and its training converged in only two epochs. Without batch normalization, the DNN model had 0.1% higher AUC and 0.3% lower accuracy but its training required a 73% increase in the number of epochs to reach convergence. As an alternative to filtering, autoencoding was tested to reduce a large number of SNPs to a small set of features for PRS estimation (Fergus et al., 2018; Cudic et al., 2018). An autoencoder was trained to encode 5273 SNPs into 2000 features with a mean square error (MSE) of 0.053 and a root mean square error (RMSE) of 0.23. The encodings were used to train a DNN model with the same architecture as the ones shown in FIG. 2.2B except for the number of input neurons. The autoencoder-DNN model had a similar number of input neurons for DNN as the 2099-SNP DNN model, but had a 1.3% higher validation AUC and a 0.2% higher validation accuracy (Tables 2 and 3). This increased validation AUC and accuracy suggested that the autoencoding provided improved dimensionality reduction compared to the SNP filtering based on p-values. In comparison with the 5,273-SNP model, auto-encoding sped up the convergence process, but led to a 0.3% reduction in validation AUC score and a 1.6% reduction in validation accuracy.

The deep feedforward architecture benchmarked in Table 2 was compared with a number of alternative neural network architectures using the 5,273-SNP feature set (Table 3). A shallow neural network with only one hidden layer resulted in a 0.9% lower AUC and 1.1% lower accuracy in the validation set compared to the DNN. This suggested that additional hidden layers in DNN were useful in representing complex interactions among SNPs. The additional hidden layers also supported additional feature selection and transformation in the model. One-dimensional convolutional neural network (1D CNN) was previously used to estimate the PRS for bone heel mineral density, body mass index, systolic blood pressure and waist-hip ratio (Bellot et al., 2018) and was also tested here for breast cancer prediction with the DRIVE dataset.

TABLE 3
Effects of dropout and batch normalization on the 5,273-SNP DNN model.

| | | Validation | Validation | Convergence |
|---|---|---|---|---|
| Model | Architecture | AUC | Accuracy | (# Epochs) |
| DNN | 3 hidden layers with 1000, 250, and 50 | 67.1% | 62.0% | 110 |
| | neurons. Dropout and batch normalization | | | |
| | (BN) enabled | | | |
| Shallow NN (SNN) | 1 hidden layer with 50 neurons. With | 66.2% | 60.9% | 20 |
| | dropout but no BN | | | |
| 1D Convolutional NN | 2 convolution layers with max pooling | 63.9% | 59.9% | 155 |
| (1D CNN) | followed by 3 hidden layers with 1000, | | | |
| | 250, and 50 neurons. Dropout and BN | | | |
| | enabled | | | |
| Autoencoder-DNN | autoencoding with no hidden layer | 67.0% | 61.0% | 31 |
| | followed by DNN with dropout and BN | | | |
| | enabled | | | |

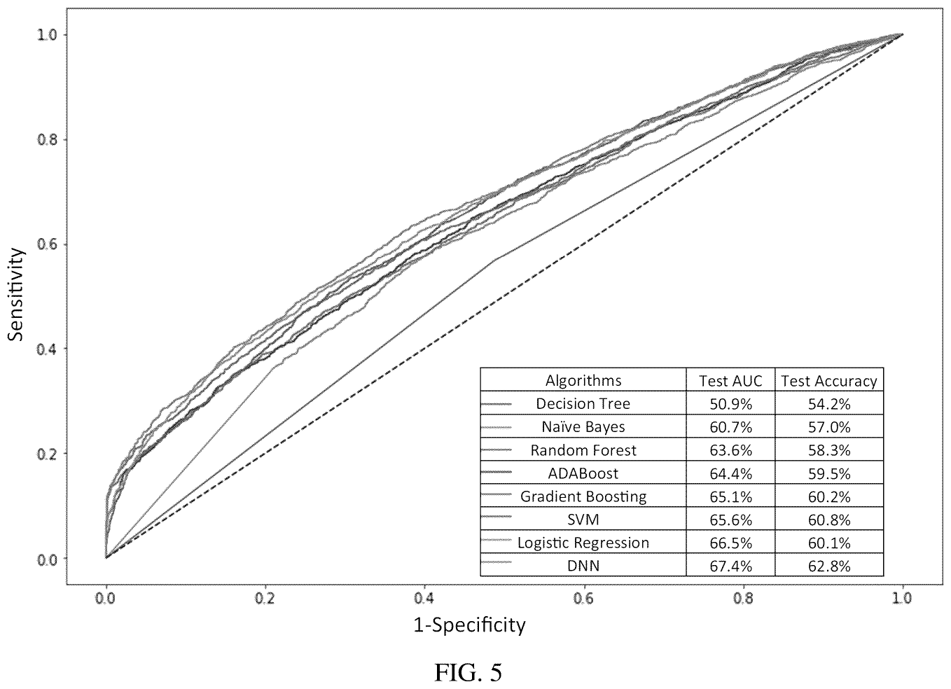

The validation AUC and accuracy of 1D CNN were lower than DNN by 3.2% and 2.0%, respectively. Two-dimensional CNN was particularly popular for image analysis, because the receptive field of the convolutional layer can capture space-invariant information with shared parameters. However, the SNPs distributed across a genome may not have significant space-invariant patterns to be captured by the convolutional layer, which may explain the poor performance of CNN. The 5,273-SNP feature set was used to test alternative machine learning approaches, including logistic regression, decision tree, naive Bayes, random forest, ADAboost, gradient boosting, and SVM, for PRS estimation (FIG. 5). These models were trained, turned, and benchmarked using the same training, validation, and test sets, respectively, as the DNN models (FIG. 2). Although the decision tree had a test AUC of only 50.9%, ensemble algorithms that used decision trees as the base learner, including random forest, ADABoost, and gradient boosting, reached test AUCs of 63.6%, 64.4%, and 65.1%, respectively. This showed the advantage of ensemble learning. SVM reached a test AUC of 65.6%. Na“ive Bayes and logistic regression were both linear models with the assumption of independent features. Logistic regression performed substantially better than naïve Bayes, ensemble techniques and SVM, based on the AUC scores. Out of all the machine learning models, the DNN model still achieved the highest test AUC at 67.4% and the highest test accuracy at 62.8%.

Comparison of the DNN model with statistical models for breast cancer PRS estimation

The performance of DNN was compared with three representative statistical models, including BLUP, BayesA, and LDpred (Table 4). Because the relative performance of these methods may be dependent on the number of training examples available, the original training set containing 39,289 subjects was down-sampled to create three smaller training sets containing 10000, 20000, 30000 subjects. As the 5,273-SNP feature set generated with a p-value cutoff of 10⁻³ may not be the most appropriate for the statistical methods, a 13,890-SNP feature set (p-value cutoff=10⁻²) and a 2,099-SNP feature set (p-value cutoff=10⁻⁵) were tested for all methods. Although LDpred also required training data, its prediction relied primarily on the provided p-values, which were generated for all methods using all 39,289 subjects in the training set. Thus, the down-sampling of the training set did not reduce the performance of LDpred. LDpred reached its highest AUC score at 62.4% using the p-value cutoff of 10⁻³. A previous study (Ge et al., 2019) [12] that applied LDpred to breast cancer prediction using the UK Biobank dataset similarly obtained an AUC score of 62.4% at the pvalue cutoff of 10⁻³. This showed consistent performance of LDpred in the two studies using different datasets.

When DNN, BLUP, and BayesA used the full training set, they obtained higher AUCs than LDpred at their optimum p-value cutoffs. DNN, BLUP, and BayesA all gained performance with the increase in the training set sizes (Table 4). The performance gain was more substantial for DNN than BLUP and BayesA. The increase from 10,000 subjects to 39,258 subjects in the training set resulted in a 1.9% boost to DNN's best AUC, a 0.7% boost to BLUP, and a 0.8% boost to BayesA. This indicated the different variance-bias trade-offs made by DNN, BLUP, and BayesA. The high variance of DNN required more training data, but could capture more extensive interactions among SNPs and non-linear relationships between the SNPs and the phenotype. The high bias of BLUP and BayesA had lower risk for overfitting using smaller training sets, but their models only considered linear relationships. The higher AUCs of DNN across all training set sizes indicated that DNN had a better variancebias balance for breast cancer PRS estimation.

For all four training set sizes, BLUP and BayesA achieved higher AUCs using more stringent p-value filtering. When using the full training set, reducing the p-value cutoffs from 10-2 to 10⁻⁵ increased the AUCs of BLUP from 61.0% to 64.2% and the AUCs of BayesA from 61.1% to 64.5%. This suggested that BLUP and BayesA preferred a reduced number of SNPs that were found by logistic regression to be significantly associated with the phenotype. On the other hand, DNN produced lower AUCs using the p-value cutoff of 10⁻⁵ than the other two higher cutoffs. This suggested that DNN can perform better feature selection in comparison to SNP filtering based on p-values from logistic regression.

TABLE 4
AUC test scores of DNN, BLUP, BayesA, and LDpred model at
different p-value cutoffs (PC) and training set sizes (TS)

| | Algorithms | | | | | | | | | | | |
|---|---|---|---|---|---|---|---|---|---|---|---|---|
| | DNN | | | BLUP | | | BayesA | | | LDpred | | |
| | PC* | | | | | | | | | | | |
| TS** | 10⁻⁵ | 10⁻³ | 10⁻² | 10⁻⁵ | 10⁻³ | 10⁻² | 10⁻⁵ | 10⁻³ | 10⁻² | 10⁻⁵ | 10⁻³ | 10⁻² |
| 10,000 | 64.8% | 65.5% | 65.1% | 63.5% | 62.5% | 60.6% | 63.7% | 62.0% | 59.9% | 60.8% | 62.4% | 61.6% |
| 20,000 | 65.6% | 66.6% | 66.4% | 62.9% | 63.0% | 60.6% | 62.7% | 63.0% | 60.4% | 60.8% | 62.4% | 61.6% |
| 30,000 | 66.0% | 66.9% | 66.6% | 64.2% | 63.1% | 60.7% | 64.3% | 63.1% | 60.7% | 60.7% | 62.4% | 61.6% |
| 39,289 | 66.2% | 67.4% | 67.3% | 64.2% | 63.3% | 61.0% | 64.5% | 63.4% | 61.1% | 60.7% | 62.4% | 61.6% |

*p-value cutoff
**training set size

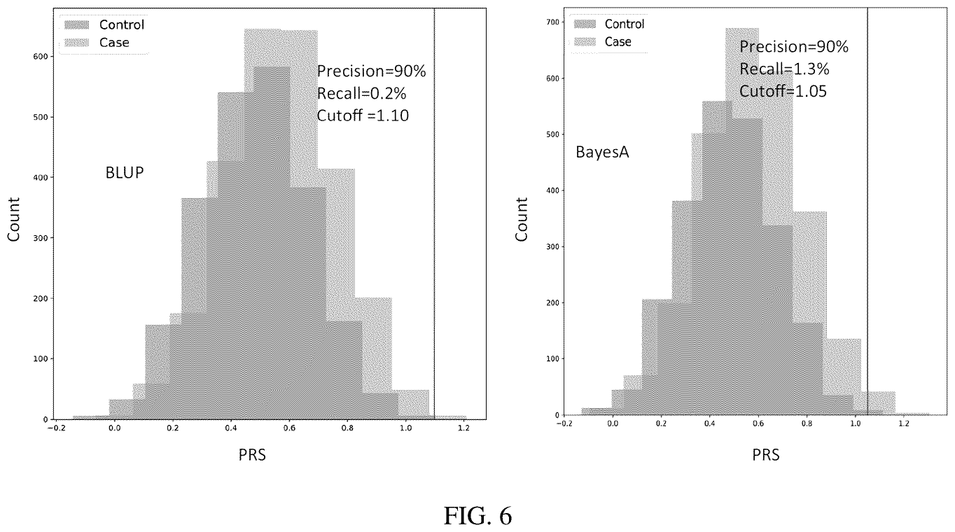

The four algorithms were compared using the score histograms of the case population and the control population from the test set in FIG. 6. The score distributions of BLUP, BayesA and LDpred all followed normal distributions. The four algorithms were compared using the score histograms of the case population and the control population from the test set in FIG. 6. The score distributions of BLUP, BayesA and LDpred all followed normal distributions. The p-values from the Shapiro normality test of the case and control distributions were 0.46 and 0.43 for BayesA, 0.50 and 0.95 for BLUP, and 0.17 and 0,24 for LDpred, respectively. The case and control distributions were

$${N}_{case} ( μ = 0.577 , σ = 0.2 ) and \\ {N}_{control} ( μ = 0.479 , σ = 0.19 ) from BayseA , \\ {N}_{cases} ( μ = 0.572 , σ = 0.19 ) and \\ {N}_{control} ( μ = 0.483 , σ = 0.18 ) from BLUP , and \\ {N}_{case} ( μ = - 33.52 , σ = 5.4 ) and \\ {N}_{control} ( μ = - 35.86 , σ = 4.75 ) from LDpred .$$

The means of the case distributions were all significantly higher than the control distributions for BayesA (p-value<10⁻¹⁶), BLUP (p-value<10⁻¹⁶), and LDpred (p-value<10⁻¹⁶) and their case and control distributions had similar standard deviations. The score histograms of DNN did not follow normal distributions based on the Shapiro normality test with a pvalue of 4.1×10⁻³⁴ for the case distribution and a p-value of 2.5×10⁻⁹ for the control distribution. The case distribution had the appearance of a bi-modal distribution. The Bayesian Gaussian mixture expectation maximization algorithm decomposed the case distribution to two normal distributions: N_{case}1 (μ=0.519,σ=0.096) with an 86.5% weight and N_{case}? (μ=0.876,σ=0.065) with a 13.5% weight.

The control distribution was resolved into two normal distributions with similar means and distinct standard deviations: N_{control} (μ=0.471,σ=0.1) with an 85.0% weight and N_{control} (μ=0.507,0=0.03) with a 15.0% weight. The N_{case1} distribution had a similar mean as the N_{control1} and N_{control2} distributions. This suggested that the N_{case1} distribution may represent a normal-genetic-risk case sub-population, in which the subjects may have a normal level of genetic risk for breast cancer and the oncogenesis likely involved a significant environmental component. The mean of the N_{case2} distribution was higher than the means of both the N_{case1} and N_{control1} distributions by more than 4 standard deviations (p-value<10⁻¹⁶). We hypothesized that the N_{case2} distribution represented a high-genetic-risk case sub-population for breast cancer, in which the subjects may have inherited many genetic variations associated with breast cancer.

Three GWAS were performed between the high-genetic risk case subpopulation with DNN PRS<0.67, the normal genetic-risk case subpopulation with DNN PRS<0.67, and the control population. The GWAS analysis of the high-genetic-risk case subpopulation versus the control population identified 182 significant SNPs at the Bonferroni level of statistical significance. The GWAS analysis of the high-genetic-risk case subpopulation versus the normal-genetic-risk case subpopulation identified 216 significant SNPs. The two sets of significant SNPs found by these two GWAS analyses were very similar, sharing 149 significant SNPs in their intersection. Genes associated with these 149 SNPs were investigated with pathway enrichment analysis (Fisher's Exact Test; P<0.05) using SNPnexus (Dayem Ullah et al., 2018) (see Supplementary Table 4 in Badré et al.,2021). Many of the significant pathways were involved in DNA repair (O'Connor, 2015) signal transduction (Kolch et al., 2015), and suppression of apoptosis (Fernald and Kurokawa, 2013). Interestingly, the GWAS analysis of the normal genetic-risk case subpopulation and the control population identified no significant SNP. This supported our classification of the cases into the normal-genetic-risk subjects, and Deep neural network improves the estimation of polygenic risk scores for breast cancer 365 the high-genetic-risk subjects based on their PRS scores from the DNN model.

In comparison with AUCs, it may be more relevant for practical applications of PRS to compare the recalls of different algorithms at a given precision that warrants clinical recommendations. At 90% precision, the recalls were 18.8% for DNN, 0.2% for BLUP, 1.3% for BayesA, and 1.3% for LDpred in the test set of the DRIVE cohort with a 50% prevalence. This indicated that DNN can make a positive prediction for 18.8% of the subjects in the DRIVE cohort and these positive subjects would have an average chance of 90% to eventually develop breast cancer. However, BLUP, BayesA and LDpred can only make a similarly confident prediction for less than 2% of the subjects. American Cancer Society advises yearly breast MRI and mammogram starting at the age of 30 years for women with a lifetime risk of breast cancer greater than 20%, which meant a 20% precision for PRS. By extrapolating the performance in the DRIVE cohort, the DNN model should be able to achieve a recall of 65.4% at a precision of 20% in the general population with a 12% prevalence rate of breast cancer.

Interpretation of the DNN model

While the DNN model used 5,273 SNPs as input, we hypothesized that only a small set of these SNPs were particularly informative for identifying the subjects with high genetic risks for breast cancer. LIME and DeepLift were used to find the top-100 salient SNPs used by the DNN model to identify the subjects with classification scores higher than the cutoff at 90% precision. 23 SNPs were ranked by both algorithms to be among their top-100 salient SNPs The small overlap between their results can be attributed to their different interpretation approaches. LIME considered the DNN model as a black box and perturbed the input to estimate the importance of each variable; whereas, DeepLift analyzed the gradient information of the DNN model. 30% of LIME's salient SNPs and 49% of DeepLift's salient SNPs had p-values less than the Bonferroni significance threshold of 9.5×10⁻⁸. This could be attributed to the non-linear relationship between the salient SNPs and the disease outcome, which cannot be captured by association analysis using logistic regression.

Michailidou et al., (2017) summarized a total of 172 SNPs associated with breast cancer. Out of these SNPs, 59 were not included on OncoArray, 63 had an association p value less than 103 and were not included in the 5273-SNP feature set for DNN, 34 were not ranked among the top-1000 SNPs by either DeepLIFT or LIME, and 16 were ranked among the top-1000 SNPs by DeepLIFT, LIME, or both (see Supplementary Table 5 in Badré et al., 2021). This indicates that many SNPs with significant association may be missed by the interpretation of DNN models.

The 23 salient SNPs identified by both DeepLift and LIME in their top-100 list are shown in Table 5. Eight of these SNPs had p-values higher than the Bonferroni level of significance and were missed by the association analysis using Plink. The potential oncogenesis mechanisms for some of the eight SNPs have been investigated in previous studies. The SNP, rs139337779 at 12q24.22, is located within the gene, Nitric oxide synthase 1 (NOS1). (Li et al., 2019) showed that the overexpression of NOS1 can upregulate the expression of ATP-binding cassette, subfamily G, member 2 (ABCG2), which is a breast cancer resistant protein (Mao and Unadkat, 2015), and NOS1-indeuced chemo-resistance was partly mediated by the upregulation of ABCG2 expression. (Lee et al., 2009) reported that NOSI is associated with the breast cancer risk in a Korean cohort. The SNP, chr13 113796587 A G at 13q34, is located in the F10 gene, which is the coagulation factor (Tinholt et al., 2014) showed that the increased coagulation activity and genetic polymorphisms in the F10 gene are associated with breast cancer. The BNC2 gene containing the SNP, chr9 16917672 G T at 9p22.2, is a putative tumor suppressor gene in high-grade serious ovarian carcinoma (Cesaratto et al., 2016). The SNP, chr2 171708059 C T at 2q31.1, is within the GADI gene and the expression level of GADI is a significant prognostic factor in lung adenocarcinoma (Tsuboi et al., 2019). Thus, the interpretation of DNN models may identify novel SNPs with nonlinear association with the breast cancer (Purcell Shaun et al., 2009; Scott et al., 2017; LeBlanc and Kooperberg, 2010; Angermueller et al., 2016; Schmidhuber, 2015).

TABLE 5
Top salient SNPs identified by both LIME and DeepLift from the DNN model

| | | LIME | DeepLift | | | |
|---|---|---|---|---|---|---|
| SNP | locus | (10⁻⁴) | (10⁻²) | p-value | MAF* | Genes of interest** |
| corect_rs139337779 | 12q24.22 | 4.5 | −3.3 | 6.5E−04 | 11% | NOS1 |
| chr13_113796587_A_G | 13q34 | 4.3 | −3.8 | 2.8E−04 | 3% | F10 |
| chr9_16917672_G_T | 9p22.2 | 4.5 | −2.5 | 7.6E−05 | 4% | BNC2/RP11- |
| | | | | | | 132E11.2 |
| chr8_89514784_A_G | 8q21.3 | 27.0 | 3.7 | 2.5E−05 | 56% | RP11-586K2.1 |
| chr17_4961271_G_T | 17p13.2 | 4.2 | −2.2 | 6.2E−06 | 4% | SLC52A1/RP11- |
| | | | | | | 46I8.1 |
| rs11642757 | 16q23.2 | 5.3 | −2.9 | 2.0E−06 | 6% | RP11-345M22.1 |
| rs4040605 | 1p36.33 | 4.4 | 2.4 | 9.6E−07 | 37% | RP11-54O7.3/ |
| | | | | | | SAMD11 |
| chrS_180405432_G_T | 5q35.3 | 4.1 | −3.4 | 2.3E−07 | 3% | CTD-2593A12.3/ |
| | | | | | | CTD-2593A12.4 |
| Chr6:30954121:G:T | 6p21.33 | 6.8 | 4.9 | 1.0E−08 | 42% | MUC21 |
| chr14_101121371_G_T | 14q32.2 | 5.8 | 3.9 | 1.0E−10 | 33% | CTD2644I21.1/ |
| | | | | | | LINC00523 |
| rs12542492 | 8q21.11 | 40.0 | 2.8 | 6.3E−11 | 34% | RP11-434I12.2 |
| corect_rs116995945 | 17q22 | 3.6 | −4.5 | 2.5E−11 | 5% | SCPEP1/RNF126R1 |
| chr14_76886176_C_T | 14q24.3 | 3.5 | 2.3 | 2.3E−11 | 41% | ESRR8 |
| chr2_171708059_C_T | 2q31.1 | 4.1 | −6.7 | 1.9E−11 | 7% | GAD1 |
| chr7_102368966_A_G | 7q22.1 | 4.1 | −2.6 | 6.8E−12 | 4% | RASA4DP/FAM185A |
| chr8_130380476_C_T | 8q24.21 | 4.3 | 2.5 | 4.7E−12 | 22% | CCDC26 |
| corect_rs181578054 | 22q13.33 | 4.1 | 3.0 | 7.1E−14 | 40% | ARSA/Y_RNA |
| rs3858522 | 11p15.5 | 7.7 | 3.3 | 2.2E−17 | 52% | H19/IGF2 |
| chr3_46742523_A_C | 3p21.31 | 5.2 | 4.9 | 1.8E−22 | 35% | ALS2CL/TMIE |
| chr13_113284191_C_T | 13q34 | 4.0 | −4.0 | 7.8E−23 | 5% | TU5GCP3/C13orf35 |
| chr1_97768640_A_G | 1p21.3 | 6.0 | −6.8 | 6.6E−34 | 9% | DPYD |
| chr7_118831547_C_T | 7q31.31 | 4.0 | −3.5 | 1.9E−40 | 6% | RP11-500M10.1/ |
| | | | | | | AC091320.2 |
| chr16_52328666_C_T | 16q12.1 | 23.0 | 5.2 | 1.5E−41 | 21% | RP11-142G1.2/TOX3 |

*Minor Allele Frequency
**<300 kb form target SNPs

### II.

### A Linearizing Neural Network Architecture (LINA) for Accurate First-Order and Second-Order Interpretations

An interpretable machine learning algorithm should have a high representational capacity to provide strong predictive performance, and its learned representations should be amenable to model interpretation and understandable to humans. The two desiderata are generally difficult to balance. Linear models and decision trees generate simple representations for model interpretation but have low representational capacities for only simple prediction tasks. Neural networks and support vector machines have high representational capacities to handle complex prediction tasks, but their learned representations are often considered to be “black boxes” for model interpretation (Bermeitinger. et al., 2019). Predictive genomics is an exemplary application that requires both a strong predictive performance and high interpretability. In this application, the genotype information for a large number of SNPs in a subject's genome is used to predict the phenotype of this subject. While neural networks have been shown to provide better predictive performance than statistical models (Badré et al., 2021; Fergus et al., 2018), statistical models are still the dominant methods for predictive genomics, because geneticists and genetic counselors can understand which SNPs are used and how they are used as the basis for certain phenotype predictions. Neural network models have also been used in many other important bioinformatics applications (Ho Thanh Lam et al., 2020; Do and Le, 2020; Baltres et al., 2020) that can benefit from model interpretation.

To make neural networks more useful for predictive genomics and other applications, we developed a new neural network architecture, referred to as linearizing neural network architecture (LINA), to provide both first-order and second-order interpretations and both instance-wise and model-wise interpretations. Model interpretation reveals the input-to-output relationships that a machine learning model has learned from the training data to make predictions (Molnar, 2020). The first-order model interpretation aims to identify individual features that are important for a model to make predictions. For predictive genomics, this can reveal which individual SNPs are important for phenotype prediction. The second-order model interpretation aims to identify important interactions among features that have a large impact on model prediction. The second-order interpretation may reveal the XOR interaction between the two features that jointly determine the output. For predictive genomics, this may uncover epistatic interactions between pairs of SNPs (Cordell, 2002; Phillips, 2008). A general strategy for the first-order interpretation of neural networks, first introduced by Saliency (Simonyan et al., 2014), is based on the gradient of the output with respect to (w.r.t.) the input feature vector. A feature with a larger partial derivative of the output is considered more important. The gradient of a neural network model w.r.t. the input feature vector of a specific instance can be computed using backpropagation, which generates an instance-wise first-order interpretation. The Grad*Input algorithm (Shrikumar et al., 2017) multiplies the obtained gradient element-wise with the input feature vector to generate better scaled importance scores. As an alternative to using the gradient information, the Deep Learning Important FeaTures (DeepLIFT) algorithm explains the predictions of a neural network by backpropagating the activations of the neurons to the input features (Shrikumar et al., 2017). The feature importance scores are calculated by comparing the activations of the neurons with their references, which allows the importance information to pass through a zero gradient during backpropagation.

The Class Model Visualization (CMV) algorithm (Simonyan et al., 2014) computes the visual importance of pixels in convolution neural network (CNN). It performs backpropagation on an initially dark image to find the pixels that maximize the classification score of a given class. While the algorithms described above were developed specifically for neural networks, model-agnostic interpretation algorithms can be used for all types of machine learning models. Local Interpretable Model-agnostic Explanations (LIME) (Ribeiro et al., 2016) fits a linear model to synthetic instances that have randomly perturbed features in the vicinity of an instance. The obtained linear model is analyzed as a local surrogate of the original model to identify the important features for the prediction on this instance. Because this approach does not rely on gradient computation, LIME can be applied to any machine learning model, including non-differentiable models. Previously, we combined LIME and DeepLIFT to interpret a feedforward neural network model for predictive genomics (Badré et al., 2021). Kernel SHapley Additive explanations (SHAP) (Lundberg and Lec, 2017) uses a sampling method to find the Shapley value for each feature of a given input. The Multi-Objective Counterfactuals (MOC) method (Dandl et al., 2020) searches for the counterfactual explanations for an instance by solving a multi-objective optimization problem.

The importance scores calculated by the L2X algorithm (Chen et al., 2018) are based on the mutual information between the features and the output from a machine learning model. L2X is efficient because it approximates the mutual information using a variational approach. The second-order interpretation is more challenging than the first-order interpretation because d features would have d²−d/2 possible interactions to be evaluated. Computing the Hessian matrix of a model for the second-order interpretation is conceptually equivalent to, but much more computationally expensive than, computing the gradient for the first-order interpretation. Group Expected Hessian (GEH) (Cui et al., 2019) computes the Hessian of a Bayesian neural network for many regions in the input feature space and aggregates them to estimate an interaction score for every pair of features. The additive grooves algorithm (Sorokina et al., 2007) estimates the feature interaction scores by comparing the predictive performance of the decision tree containing all features with that of the decision trees with pairs of features removed. Neural Interaction Detection (NID) (Tsang et al., 2017) avoids the high computational cost of evaluating every feature pair by directly analyzing the weights in a feedforward neural network. If some features are strongly connected to a neuron in the first hidden layer and the paths from that neuron to the output have high aggregated weights, then NID considers these features to have strong interactions.

Model interpretations can be further classified as instance-wise interpretations or model-wise interpretations. Instance-wise interpretation algorithms, including Saliency (Simonyan et al., 2014), LIME (Ribeiro et al., 2016) and L2X (Chen et al., 2018), provide an explanation for a model's prediction for a specific instance. For example, an instancewise interpretation of a neural network model for predictive genomics may highlight the important SNPs in a specific subject which are the basis for the phenotype prediction of this subject. This is useful for intuitively assessing how well grounded the prediction of a model is for a specific subject. Model-wise interpretation provides insights into how a model makes predictions in general. CMV (Simonyan et al., 2014) was developed to interpret CNN models. Instance-wise interpretation methods can also be used to explain a model by averaging the explanations of all the instances in a test set. A model-wise interpretation of a predictive genomics model can reveal the important SNPs for a phenotype prediction in a large cohort of subjects. Model-wise interpretations shed light on the internal mechanisms of a machine learning model. In this study, we designed the LINA architecture and developed the first-order and second-order interpretation algorithms for LINA. The interpretation performance of the new methods was benchmarked using synthetic datasets and a predictive genomics application in comparison with state-of-the-art (SOTA) interpretation methods. The interpretations from LINA were more versatile and more accurate than those from the SOTA methods.

### Methods

### LINA Architecture

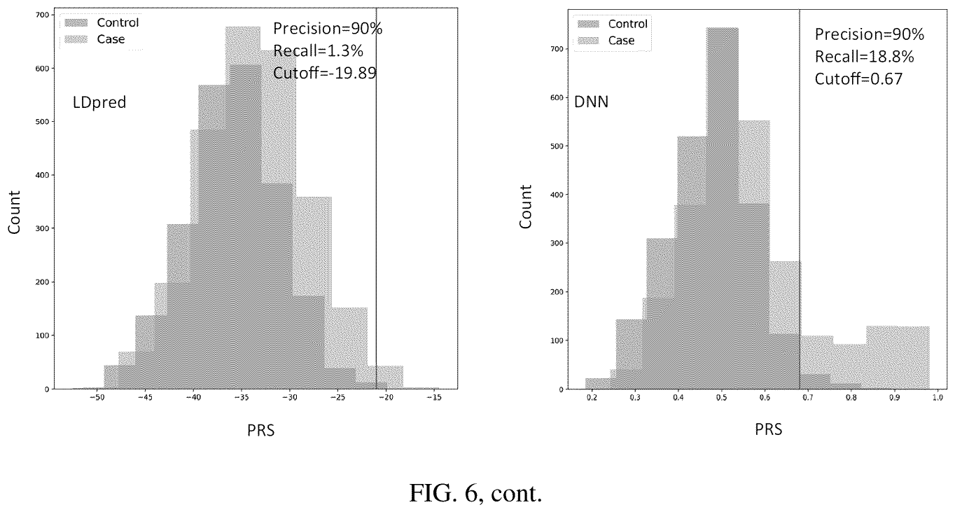

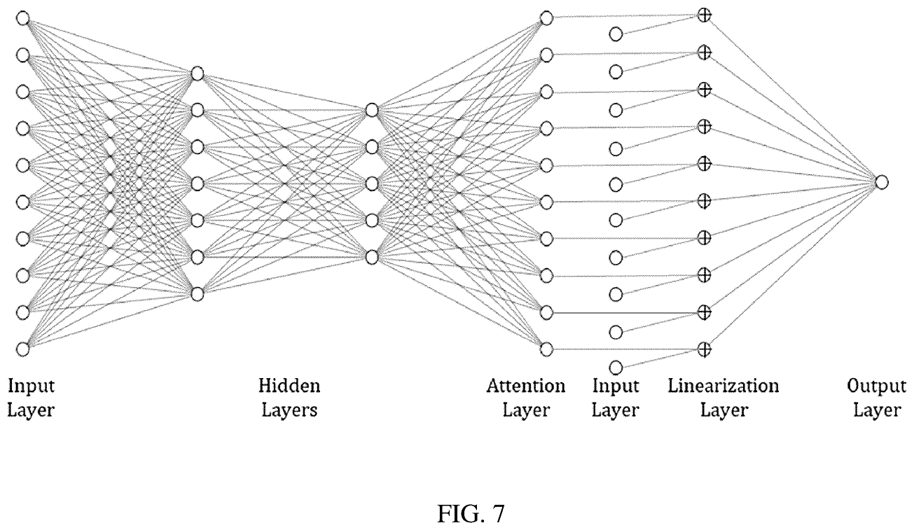

The key feature of the LINA architecture (FIG. 7) is the linearization layer, which computes the output as an element-wise multiplication product of the input features, attention weights, and coefficients:

$$y = S [ {K}^{T} ( A ∘ X ) + b ] = S ( {\sum }_{i = 1}^{d} ( {k}_{i} * {a}_{i} * {x}_{i} ) + b ) \qquad ( 3.1 )$$

where y is the output, X is the input feature vector, S ( ) is the activation function of the output layer, represents the element-wise multiplication operation, K and b are respectively the coefficient vector and bias that are constant for all instances, and A is the attention vector that adaptively scales the feature vector of an instance. X, A and K are three vectors of dimension d, which is the number of input features. The computation by the linearization layer and the output layer is also expressed in a scalar format in Equation 3.1. This formulation allows the LINA model to learn a linear function of the input feature vector, coefficient vector, and attention vector. The attention vector is computed from the input feature vector using a multi-layer neural network, referred to as the inner attention neural network in LINA. The inner attention neural network must be sufficiently deep for a prediction task owing to the designed low representational capacity of the remaining linearization layer in a LINA model. In the inner attention neural network, all hidden layers use a non-linear activation function, such as ReLU, but the attention layer uses a linear activation function to avoid any restriction in the range of the attention weights. This is different from the typical attention mechanism in existing attentional architectures which generally use the softmax activation function.

### The Loss Function

The loss function for LINA is composed of the training error loss, regularization penalty on the coefficient vector, and regularization penalty on the attention vector:

$$loss = E ( Y , {Y}_{t} rue ) + β * {‖ K ‖}_{2} + γ * {‖ A - 1 ‖}_{1} \qquad ( 3.2 )$$

where E is a differentiable convex training error function, ∥K∥l₂ is the L2 norm of the coefficient vector, ∥A−1∥₁ is the L1 norm of the attention vector minus 1, and β and γ are the regularization parameters. The coefficient regularization sets 0 to be the expected value of the prior distribution for K, which reflects the expectation of uninformative features. The attention regularization sets 1 to be the expected value of the prior distribution for A, which reflects the expectation of a neutral attention weight that does not scale the input feature. The values of β and γ and the choices of L2, L1, and L0 regularization for the coefficient and attention vectors are all hyperparameters that can be optimized for predictive performance on the validation set.
First-order interpretation

Interpretation from the gradient of the output, y, w.r.t the input feature vector, X. The output gradient can be decomposed as follows:

$$\frac{\partial y}{\partial {x}_{i}} = {k}_{i} * {a}_{i} + {\sum }_{j = 1}^{d} \frac{\partial {a}_{j}}{\partial {x}_{i}} * {x}_{j} \qquad ( 3.3 )$$

The decomposition of the output gradient in LINA shows that the contribution of a feature in an attentional architecture comprises (i) a direct contribution to the output weighted by its attention weight and (ii) an indirect contribution to the output during attention computation. This indicates that using attention weights directly as a measure of feature importance omits the indirect contribution of a feature in the attention mechanism. For the instance-wise first-order interpretation, we defined

$${FQ}_{i} = \frac{\partial y}{\partial {x}_{i}} \qquad ( 3.4 )$$

as the full importance score for feature i,

$$DQi = kiai \qquad ( 3.5 )$$

as the direct importance score for feature i, and

$${IQ}_{i} = {\sum }_{{j = 1}^{}}^{d} \frac{\partial {a}_{j}}{\partial {x}_{i}} {x}_{j} \qquad ( 3.6 )$$

as the indirect importance score for feature i. For the model-wise first-order interpretation, we defined the model-wise full importance score (FPᵢ), direct importance score (DPᵢ), and indirect importance score (IP;) for feature i as the averages of the absolute values of the corresponding instance-wise importance scores of this feature across all instances in the test set:

$${FP}_{i} = \vert {FQ}_{i} \vert \qquad ( 3.7 )$$

$${DP}_{i} = \vert {DQ}_{i} \vert \qquad ( 3.8 )$$

$${IP}_{i} = \vert {IQ}_{i} \vert \qquad ( 3.9 )$$

Because absolute values are used, the model-wise FPᵢ of feature i is no longer a sum of its IP; and DPᵢ.

Second-order interpretation

It is computationally expensive and unscalable to compute the Hessian matrix for a large LINA model. Here, the Hessian matrix of the output w.r.t. the input feature vector is reducible to the Jacobian matrix of the attention vector w.r.t. the input feature vector in a LINA model, which is computationally feasible to calculate when the network utilizes leaky-ReLU or ReLU activation function. It is derived as follows:

$$\frac{{\partial }^{2} y}{\partial {x}_{i} \partial {x}_{j}} = {k}_{j} \frac{\partial {a}_{j}}{\partial {x}_{i}} + {k}_{i} \frac{\partial {a}_{i}}{\partial {x}_{j}} \qquad ( 3.1 )$$

For any neuron, q, in the attention layer that outputs A (i.e., q∈A):

$$\frac{{\partial }^{2} {a}_{q}}{\partial {x}_{j} \partial {x}_{i}} = 0$$

For any neuron a E A:

$$aq = {\sum }_{i = 1}^{{m}_{l}} w q , k , lfk , l$$

$$\frac{\partial {a}_{q}}{\partial {x}_{j}} = {\sum }_{i = 1}^{{m}_{l}} {w}_{q , k , l} \frac{\partial {f}_{k , l}}{\partial {x}_{j}}$$

$$\frac{{\partial }^{2} {a}_{q}}{\partial {x}_{i} \partial {x}_{j}} = {\sum }_{i = 1}^{{m}_{l}} {w}_{q , k , l} \frac{{\partial }^{2} {f}_{k , l}}{\partial {x}_{i} \partial {x}_{j}}$$

where f_{k,l} is the activation function output from neuron k on hidden layer/containing mi neurons, and w(i,k,l) the coefficient of the connection between neuron q on layer A and neuron k on layer l. The K-weighted sum of the omitted second-order derivatives of the attention weights constitutes the approximation error. The performance of the second-order interpretation based on this approximation is benchmarked using synthetic and real-world datasets.

For instance-wise second-order interpretation, we define a directed importance score of feature r to feature c:

$${SQ}_{r} = {k}_{c} \frac{\partial {a}_{c}}{\partial {x}_{r}} \qquad ( 3.11 )$$

This measures the importance of feature r in the calculation of the attention weight of feature c. In other words, this second-order importance score measures the importance of feature r to the direct importance score of feature c for the output. For the model-wise second-order interpretation, we defined an undirected importance score between feature r and feature c based on their average instance-wise second-order importance score in the test set:

$${SP}_{c , r} = \vert {SQ}_{r}^{c} + S {Q}_{c}^{r} \vert \qquad ( 3.12 )$$

Recap of the LINA importance scores

The notations and definitions of all the importance scores for a LINA model are recapitulated below. FQ and SQ are selected as the first-order and second-order importance scores, respectively, for instance-wise interpretation. FP and SP are used as the firstorder and second-order importance scores, respectively, for model-wise interpretation.

### Data and Experimental Setup

California housing dataset

The California housing dataset (Pace and Barry, 1997) was used to formulate a simple regression task, which is the prediction of the median sale price of houses in a district based on eight input features (Table 6). The dataset contained 20640 instances (districts) for model training and testing.

TABLE 6
The linearization outputs and first-order instance-wise importance
scores for a district from the California housing dataset

| | Outputs | | | | | | |
|---|---|---|---|---|---|---|---|
| | Linearization Output | | | | First-order Instance-wise | | |
| | Coefficients | Attention | Features | Products | Importance Scores | | |
| Features | (K) | (A) | (X) | (KAX) | FQ | DQ | IQ |
| longitude | 249 | 221 | 0.22 | 11,932 | −51,296 | 55,108 | −106,404 |
| latitude | 257 | −299 | −0.56 | 42,700 | −211,275 | −76,933 | −134,343 |
| median_age | 213 | −275 | −1.35 | 79,230 | 33,407 | −58,524 | 91,930 |
| total_rooms | 173 | 158 | 1.32 | 36,024 | −17,957 | 27,230 | −45,187 |
| total_bedrooms | 184 | 240 | 1.10 | 48,531 | 5,614 | 44,281 | −38,667 |
| population | 200 | −19 | 1.54 | −5,690 | −62,220 | −3,695 | −58,525 |
| households | 189 | 233 | 1.20 | 52,532 | 32,443 | 43,951 | −11,508 |
| median_income | 174 | 125 | 0.91 | 19,777 | 73,337 | 21,736 | 51,601 |
| bias | | | | 149 | | | |
| median_house_price | | | | 285,183 | | | |

First-order benchmarking datasets

Five synthetic datasets, each containing 20,000 instances, were created using the sigmoid functions to simulate binary classification tasks. These functions were created following the examples in Chen et al., 2018 (Table 7) for the first-order interpretation benchmarking. All five datasets included ten input features.

TABLE 7

| Order | Target | Acronym | Definition |
|---|---|---|---|
| First- | Instance- | FQ | FQᵢ = DQᵢ + IQᵢ |
| order | wise | DQ | DQᵢ = kᵢaᵢ |
| | | IQ | IQᵢ = Σ_{c = 1}^{d} SQᵢ^{c} x_{c} |
| | Model- | FP | FPᵢ = \|̅F̅Q̅ᵢ̅\|̅ |
| | wise | DP | DPᵢ = \|̅D̅Q̅ᵢ̅\|̅ |
| | | IP | IPᵢ = \|̅I̅Q̅ᵢ̅\|̅ |
| Second- order | Instance- wise | SQ | ${SQ}_{r}^{c} = {k}_{c} \frac{\partial {a}_{c}}{\partial {x}_{r}}$ |
| | Model- | SP | SP_{c,r} = \|̅S̅Q̅ᵣ̅^̅{̅c̅}̅ +̅ S̅Q̅_̅{̅c̅}̅^̅{̅r̅}̅\|̅ |
| | wise | | |

The values of the input features were independently sampled from a standard Gaussian distribution:

$${x}_{i} ∼ N ( 0 , 1 ) , i ∈ \{ 1 , 2 , … , 10 \} .$$

The target value was set to 0 if the sigmoid function output is (0,0.5). The target value was set to 1, if the sigmoid function output is [0.5,1). We used the following five sigmoid functions of different subsets of the input features:

$$Sig ( 4 {X}_{1}^{2} - 3 {X}_{2}^{2} - 2 {X}_{3}^{2} + {X}_{4}^{2} ) . \qquad ( F 1 )$$

This function contains four important features with independent squared relationships with the target. The ground-truth rankings of the features by first-order importance are X₁, X₂, X₃, and X₄.

The remaining six uninformative features are tied in the last rank.

$$Sig ( - 10 sin ( {X}_{1} ) + 2 \vert {X}_{2} \vert + {X}_{3} - exp ( - {X}_{4} ) ) . \qquad ( F2 )$$

This function contains four important features with various non-linear additive relationships with the target. The ground-truth ranking of the features is X₁, X₄, X₂, and X₃. The remaining six uninformative features are tied in the last rank.

(F3): Sig (4X₁X₂X₃+X₄X₅X₆). This function contains six important features with multiplicative interactions among one another. The ground-truth ranking of the features is X₁, X₂, and X₃ tied in the first rank, X₄, X₅, and X₆ tied in the second rank, and the remaining uninformative features tied in the third rank.

(F4): Sig (−10 sin (X₁X₂X₃)+|X₄X₅X₆|). This function contains six important features with multiplicative interactions among one another and non-linear relationships with the target. The ground-truth ranking of the features is X₁, X₂, and X₃ tied in the first rank, X₄, X₅, and X₆ tied in the second rank, and the other four uninformative features tied in the third rank. (F5): Sig (−20 sin (X₁X₂)+21X₃₁+X₄X₅-4exp(−X₆)). This function contains six important features with a variety of non-linear relationships with the target. The ground-truth ranking of the features is X₁ and X₂ tied in the first rank, X₆ in the second, X₃ in the third, X₄ and X₅ tied in the fourth, and the remaining uninformative features tied in the fifth.

Second-order benchmarking dataset

Ten regression synthetic datasets, referred to as F6-A, F7-A, F8-A, F9-A, and F10-A (-A datasets) and F6-B, F7-B, F8-B, F9-B, and F10-B (-B datasets) were created. The -A datasets followed the examples in (Tsang et al., 2017) for the second-order interpretation benchmarking. The -B datasets used the same functions below to compute the target as the -A datasets, but included more uninformative features to benchmark the interpretation performance on high-dimensional data. Each -A dataset contained 5,000 instances. Each-B dataset contained 10,000 instances. The five -A datasets included 13 input features. The five -B datasets included 100 input features, some of which were used to compute the target. In F7-A/B, F8-A/B, F9-A/B, and F10-A/B, the values of the input features of an instance were independently sampled from a standard uniform distribution: Xᵢ˜U(−1,1),i∈{1,2, . . . , 13} in the -A datasets or i∈{1,2, . . . , 100} in the -B datasets. In the F6 dataset, the values of the input features of an instance were independently sampled from two uniform distributions: Xᵢ˜U (0,1), i∈ 1,2,3,6,7,9, 11, 12, 13} in the -A datasets and i∈{1,2,3,6, 7,9, 11, . . . , 100} in the -B datasets; and Xᵢ˜U(0.6, 1), i € {4,5,8,10} in both.

The value of the target for an instance was computed using the following five functions:

### (F6-A) and (F6-B)

$${π}^{{X}_{1} {X}_{2}} \sqrt{{X}_{3}} + {sin}^{- 1} {X}_{4} + log ( {X}_{3} + {X}_{5} ) + \frac{{X}_{0}}{{X}_{10}} \sqrt{\frac{{X}_{7}}{{X}_{8}}} - {X}_{2} {X}_{7} .$$

This function contains eleven pairwise feature interactions: {(X₁,X₂), (X₁,X₃), (X₂,X₃), (X₃,X₅), (X₇,X₈), (X₇,X₉), (X₇,X₁₀), (X₈,X₉), (X₈, X₁₀), (X₉,X₁₀), (X₂,X₇)}.

### (F7-A) and (F7-B):

$$exp ( \vert {X}_{1} - {X}_{2} \vert ) + \vert {X}_{2} {X}_{3} \vert - {X}_{3}^{2 \vert {X}_{4} \vert} + log ( {X}_{4}^{2} + {X}_{5}^{2} + {X}_{7}^{2} + {X}_{8}^{2} ) + {X}_{9} + \frac{{X}_{10}^{2}}{1 + {X}_{10}^{2}} .$$

This function contains nine pairwise interactions: {(X₁,X₂), (X₂,X₃), (X₃,X₄), (X₄,X₅), (X₄,X₇), (X₄,X₈), (X₅,X₇), (X₅,X₈), (X₇,X₈)}.

(F8-A) and (F8-B): sin (|X₁X₂|+1)−log (|X₃X₄₁₊₁)+cos (X₅+X₆−X₈)+\sqrt{X₈²+X₉²+X₁₀²}. This function contains ten pairwise interactions: {(X₁,X₂), (X₃,X₄), (X₅,X₆), (X₄,X₇), (X₅,X₆), (X₅,X₈), (X₆,X₈), (X₈,X₉), (X₈,X₁₀), (X₉,X₁₀)}.

(F9-A) and (F9-B):

$${tanh ( {X}_{1} {X}_{2} + {X}_{3} {X}_{4} )}^{p} \vert {X}_{5} \vert + log [ {( {X}_{6} {X}_{7} {X}_{8} )}^{2} + 1 ] + {X}_{9} {X}_{10} + \frac{1}{1 + \vert {X}_{10} \vert} .$$

This function contains thirteen pairwise interactions: {(X₁,X₂), (X₁,X₃), (X₂,X₃), (X₂,X₄), (X₃,X₄), (X₁,X₅), (X₂,X₅), (X₃,X₅), (X₄,X₅), (X₆,X₇), (X₆,X₈), (X₇,X₈), (X₉,X₁₀)}.

(F10-A) and (F10-B): cos (X₁X₂X₃)+sin (X₄X₅X₆). This function contains six pairwise interactions: {(X₁,X₂), (X₁,X₃), (X₂,X₃), (X₄,X₅), (X₄,X₆), (X₅,X₆)}.

Breast cancer dataset

The Discovery, Biology, and Risk of Inherited Variants in Breast Cancer (DRIVE) project (Amos et al., 2017) generated a breast cancer dataset (NIH dbGaP accession number: phs001265.v1.p1) for genome-wide association study (GWAS) and predictive genomics. This cohort contained 26,053 case subjects with malignant tumor or in situ tumor and 23,058 control subjects with no tumor. The task for predictive genomics is a binary classification of subjects between cases and controls. The breast cancer dataset was processed using PLINK (Purcell et al., 2007) as described previously (Badré et al.,2021) to compute the statistical significance of the SNPs. Out of a total of 528,620 SNPs, 1541 SNPs had a p-value lower than 10⁻⁶ and were used as the input features for predictive genomics. To benchmark the performance of the model interpretation, 1541 decoy SNPs were added as input features. The frequencies of homozygous minor alleles, heterozygous alleles, and homozygous dominant alleles were the same between decoy SNPs and real SNPs. Because decoy SNPs have random relationships with the case/control phenotype, they should not be selected as important features or be included in salient interactions by model interpretation.

### Implementations and Evaluation Strategies

The California Housing Dataset was partitioned into a training set (70%), a validation set (20%), and a test set (10%). The eight input features were longitude, latitude, median age, total rooms, total bedrooms, population, households, and median income. The median house value was the target of the regression. All the input features were standardized to zero mean and unit standard deviation based on the training set. Feature standardization is critical for model interpretation in this case because the scale for the importance scores of a feature is determined by the scale for the values of this feature, and comparison of the importance scores between features requires the values of the features to be in the same scale. The LINA model comprised an input layer (8 neurons), five fully connected hidden layers (7, 6, 5, 4, and 3 neurons), and an attention layer (8 neurons) for the inner attention neural network, followed by a second input layer (8 neurons), a linearization layer (8 neurons), and an output layer (1 neuron). The hidden layers used ReLU as the activation function. No regularization was applied to the coefficient vector and LI regularization was applied to the attention vector (y=10⁻⁶).

The LINA model was trained using the Adam optimizer with a learning rate of 10⁻². The predictive performance of the obtained LINA model was benchmarked to have an RMSE of 71055 in the test set. As a baseline model for comparison, a gradient boosting model achieved an RMSE of 77852 in the test set using 300 decision trees with a maximum depth of 5. For the first-order interpretation, each synthetic dataset was split into a cross-validation set (80%) for model training and hyperparameter optimization and a test set (20%) for performance benchmarking and model interpretation. A LINA model and a feedforward neural network (FNN) model were constructed using 10-fold cross-validation. For the first four synthetic datasets, the inner attention neural network in the LINA model had 3 layers containing 9 neurons in the first layer, 5 neurons in the second layer, and 10 neurons in the attention layer. The FNN had 3 hidden layers with the same number of neurons in each layer as the inner attention neural network in the LINA model.

For the fifth function with more complex relationships, the first and second layers were widened to 100 and 25 neurons, respectively, in both the FNN and LINA models to achieve a predictive performance similar to the other datasets in their respective validation sets. Both the FNN and LINA models were trained using the Adam optimizer. The learning rate was set to 10⁻². The mini-batch size was set to 32. No hyperparameter tuning was performed. The LINA model was trained with the L2 regularization on the coefficient vector (8=10⁻⁴) and the L1 regularization on the attention vector (y=10⁻⁶). The values of B and y were selected from 10⁻², 10⁻³, 10⁻⁴,10⁻⁵, 10⁻⁶, 10⁻⁷, and 0 based on the predictive performance of the LINA model on the validation set. Batch normalization was used for both architectures. Both the FNN and LINA models achieved predictive performance at approximately 99% AUC on the test set in the five first-order synthetic datasets, which was comparable to (Chen et al., 2018). Deep Lift (Shrikumar et al., 2017), LIME (Ribeiro et al., 2016), Grad*Input (Shrikumar et al., 2017), L2X (Chen et al., 2018) and Saliency (Simonyan et al., 2014) were used to interpret the FNN model and calculate the feature importance scores using their default configurations. FP, DP, and IP scores were used as the first-order importance scores for the LINA model.

We compared the performances of the first-order interpretation of LINA with DeepLIFT, LIME, GradInput, and L2X. The interpretation accuracy was measured using the Spearman rank correlation coefficient between the predicted ranking of features by their first-order importance and the ground-truth ranking. This metric was chosen because it encompasses both the selection and ranking of the important features. For the second-order interpretation benchmarking, each synthetic dataset was also split into a cross-validation set (80%) and a test set (20%). A LINA model, an FNN model for NID, and a Bayesian neural network (BNN) for GEH, as shown in (Cui et al., 2019), were constructed based on the neural network architecture used in (Tsang et al., 2017) using 10-fold cross-validation. The inner attention neural network in the LINA model uses 140 neurons in the first hidden layer, 100 neurons in the second hidden layer, 60 neurons in the third hidden layer, 20 neurons in the fourth hidden layer, and 13 neurons in the attention layer. The FNN model was composed of 4 hidden layers with the same number of neurons in each layer as LINA's inner attention neural network.

The BNN model uses the same architecture as that of the FNN model. The FNN, BNN, and LINA models were trained using the Adam optimizer with a learning rate of 10⁻³ and a mini-batch size of 32 for the -A datasets and 128 for the -B datasets. The LINA model was trained using L2 regularization on the coefficient vector (β=10⁻⁴) and the L1 regularization on the attention vector (γ=10⁻⁶) with batch normalization.

Hyperparameter tuning was performed as described above to optimize the predictive performance. The FNN and BNN models were trained using the default regularization parameters, as shown in (Cui et al., 2019), (Tsang et al., 2017). Batch normalization was used for LINA. The FNN, BNN, and LINA models all achieved R² scores of more than 0.99 on the test sets of the five-A datasets, as in the examples in (Tsang et al., 2017), while their R² scores ranged from 0.91 to 0.93 on the test set of the five highdimensional-B datasets.

Pairwise interactions in each dataset were identified from the BNN model using GEH (Cui et al., 2019), the FNN model using NID (Tsang et al., 2017), and the LINA model using the SP scores. For GEH, the number of clusters was set to the number of features and the number of iterations was set to 20. NID was run using its default configuration. For a dataset with m pairs of ground-truth interactions, the top-m pairs with the highest interaction scores were selected from each algorithm's interpretation output. The percentage of ground-truth interactions in the top-m predicted interactions (i.e., the precision) was used to benchmark the secondorder interpretation performance of the algorithms. For the breast cancer dataset, 49111 subjects in the breast cancer dataset were randomly divided into the training set (80%), validation set (10%), and test set (10%). The FNN model and the BNN model had 3 hidden layers with 1000, 250, and 50 neurons as described previously (Badré et al., 2021). The same hyperparameters were used in a previous study (Badré et al., 2021). The inner attention neural network in the LINA model also used 1000, 250, and 50 neurons before the attention layer.

All of these models had 3082 input neurons for 1541 real SNPs and 1541 decoy SNPs. B was set to 0.01 and y to 0, which were selected from 10⁻²,10⁻³,10⁻⁴,10⁻⁵,10⁻⁶, 10⁻⁷, and 0 based on the predictive performance of the LINA model on the validation set. Early stopping based on the validation AUC score was used during training. The FNN, BNN, and LINA models achieved a test AUC of 64.8%, 64.8%, and 64.7% on the test set, respectively, using both the 1541 real SNPs with p-values less than 10⁻⁶ and the 1541 decoy SNPs. The test AUCs of these models were lower than that of the FNN model in our previous study (Badré et al., 2021) at 67.4% using real 5,273 SNPs with p-values less than 10⁻³ as input. As the same FNN architecture design was used in the two studies, the reduction in the predictive performance in this study can be attributed to the use of more stringent p-value filtering to retain only real SNPs with a high likelihood of having a true association with the disease and the addition of decoy SNPs for benchmarking the interpretation performance. Deep Lift (Shrikumar et al., 2017), LIME (Ribeiro et al., 2016), Grad*Input (Shrikumar et al., 2017), L2X (Chen et al., 2018) and Saliency (Simonyan et al., 2014) were used to interpret the FNN model and calculate the feature importance scores using their default configurations. The FP score was used as the first-order importance score for the LINA model. After the SNPs were filtered at a given importance score threshold, the false discovery rate (FDR) was computed from the retained real and decoy SNPs above the threshold. The number of retained real SNPs was the total positive count for the FDR. The number of false positive hits (i.e., the number of unimportant real SNPs) within the retained real SNPs was estimated as the number of retained decoy SNPs. Thus, FDR was estimated by dividing the number of retained decoy SNPs by the number of retained real SNPs. An importance-scoresorted list of SNPs from each algorithm was filtered at an increasingly stringent score threshold until reaching the desired FDR level. The interpretation performance of an algorithm was measured by the number of top-ranked features filtered at 0.1%, 1%, and 5% FDR and the FDRs for the top-100 and top-200 SNPs ranked by an algorithm.

For the second-order interpretation, pairwise interactions were identified from the BNN model using GEH (Cui et al., 2019), from the FNN model using NID (Tsang et al., 2017), and from the LINA model using the SP scores. For GEH, the number of clusters was set to 20 and the number of iterations was set to 20. While LINA and NID used all 4,911 subjects in the test set and completed their computation within an hour, the GEH results were computed for only 1000 random subjects in the test set over 2 days because GEH would have taken approximately two months to complete the entire test set with its n2 computing cost where n is the number of subjects. NID was run using its default configuration in the FNN model. The interpretation accuracy was also measured by the numbers of top-ranked pairwise interactions detected at 0.1%, 1%, and 5% FDR and the FDRs for the top-1000 and top-2000 interaction pairs ranked by an algorithm. A SNP pair was considered to be false positive if one or both of the SNPs in a pair was a decoy.

### Results

Demonstration of LINA on a real-world application

In this section, we demonstrate LINA using the California housing dataset, which has been used in previous model interpretation studies for algorithm demonstration (Cui et al., 2019), (Tsang et al., 2017). Four types of interpretations from LINA were presented, including the instance-wise first-order interpretation, the instance-wise second-order interpretation, the model-wise first-order interpretation, and the modelwise second-order interpretation.

### Instance-wise Interpretation

Table 6 shows the prediction and interpretation results of the LINA model for an instance (district #20444) that had a true median price of $208600. The predicted price of $285183 was simply the sum of the eight element-wise products of the attention, coefficient, and feature columns plus the bias. This provided an easily understandable representation of the intermediate computation behind the prediction for this instance.

For example, the median age feature had a coefficient of 213 in the model. For this instance, the median age feature had an attention weight of −275, which switched the median age to a negative feature and amplified its direct effect on the predicted price in this district. The product of the attention weight and coefficient yielded the direct importance score of the median age feature (i.e., DQ=−58,524), which represented the strength of the local linear association between the median age feature and the predicted price for this instance. By assuming that the attention weights of this instance are fixed, one can expect a decrease of $58,524 in the predicted price for an increase in the median age by one standard deviation (12.28 years) for this district. But this did not consider the effects of the median age increase on the attention weights, which was accounted for by its indirect importance score (i.e., IQ=91,930). The positive IQ indicated that a higher median age would increase the attention weights of other positive features and increase the predicted price indirectly. Combining the DQ and IQ, the positive FQ of 33,407 marked the median age to be a significant positive feature for the predicted price, perhaps through the correlation with some desirable variables for this district. This example suggested a limitation of using the attention weights themselves to evaluate the importance of features in the attentional architectures. The full importance scores represented the total effect of a feature's change on the predicted price. For this instance, the latitude feature had the largest impact on the predicted price. Table 8 presents a second-order interpretation of the prediction for this instance. The median age row in Table 8 shows how the median age feature impacted the attention weights of the other features. The two large positive SQ values of median age to the latitude and longitude features indicated significant increases of the two location features' attention weights with the increase of the median age. In other words, the location becomes a more important determinant of the predicted price for districts with older houses. The total bedroom feature received a large positive attention weight for this instance. The total bedroom column in Table 8 shows that the longitude and latitude features are the two most important determinants for the attention weights of the total bedroom feature. This suggested how a location change may alter the direct importance of the total bedroom feature for the price prediction of this district.

TABLE 8
Second-order instance-wise importance scores of feature r (row r) to feature c (column c)

| | Column features (c) | | | | | | | |
|---|---|---|---|---|---|---|---|---|
| | | | median_{—} | total_{—} | total_{—} | | | median_{—} |
| Row features (r) | longitude | latitude | age | rooms | bedrooms | population | households | income |
| longitude | −17,234 | −33,983 | 19,682 | −10,797 | −9,572 | −13,375 | −1,153 | 4,899 |
| latitude | 22,696 | 44,572 | −25,631 | 13,068 | 12,002 | 18,119 | 1,035 | −10,005 |
| median_age | 18,591 | 18,555 | −14,262 | 7,140 | 5,749 | 8,328 | 2,586 | −8,357 |
| total_rooms | −13,249 | −17,930 | 11,547 | −4,102 | −4,198 | −8,626 | −526 | 12,029 |
| total_bedrooms | −16,973 | −19,799 | 14,110 | −7,173 | −5,943 | −8,597 | −2,123 | 7,328 |
| population | 932 | 11,223 | −4,307 | 1,052 | 1,947 | 4,842 | −1,471 | −4,623 |
| households | −18,646 | −25,227 | 16,879 | −8,943 | −7,507 | −10,514 | −2,163 | 6,829 |
| median_income | −8,713 | −20,704 | 10,829 | −3,928 | −4,515 | −9,182 | 758 | 9,546 |

### Model-wise Interpretation

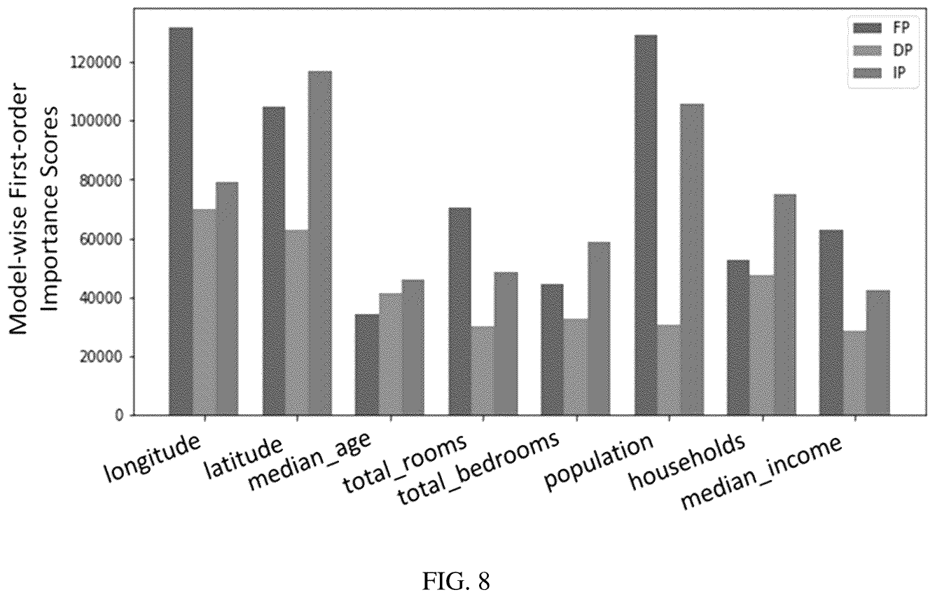

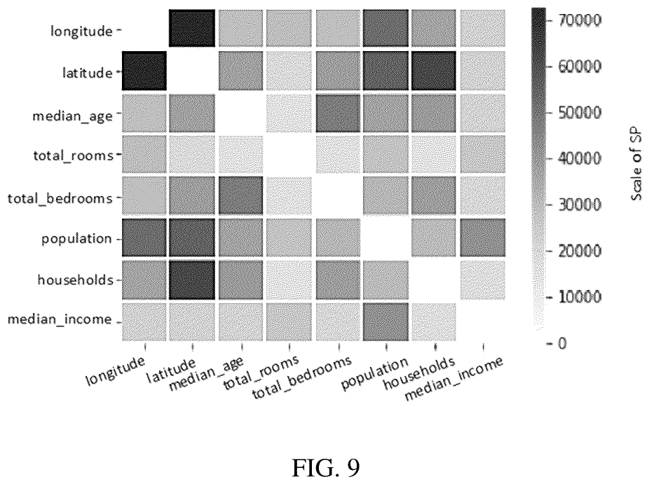

FIG. 8 shows the first-order model-wise interpretation results across districts in the California Housing dataset. The longitude, latitude, and population were the three most important features. The longitude and latitude had both high direct importance scores and high indirect importance scores. However, the population feature derived its importance mostly from its heavy influence on the attention weights as measured by its indirect importance score. FIG. 9 shows the second-order model-wise interpretation results for pairs of different features. Among all the feature pairs, the latitude and longitude features had the most prominent interactions, which was reasonable because the location was jointly determined by these two features. Some significant differences existed between the instance-wise interpretation and model-wise interpretation (e.g., Table 6 vs. FIG. 8 and Table 8 vs. FIG. 9). This illustrates the need for both instance-wise and model-wise interpretation methods for different purposes.

TABLE 9
Benchmarking of the first-order interpretation performance
using five synthetic datasets (F1 to F5)*

| | Datasets | | | | | |
|---|---|---|---|---|---|---|
| Methods | F1 | F2 | F3 | F4 | F5 | Average |
| LINA DP | 1.00 ± | 0.88 ± | 0.25 ± | 0.65 ± | 0.92 ± | 0.74 ± |
| | 0.00 | 0.03 | 0.07 | 0.05 | 0.03 | 0.04 |
| LINA IP | 1.00 ± | 0.92 ± | 0.69 ± | 0.84 ± | 0.96 ± | 0.88 ± |
| | 0.00 | 0.03 | 0.01 | 0.03 | 0.03 | 0.02 |
| LINA FP | 1.00 ± | 0.97 ± | 1.00 ± | 0.91 ± | 1.00 ± | 0.98 ± |
| | 0.00 | 0.02 | 0.00 | 0.04 | 0.00 | 0.01 |
| DeepLift | 0.99 ± | 1.00 ± | 0.95 ± | 0.83 ± | 1.00 ± | 0.95 ± |
| | 0.01 | 0.00 | 0.03 | 0.12 | 0.00 | 0.03 |
| Saliency | 1.00 ± | 0.90 ± | 1.00 ± | 0.76 ± | 1.00 ± | 0.93 ± |
| | 0.00 | 0.01 | 0.00 | 0.11 | 0.00 | 0.03 |
| Grad*Input | 1.00 ± | 1.00 ± | 0.85 ± | 0.78 ± | 1.00 ± | 0.93 ± |
| | 0.00 | 0.00 | 0.08 | 0.12 | 0.00 | 0.04 |
| L2X | 0.59 ± | 0.41 ± | 0.15 ± | 0.30 ± | 0.5 ± | 0.39 ± |
| | 0.06 | 0.07 | 0.11 | 0.08 | 0.03 | 0.07 |
| LIME | −0.72 ± | −0.52 ± | −0.14 ± | −0.57 ± | −0.3 ± | −0.45 ± |
| | 0.0 | 0.08 | 0.07 | 0.05 | 0.06 | 0.05 |

*The best Spearman correlation coefficient for each synthetic dataset is highlighted in bold

Benchmarking of the first-order and second-order interpretation using synthetic datasets

In real-world applications, the true importance of features for prediction cannot be determined with certainty and may vary among different models. Therefore, previous studies on model interpretation (Ribeiro et al., 2016), (Cui et al., 2019) benchmarked their interpretation performance using synthetic datasets with known ground-truth of feature importance. In this study, we also compared the interpretation performance of LINA with the SOTA methods using synthetic datasets created as in previous studies (Chen et al., 2018), (Tsang et al., 2017).

The performance of the first-order interpretation of LINA was compared with DeepLIFT, LIME, Grad*Input, and L2X (Table 9). The three first-order importance scores from LINA, including FP, DP, and IP, were tested. The DP score performed the worst among the three, especially in the F3 and F4 datasets which contained interactions among three features. This suggested the limitation of using attention weights as a measure of feature importance. The FP score provided the most accurate ranking among the three LINA scores because it accounted for the direct contribution of a feature and its indirect contribution through attention weights. The first-order importance scores were then compared among different algorithms. L2X and LIME distinguished many important features correctly from un-informative features, but their rankings of the important features were often inaccurate. The gradient-based methods produced mostly accurate rankings of the features based on their first-order importance. Their interpretation accuracy generally decreased in datasets containing interactions among more features. Among all the methods, the LINA FP scores provided the most accurate ranking of the features on average.

The performance of the second-order interpretation of LINA was compared with those of GEH and NID (Table 1). There were a total of 78 possible pairs of interactions among 13 features in each-A synthetic dataset and there were 4950 possible pairs of interactions among 100 features in each-B synthetic dataset. The precision from random guesses was only ˜12.8% on average in the -A datasets and less than 1% in the -B datasets. The three second-order algorithms all performed significantly better than the random guess. In the -A datasets, the average precision of LINA SP was ˜80%, which was ˜12% higher than that of NID and ˜29% higher than that of GEH. The addition of 87 un-informative features in the -B datasets reduced the average precision of LINA by ˜15%, that of NID by ˜13%, and that of GEH by ˜22%. In the -B datasets, the average precision of LINA SP was ˜65%, which was ˜9% higher than that of NID and ˜35% higher than that of GEH. This indicates that more accurate second-order interpretations can be obtained from the LINA models.

TABLE 10
Precision of the second-order interpretation by LINA SP,
NID and GEH in ten synthetic datasets (F6 to F10)*

| Total | | | | |
|---|---|---|---|---|
| Features | Datasets | NID | GEH | LINA SP |
| 13 features | F6-A | 44.5% ± 0.2% | 50.0% ± 0.2% | 61.8% ± 0.2% |
| | F7-A | 98.0% ± 0.1% | 41.0% ± 0.2% | 92.0% ± 0.1% |
| | F8-A | 80.6% ± 0.2% | 48.8% ± 0.4% | 85.0% ± 0.2% |
| | F9-A | 62.2% ± 0.4% | 41.4% ± 0.3% | 70.0 ± 0.3% |
| | F10-A | 56.7% ± 0.3% | 75.0% ± 0.5% | 91.7% ± 0.3% |
| | Average | 68.4% ± 0.2% | 51.2% ± 0.3% | 80.1 ± 0.2% |
| 100 features | F6-B | 51.8% ± 0.2% | 18.1% ± 1.0% | 52.7% ± 0.3% |
| | F7-B | 44.0% ± 0.2% | 28.8% ± 0.4% | 90.0% ± 0.0% |
| | F8-B | 76.3% ± 0.1% | 47.9% ± 0.2% | 80%.0 ± 0.3% |
| | F9-B | 40.0% ± 0.3% | 41.8% ± 0.2% | 51.7% ± 0.3% |
| | F10-B | 66.6% ± 0.0% | 10.4% ± 1.0% | 50.0% ± 0.1% |
| | Average | 55.7% ± 0.2% | 29.4% ± 0.6% | 64.9% ± 0.2% |

*The best precision for each dataset is highlighted in bold

Benchmarking of the first-order and second-order interpretation using a predictive genomics application

As the performance benchmarks in synthetic datasets may not reflect those in realworld applications, we engineered a real-world benchmark based on a breast cancer dataset for predictive genomics. While it was unknown which SNPs and which SNP interactions were truly important for phenotype prediction, the decoy SNPs added by us were truly unimportant. Moreover, a decoy SNP cannot have a true interaction, such as XOR or multiplication, with a real SNP to have a joint impact on the disease outcome. Thus, if a decoy SNP or an interaction with a decoy SNP is ranked by an algorithm as important, it should be considered a false positive detection. As the number of decoy SNPs was the same as the number of real SNPs, the false discovery rate can be estimated by assuming that an algorithm makes as many false positive detections from the decoy SNPs as from the real SNPs. This allowed us to compare the number of positive detections by an algorithm at certain FDR levels.

The first-order interpretation performance of LINA was compared with those of DeepLIFT, LIME, Grad*Input, and L2X (Table 11). At 0.1%, 1%, and 5% FDR, LINA identified more important SNPs than other algorithms. LINA also had the lowest FDRs for the top-100 and top-200 SNPs. The second-order interpretation performance of LINA was compared with those of NID and GEH (Table 12). At 0.1%, 1%, and 5% FDR, LINA identified more pairs of important SNP interactions than NID and GEH did. LINA had lower FDRs than the other algorithms for the top-1000 and top-2000 SNP pairs. Both L2X and GEH failed to output meaningful importance scores in this predictive genomics dataset. Because GEH needed to compute the full Hessian, it was also much more computationally expensive than the other algorithms.

TABLE 11
Performance benchmarking of the first-order
interpretation for predictive genomics

| Methods | LINA FP | Saliency | grad*Input | DeepLift | LIME | L2X |
|---|---|---|---|---|---|---|
| # SNPs at 0.1% FDR | 127 | 35 | 75 | 75 | 9 | 0 |
| # SNPs at 1% FDR | 158 | 35 | 88 | 85 | 9 | 0 |
| # SNPs at 5% FDR | 255 | 57 | 122 | 119 | 9 | 0 |
| FDR at top-100 SNP | 0.0% | 7.5% | 3.0% | 2.0% | 16.3% | N/A |
| FDR at top-200 SNP | 1.5% | 16.2% | 9.3% | 9.3% | 20.5% | N/A |

TABLE 12
Performance benchmarking of the second-order
interpretation for predictive genomics

| Methods | LINA SP | NID | GEH |
|---|---|---|---|
| # SNP pairs at 0.1% FDR | 583 | 415 | 0 |
| # SNP pairs at 1% FDR | 1040 | 504 | 0 |
| # SNP pairs at 5% FDR | 2887 | 810 | 0 |
| FDR at top-1000 SNP pairs | 0.9% | 10.5% | N/A |
| FDR at top-2000 SNP pairs | 3.0% | 31.8% | N/A |

The existing model interpretation algorithms and LINA can provide rankings of the features or feature interactions based on their importance scores at arbitrary scales. We demonstrated that decoy features can be used in real-world applications to set thresholds for first-order and second-order importance scores based on the FDRs of retained features and feature pairs. This provided an uncertainty quantification of the model interpretation results without knowing the ground-truth in real-world applications. The predictive genomics application provided a real-world test of the interpretation performance of these algorithms. In comparison with the synthetic datasets, the predictive genomics dataset was more challenging for model interpretation, because of the low predictive performance of the models and the large number of input features. For this real-world application, LINA was shown to provide better first-order and second-order interpretation performance than existing algorithms on a model-wise level. Furthermore, LINA can provide instance-wise interpretation to identify important SNP and SNP interactions for the prediction of individual subjects. Model interpretation is important for making biological discoveries from predictive models, because first-order interpretation can identify individual genes involved in a disease ((Rivandi et al., 2018; Romualdo Cardoso et al., 2022)), and second-order interpretation can uncover epistatic interactions among genes for a disease ((Shaker and Senousy, 2019; van de Haar et al., 2019)). These discoveries may provide new drug targets ((Wang et al., 2018; Gao et al., 2019; Gon calves et al., 2020)) and enable personalized formulation of treatment plans ((Wu et al., 2016; Zhao et al., 2021; Velasco-Ruiz et al., 2021)) for breast cancer.

In this work, we designed a new neural network architecture, referred to as LINA, for model interpretation. LINA uses a linearization layer on top of a deep inner attention neural network to generate a linear representation of model prediction. LINA provides the unique capability of offering both first-order and second-order interpretations and both instance-wise and model-wise interpretations. The interpretation performance of LINA was benchmarked to be higher than the existing algorithms on synthetic datasets and a predictive genomics dataset.

### III.

Explainable multi-task learning improves the parallel estimation of polygenic risk scores for many diseases through shared genetic basis

The PRS of a complex disease quantifies the genetic risk of an individual for this disease based on many genetic variants across the whole genome of the individual. The risk variants are generally selected based on this disease's GWAS, often using a relaxed statistical significance threshold. As noted above, a PRS can be estimated using a variety of statistical methods, including BLUP and LDPred. Statistical models of PRS have been built for breast cancer (Khera et al., 2018), colorectal cancer [(Thomas et al., 2020), (Gola et al., 2020), Type-2 diabetes (Ge et al., 2022), cardiovascular disease (Ye et al., 2021), and many other diseases. These statistical methods generally assume that the effects of risk variants on a phenotype are linear and independent. Recently, machine learning approaches free of these assumptions (Ho et al., 2019) have been used to estimate the PRS for breast cancer (Badré et al., 2021), blood pressure (Elgart et al., 2022), and schizophrenia (Bracher-Smith et al., 2022). However, the existing studies generally focused on constructing independent PRS models for individual diseases.

Many complex diseases share a substantial amount of common risk genetic determinants. Genome-wide cross-trait analyses have been performed between obesity and cardiovascular diseases (Zhuang et al., 2021), between thyroid and breast cancers (Sutton et al., 2022), between uterine leiomyoma and breast cancer (Wu et al., 2022), between asthma and cardiovascular diseases (Zhou et al., 2022), between Alzheimer's disease and gastrointestinal tract disorders (Adewuyi et al., 2022), between Alzheimer disease and major depressive disorder (Lutz et al., 2020), between lung cancer and chronic bronchitis (Byun et al., 2021), and so on. These studies were often motivated by frequent co-occurrences of a pair of diseases in a population. Some of the epidemiological associations have been attributed to the shared genetic architecture between the diseases. The related genetic etiology among diseases can be caused by dysfunctions in some common enzymes or pathways, which may increase the clinical risks for multiple diseases directly or indirectly.

In the present work, it was hypothesized that shared genetic determinants among diseases can be exploited to improve their PRS estimation. This hypothesis was tested using a pandisease multi-task learning (MTL) approach (Caruana, 1998) based on an interpretable neural network architecture (Badré and Pan, 2022). MTL has been widely used in many computer vision (Girshick, 2015) and natural text processing (Liu et al., 2016) applications, in which the training examples have multiple labels to be predicted from the same input feature vectors. Unlike single-task learning (STL), which trains a model to predict each individual label independently, MTL trains a model to predict all labels in parallel. MTL has been shown to provide better predictive performance than STL when the learning tasks are related (Standley et al., 2019). Related tasks can enable a MTL model to learn a better-shared representation through data amplification, feature selection, regularization, and other beneficial effects (Fifty et al., 2021). However, if the tasks are unrelated, the predictive performance of MTL may be worse than that of STL, owing to the negative knowledge transfer among the tasks (Standley et al., 2019). Thus, if the hypothesis was invalid, the PRS learned for a disease in conjunction with other diseases by a pan-disease MTL model would be less accurate than the PRS learned for this disease by an STL model.

### Methods

Preparation of the phenotypic and genomic data

A total of 488,175 subjects were extracted from the UK Biobank dataset release version 2 (Bycroft et al., 2018). The phenotypic traits of the subjects were determined using the protocol and software described in a previous study (DeBoever et al., 2020). The diseases in subjects were identified using hospital inpatient records (ICD10 codes, UK Biobank Data Coding 19) and self-reported disease status (UK Biobank Data Coding 3 for cancers and UK Biobank Data Coding 6 for non-cancer diseases). The UKB genomic data covered a total of 805,426 SNPs. The genotypes of SNPs were encoded as 0 for homozygous with the minor allele, 1 for heterozygous alleles, or 2 for homozygous with the dominant allele. All the code for data processing, model training, performance benchmarking, and model interpretation is available publicly at https://github.com/thepanlab/GattacaNet2.

Construction of the MTL and STL models

The output of MTL LINA is a d ×1 vector, Y, containing the predicted states of d traits. The input of MTL LINA is an m x 1 vector, X, containing the genotypes of m SNPs. In this study, d=69 in the pan-cancer MTL model, d=362 in the pan-disease MTL model, and m=805426 in both models. MTL LINA can be expressed as:

$$y = S ( K \cdot ( A ⊙ X ) + B ) \qquad ( 4.1 )$$

$$A = F ( X )$$

where S ( ) was a sigmoid activation function to be applied element-wise to its input column vector, K was a dm coefficient matrix, A was a mx1 attention vector, B was a dₓ1 bias vector, · represented the matrix-vector multiplication, and (represented the element-wise multiplication. A was computed from X by a feedforward neural network, F ( ) composed of 3 hidden layers containing 1000, 250, and 50 neurons. A leaky-ReLU activation function, dropout with a dropout rate of 50%, and batch normalization were used in all three hidden layers. A linear activation function was used in the attention layer. K, B, and F ( ) were all learned from the training data. The loss function of MTL LINA was defined as:

$$loss = {W}^{T} E + β {‖ K ‖}_{2} \qquad ( 4.2 )$$

where W was a dₓ1 vector of the loss weights for all traits, E was a d1 vector of the cross-entropy losses for all traits, and ∥K∥₂ was the L2 norm of the coefficient matrix, and β was the regularization weight. In this study, W=[1, . . . , 1] T and β=10⁻³.

A total of 77 STL models were constructed for the 17 cancers and 60 non-cancer diseases with prevalence levels over 0.5%. All STL models used a feedforward neural network composed of three hidden layers containing 1000, 250, and 50 neurons as described previously (Badré et al., 2021). A leaky-ReLU activation function, dropout with a dropout rate of 50%, and batch normalization were also used in all three hidden layers. The cross-entropy loss function was used to train the STL models.

Training and benchmarking of the MTL and STL models

The 488,175 UKB subjects were randomly divided into a training set (70%), a validation set (15%), and a test set (15%). The training set was used to train all MTL and STL models by stochastic gradient descent. The training used mini-batches with a batch size of 512 and the Adam optimizer with an initial learning rate of 10⁻⁴. All MTL and STL models were trained for 100 epochs with checkpointing after every epoch. The checkpoints with the best performance on the validation set were kept for all MTL and STL models, which were the epoch-27 checkpoint for the pan-cancer MTL model and the epoch-25 checkpoint for the pan-disease MTL model. The training was carried out on a computer node with dual A100 40 GB GPUs and 256 GB system memory. The training data was lazy-loaded to minimize memory usage using the pandas plink (noa) library. After the training was completed, the predictive performances of all MTL and STL models were benchmarked using the test set.

Interpretation of the MTL models

The first-order model-wise LINA interpretation algorithm, as detailed in Equation 3.3 and the score FP (Equation 3.7), was used to identify important features (Badré and Pan, 2022) for each phenotype. A synthetic genomic vector was constructed for each subject to estimate the false discovery rate of the model interpretation, as shown previously (Badré and Pan, 2022). The synthetic genomic vectors of all subjects contained all their real SNPs and an equal number of decoy SNPs. The genotypes of the decoy SNPs were randomly set to be 0, 1, or 2 with the same probabilities observed in the real SNPs. Thus, the decoy SNPs had identical frequencies of homozygous minor alleles, heterozygous alleles, and homozygous dominant alleles as the real SNPs. But, because the decoy SNPs should have no association with the phenotypes, any decoy SNP identified as important by the interpretation algorithm was considered a false positive hit.

A pan-cancer MTL model was constructed and trained as described above using the synthetic genomic vectors of the subjects in the training set. The importance scores of both real and decoy SNPs were computed for each cancer using the subjects in the test set. Only SNPs on the non-sex chromosomes were considered for model interpretation. The FDR for an importance score threshold was estimated as the ratio between the numbers of decoy SNPs to real SNPs above this threshold. The important SNPs at 0.1% FDR and 5% FDR were identified for all cancers with >0.5% prevalence in the pan-cancer MTL model. The intersection and union of the important SNPs were counted between every pair of prevalent cancers. The genetic correlation between two cancers was computed as the Spearman correlation coefficient between the importance scores of the SNPs belonging to the union of the SNP sets of the two cancers at 5% FDR.

### Results

Parallel prediction of many diseases by MTL

A neural network architecture was developed to predict many traits of an individual from their whole genome (FIG. 10). This was an MTL extension of the linearizing neural network architecture (LINA) previously shown to provide good predictive performance for STL estimation of breast cancer PRS (Badré and Pan, 2022). All the traits shared a latent genomic representation, which was an element-wise multiplication of a learned attention vector and the input whole-genome vector. Each trait was predicted from the shared representation via a task-specific hidden layer. The output from the MTL model was a vector of character states for all the considered phenotypes, referred to as a whole-phenome vector.

Training a MTL model required a cohort of subjects with phenome-wide trait data. In this study, we used the United Kingdom Biobank (UKB) dataset and extracted 362 disease traits, including 69 cancer traits, from the electronic medical record of 488,175 UKB participants. 77 diseases, including 17 types of cancers and 60 non-cancer diseases, had prevalence levels higher than 0.5% in the UKB cohort. We constructed two MTL models, one to predict the 69 cancers (pan-cancer MTL) and the other one to predict all 362 diseases (pan-disease MTL). Instead of selecting SNPs for each disease based on their statistical association, we included all 805,426 SNPs genotyped in the UKB cohort as the input for both MTL models. The UKB cohort was randomly divided into a training set (70%) for model training, a validation set (15%) for hyperparameter optimization, and a test set (15%) for performance benchmarking. A model's training took approximately 5 days on a computer node with dual A100 40 GB GPUs. All the benchmarking results described below were based on the test set.

Improved accuracy for PRS estimation by MTL

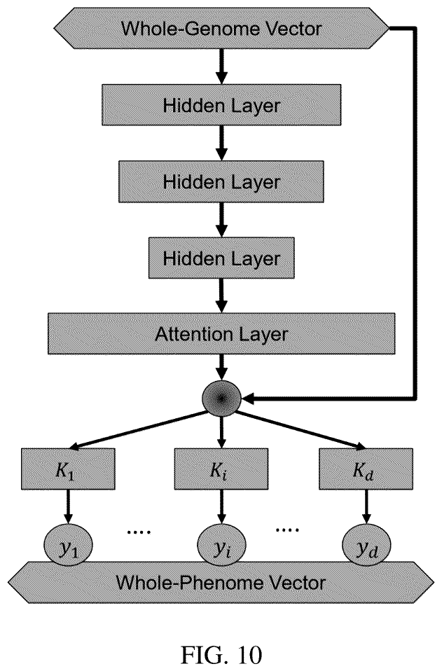

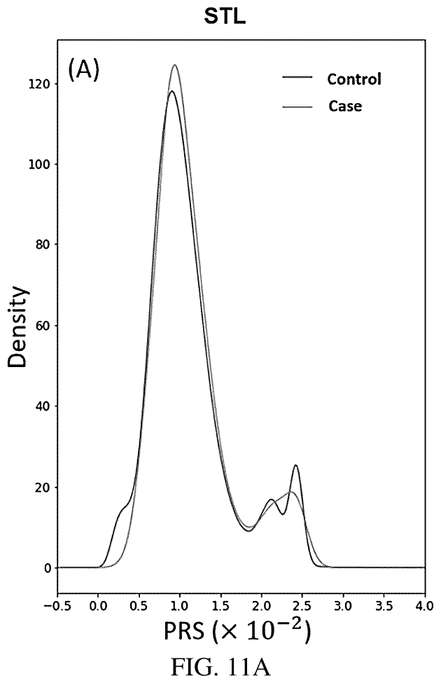

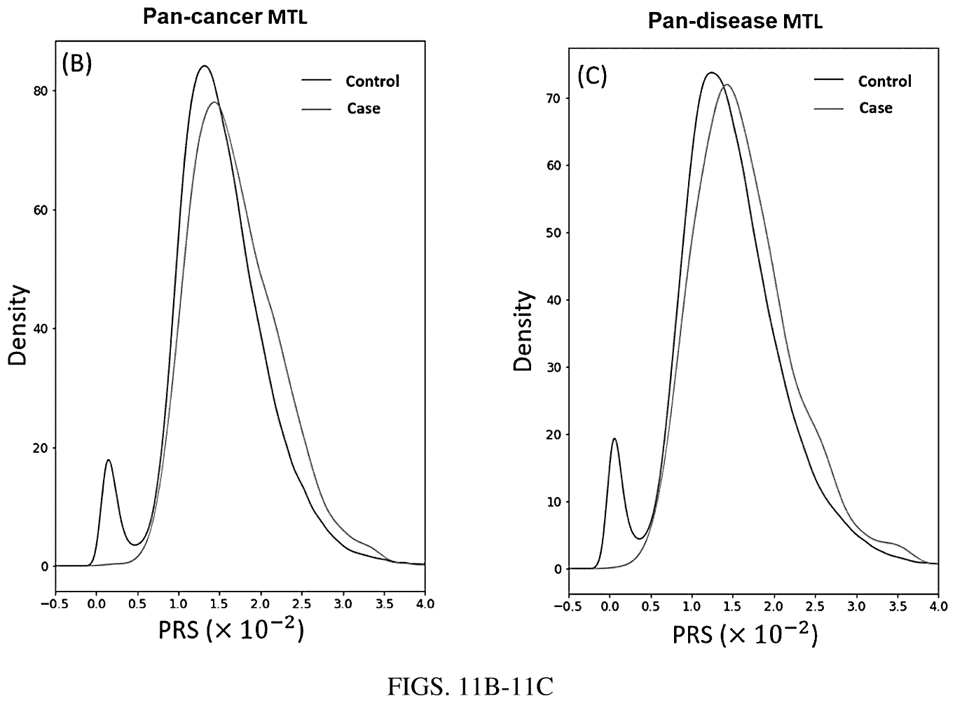

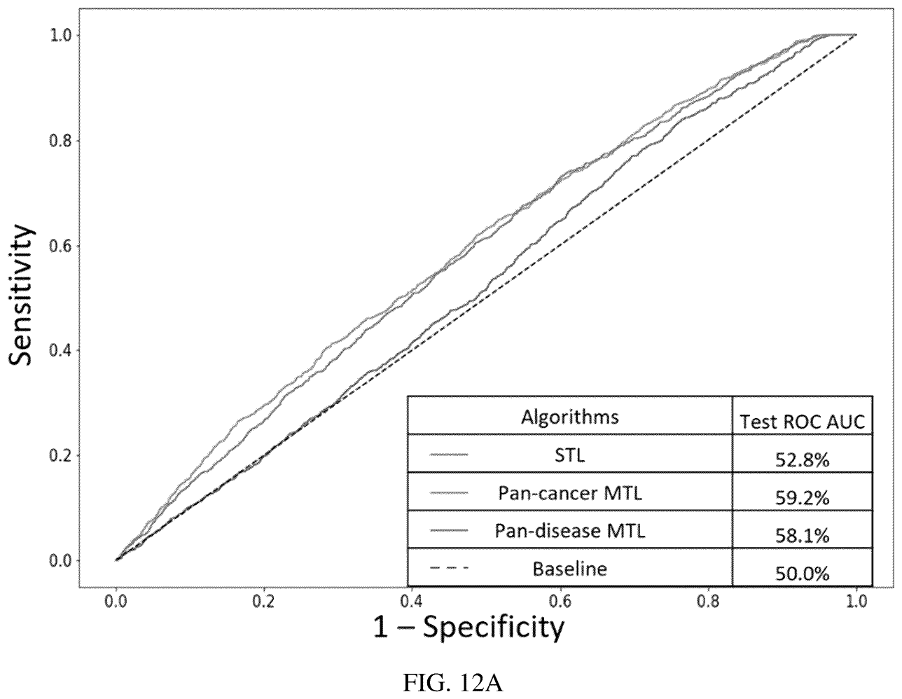

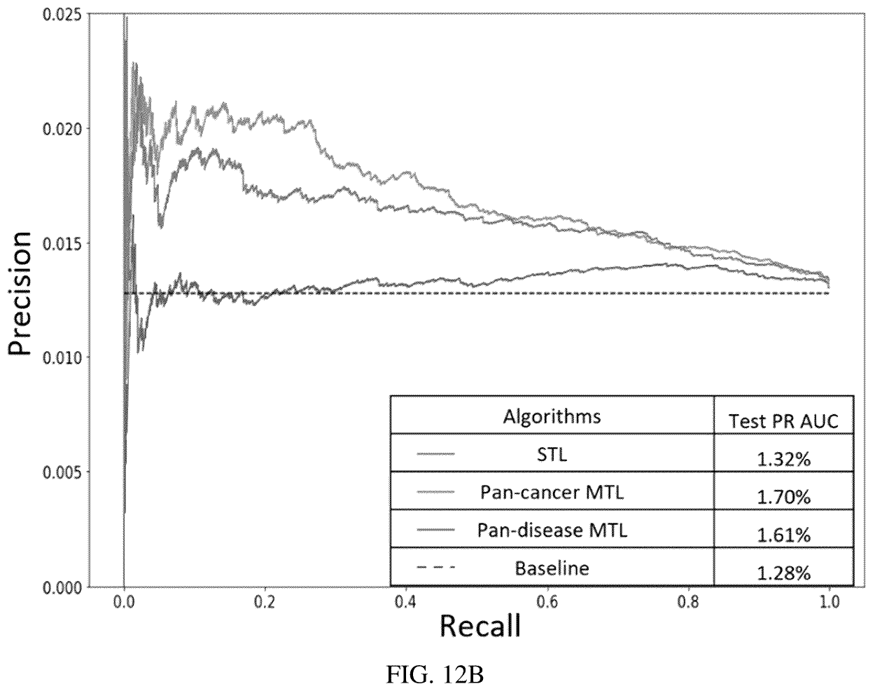

The estimation accuracy of malignant melanoma PRS was compared among STL, pancancer MTL, and pan-disease MTL (FIGS. 11-12). The same training data was used to train the STL model to predict malignant melanoma only (FIG. 11A), the pan-cancer MTL model to predict 69 cancers, including malignant melanoma (FIG. 11B), and the pan-disease MTL to predict malignant melanoma along with 361 other diseases (FIG. 11C). The MTL and STL models generated different distributions of PRS in the test set. The differences were especially pronounced on the two shoulders of the distributions, which represented the subjects with higher or lower genetic risks than the average. The separation between the PRS distribution of the control subjects and the PRS distribution of the case subjects was greater in the two MTL models than the STL model. The predictive performances of the three models were compared using the Receiver Operating Characteristics (ROC) curves (FIG. 12A). The Area Under the Curve (AUC) of the ROC curve by STL was only 2.8% higher than the 50% baseline, while those by the pan-cancer MTL and pan-disease MTL were 9.2% and 8.1% higher, respectively. Because of the imbalanced data, the predictive performances of the three models were also compared using the Precision-Recall (PR) curves (FIG. 12B). Both MTL models achieved much higher precisions at the same recall level than the STL model. The baseline of the PR curve was determined by the prevalence level of malignant melanoma in the UKB cohort, which was 1.28%. The PR AUC by STL was 0.04% higher than the baseline, while those by the pan-cancer MTL and pan-disease MTL were 0.42% and 0.33% higher, respectively. Overall, these metrics reflected better predictive performance of the two MTL models than the STL model for malignant melanoma.

The predictive performances of the two MTL models were then compared with the disease-specific STL models across 17 common cancers with prevalence levels higher than 0.5% (Table 13). The comparisons were made using both ROC AUC and PR AUC to account for the sensitivity, specificity, precision, and recall of the models. The two MTL models offered higher ROC AUC for 16 cancers and higher PR AUC for all 17 cancers than the disease-specific STL models. The magnitude of the performance improvement was quantified using the relative increase of the over-the-baseline AUC gain by an MTL model in comparison with the corresponding STL model. The average relative increase of ROC AUC over STL was 141% for the pan-cancer MTL and 153% for the pan-disease MTL. The average relative increase of PR AUC over STL was 96% for the pan-cancer MTL and 83% for the pan-disease MTL. The variability of the relative increases among different cancers suggested that each disease benefited to a different extent from MTL. The pan-cancer MTL had the highest ROC AUC for 4 cancers and highest PR AUC for 5 cancers. The pan-disease MTL had the highest ROC AUC for 12 cancer types and highest PR AUC for 12 cancer types. This suggested that the performance improvement from transfer learning increased with the number of traits in MTL. To further check if the performance gain by MTL over STL can be generalized across non-cancer diseases, we compared the pan-disease MTL model with the disease-specific STL models for 60 non-cancer diseases with prevalence levels higher than 0.5% (Table 14). The same set of performance metrics was used for the comparison. Compared with the disease-specific STL models, the pan-disease MTL model provided higher ROC AUC for 55 non-cancer diseases and higher PR AUC for 50 non-cancer diseases. The average relative increase by MTL across the 60 non-cancer diseases was 68% for ROC AUC and 82% for PR AUC. The benchmarking results for both cancer and non-cancer diseases indicated significant performance improvements by MTL over STL across many diseases.

TABLE 13
Comparison of STL, pan-cancer MTL, and pan-disease MTL by ROC
AUC and PR AUC for 17 cancer types with >0.5% prevalence

| | Receiver operating characteristics (ROC) AUC^{#} | | | | | Precision-recall (PR) AUC^{#} | | | | | |
|---|---|---|---|---|---|---|---|---|---|---|---|
| | STL | Pan-cancer MTL | | Pan-disease MTL | | STL | Pan-cancer MTL | | Pan-disease MTL | | |
| | ROC | ROC | Relative | ROC | Relative | PR | PR | Relative | PR | Relative | Prevalence |
| Diseases | AUC | AUC | increase* | AUC | increase* | AUC | AUC | increase* | AUC | increase* | (Baseline) |
| Malignant | 52.80% | 59.20% | 234% | 58.10% | 194% | 1.33% | 1.70% | 790% | 1.61% | 593% | 1.28% |
| melanoma | | | | | | | | | | | |
| Non-melanoma | 61.50% | 62.40% | 8% | 62.90% | 12% | 9.76% | 10.03% | 8% | 10.21% | 14% | 6.65% |
| skin cancer | | | | | | | | | | | |
| Skin cancer | 61.00% | 61.80% | 7% | 61.90% | 8% | 10.40% | 10.73% | 11% | 10.86% | 15% | 7.32% |
| Lung cancer | 59.10% | 60.30% | 14% | 60.50% | 16% | 1.39% | 1.44% | 11% | 1.51% | 23% | 0.90% |
| Intrathoracic | 59.10% | 60.70% | 18% | 61.00% | 21% | 1.54% | 1.58% | 7% | 1.65% | 20% | 1.01% |
| cancer | | | | | | | | | | | |
| Colorectal | 54.40% | 56.40% | 46% | 57.10% | 60% | 2.00% | 2.21% | 71% | 2.29% | 100% | 1.72% |
| cancer | | | | | | | | | | | |
| Colon cancer | 53.90% | 55.70% | 47% | 56.10% | 59% | 1.38% | 1.49% | 51% | 1.49% | 53% | 1.17% |
| Rectal cancer | 54.70% | 57.90% | 69% | 59.30% | 100% | 0.77% | 0.88% | 98% | 0.89% | 116% | 0.67% |
| Bladder cancer | 64.50% | 67.90% | 24% | 68.40% | 27% | 0.80% | 0.87% | 24% | 0.92% | 42% | 0.51% |
| Uterine cancer | 51.20% | 53.20% | 177% | 51.80% | 50% | 1.08% | 1.18% | 224% | 1.10% | 49% | 1.04% |
| Cervical cancer | 55.20% | 55.40% | 4% | 56.50% | 24% | 1.80% | 1.88% | 35% | 1.97% | 76% | 1.58% |
| Prostate cancer | 60.00% | 59.70% | −3% | 59.60% | −4% | 8.33% | 8.53% | 9% | 8.37% | 2% | 6.06% |
| Breast cancer | 57.00% | 58.30% | 19% | 58.10% | 16% | 9.38% | 9.67% | 13% | 9.79% | 20% | 7.25% |
| Female genital | 54.00% | 54.30% | 7% | 54.50% | 11% | 3.15% | 3.27% | 41% | 3.39% | 84% | 2.86% |
| tract cancer | | | | | | | | | | | |
| Male genital | 53.60% | 56.10% | 68% | 54.50% | 24% | 2.57% | 2.73% | 57% | 2.59% | 9% | 2.28% |
| tract cancer | | | | | | | | | | | |
| Lymphoma | 50.40% | 56.80% | 1442% | 57.90% | 1704% | 0.73% | 0.82% | 102% | 0.82% | 98% | 0.64% |
| Non-hodgkins | 52.10% | 56.60% | 220% | 57.80% | 278% | 0.61% | 0.68% | 81% | 0.69% | 95% | 0.53% |
| lymphoma | | | | | | | | | | | |

^{#}Best AUC highlighted in bold
*Relative increase = (MTL AUC − baseline AUC) − (STL AUC − baseline AUG) × 100%

TABLE 14
Comparison of STL and pan-disease MTL by ROC AUC and PR
AUC for 60 non-cancer diseases with >0.5% prevalence

| | Receiver operating | | | | | | |
|---|---|---|---|---|---|---|---|
| | characteristics (ROC) AUC^{#} | | | Precision-recall (PR) AUC^{#} | | | |
| | | Pan-disease | | | Pan-disease | | |
| | | MTL ROC | | | MTL PR | | |
| | | ROC | Relative | | PR | Relative | Prevalence |
| Disease | STL | AUC | increase* | STL | AUC | increase* | (Baseline) |
| Allergy or anaphylactic | 54.35% | 55.52% | 26.80% | 0.61% | 0.64% | 44.67% | 0.54% |
| reaction to food | | | | | | | |
| Angina | 64.08% | 65.33% | 8.88% | 5.52% | 5.60% | 3.59% | 3.23% |
| Appendicitis | 51.16% | 53.10% | 167.60% | 1.44% | 1.47% | 29.03% | 1.32% |
| Arthritis (nos) | 55.17% | 55.64% | 9.02% | 1.51% | 1.52% | 6.25% | 1.22% |
| Asthma | 60.01% | 61.15% | 11.39% | 16.44% | 17.02% | 12.37% | 11.75% |
| Atrial fibrillation | 63.69% | 64.62% | 6.78% | 4.12% | 4.42% | 18.82% | 2.50% |
| Atrial flutter | 64.05% | 64.69% | 4.59% | 3.60% | 3.70% | 6.62% | 2.12% |
| Back pain | 52.75% | 53.44% | 24.94% | 1.15% | 1.19% | 34.87% | 1.03% |
| Benign breast lump | 53.39% | 54.18% | 23.45% | 1.58% | 1.62% | 23.64% | 1.41% |
| Bronchitis | 51.87% | 52.95% | 57.92% | 1.32% | 1.33% | 9.67% | 1.21% |
| Cataract | 52.81% | 53.43% | 22.13% | 2.38% | 2.45% | 29.45% | 2.13% |
| Cervical spondylosis | 56.00% | 58.97% | 49.47% | 0.87% | 1.02% | 95.82% | 0.72% |
| Chickenpox | 53.73% | 54.16% | 11.41% | 2.58% | 2.62% | 20.03% | 2.33% |
| Gallstones | 63.43% | 64.95% | 11.36% | 2.65% | 2.86% | 23.82% | 1.75% |
| Chronic sinusitis | 53.23% | 53.92% | 21.31% | 0.82% | 1.07% | 343.92% | 0.75% |
| Depression | 57.35% | 58.46% | 15.15% | 7.93% | 8.29% | 20.87% | 6.24% |
| Diabetes | 67.26% | 67.92% | 3.83% | 9.75% | 9.99% | 4.78% | 4.59% |
| Dyspepsia/indigestion | 53.67% | 55.05% | 37.66% | 3.13% | 3.34% | 64.05% | 2.80% |
| Ear/vestibular disorder | 51.57% | 53.16% | 101.20% | 1.01% | 1.00% | −7.92% | 0.92% |
| Eczema/dermatitis | 54.62% | 56.98% | 51.32% | 3.54% | 3.93% | 80.88% | 3.07% |
| Emphysema/chronic | 58.26% | 60.08% | 22.08% | 1.94% | 2.03% | 14.61% | 1.35% |
| bronchitis | | | | | | | |
| Endometriosis | 54.13% | 53.80% | −7.88% | 1.77% | 1.75% | −9.67% | 1.58% |
| Epilepsy | 49.81% | 53.94% | 2188.46% | 0.81% | 0.93% | 1638.72% | 0.82% |
| Essential hypertension | 51.92% | 54.37% | 127.01% | 1.38% | 1.47% | 78.20% | 1.26% |
| Eye/eyelid problem | 49.52% | 50.96% | 298.50% | 0.79% | 0.84% | 711.13% | 0.80% |
| Fracture lower leg/ankle | 53.99% | 54.65% | 16.63% | 0.56% | 0.60% | 89.87% | 0.52% |
| Gastro-oesophageal reflux | 53.91% | 55.18% | 32.31% | 5.57% | 5.73% | 22.40% | 4.87% |
| (gord)/gastric reflux | | | | | | | |
| Glaucoma | 58.48% | 59.31% | 9.86% | 1.65% | 1.83% | 42.36% | 1.24% |
| Gout | 61.60% | 63.60% | 17.21% | 4.66% | 5.32% | 43.41% | 3.16% |
| Hayfever/allergic rhinitis | 55.02% | 57.16% | 42.54% | 7.62% | 8.13% | 44.51% | 6.48% |
| Heart attack (MI) | 71.25% | 72.56% | 6.16% | 5.06% | 5.27% | 7.30% | 2.30% |
| Hiatus hernia | 55.90% | 57.71% | 30.64% | 3.06% | 3.26% | 36.16% | 2.51% |
| Hypertension | 62.89% | 61.78% | −8.64% | 37.70% | 36.41% | −12.40% | 27.32% |
| Hyperthyroidism/ | 67.77% | 71.55% | 21.27% | 1.74% | 2.14% | 45.39% | 0.85% |
| thyrotoxicosis | | | | | | | |
| Hypothyroidism/ | 73.90% | 74.96% | 4.43% | 12.99% | 13.59% | 7.53% | 5.14% |
| myxoedema | | | | | | | |
| Inguinal hernia | 75.09% | 76.31% | 4.89% | 1.49% | 1.57% | 9.41% | 0.66% |
| Iron deficiency anaemia | 67.23% | 67.34% | 0.65% | 1.53% | 1.61% | 10.74% | 0.75% |
| Irritable bowel syndrome | 62.16% | 62.38% | 1.78% | 3.85% | 3.81% | −2.77% | 2.56% |
| Joint pain | 50.89% | 51.78% | 100.28% | 0.61% | 0.69% | 468.36% | 0.59% |
| Kidney stone/ureter stone/ | 60.69% | 65.07% | 41.01% | 1.29% | 1.53% | 52.22% | 0.84% |
| bladder stone | | | | | | | |
| Measles/morbillivirus | 54.81% | 53.73% | −22.39% | 2.06% | 1.97% | −34.87% | 1.78% |
| Migraine | 64.51% | 64.65% | 0.99% | 5.48% | 5.57% | 3.91% | 3.38% |
| Mumps/epidemic parotitis | 55.02% | 55.73% | 14.19% | 1.23% | 1.33% | 85.88% | 1.11% |
| Oesophagitis/barretts | 54.94% | 55.57% | 12.66% | 2.57% | 2.60% | 6.79% | 2.16% |
| oesophagus | | | | | | | |
| Osteoporosis | 71.77% | 71.94% | 0.77% | 3.67% | 3.65% | −1.38% | 1.76% |
| Other joint disorder | 50.51% | 50.55% | 8.05% | 0.62% | 0.61% | −27.80% | 0.60% |
| Pleural effusion | 55.53% | 55.92% | 7.08% | 0.78% | 0.77% | −2.67% | 0.61% |
| Pneumonia | 52.46% | 53.75% | 52.23% | 1.89% | 1.98% | 71.05% | 1.76% |
| Psoriasis | 65.93% | 69.96% | 25.32% | 2.73% | 3.60% | 59.80% | 1.26% |
| PE +/− DVT | 53.52% | 54.39% | 24.61% | 1.12% | 1.04% | −33.39% | 0.88% |
| Respiratory infection | 53.23% | 55.10% | 58.13% | 2.15% | 2.21% | 22.05% | 1.88% |
| Rheumatoid arthritis | 60.03% | 62.85% | 28.14% | 1.79% | 2.21% | 77.95% | 1.25% |
| Rubella/german measles | 53.95% | 54.71% | 19.12% | 0.74% | 0.82% | 106.25% | 0.68% |
| Sciatica | 52.14% | 53.54% | 65.85% | 1.25% | 1.33% | 65.47% | 1.14% |
| Stroke | 59.06% | 59.80% | 8.11% | 2.14% | 2.27% | 23.21% | 1.58% |
| Tuberculosis (tb) | 51.42% | 53.69% | 160.91% | 0.57% | 0.68% | 307.11% | 0.54% |
| Ulcerative colitis | 57.43% | 60.72% | 44.32% | 0.77% | 0.77% | 4.13% | 0.55% |
| Urinary tract infection/ | 58.20% | 58.19% | −0.17% | 0.76% | 0.75% | −5.71% | 0.58% |
| kidney infection | | | | | | | |
| Varicose veins | 58.09% | 59.84% | 21.72% | 0.71% | 0.76% | 27.42% | 0.52% |
| Whooping cough/pertussis | 52.32% | 51.15% | −50.68% | 0.83% | 0.84% | 12.82% | 0.79% |

^{#}Best AUC highlighted in bold
*Relative increase = (MTL AUC − baseline AUC) − (STL AUC − baseline AUC) × 100%

Identification of important SNPs for MTL by model interpretation

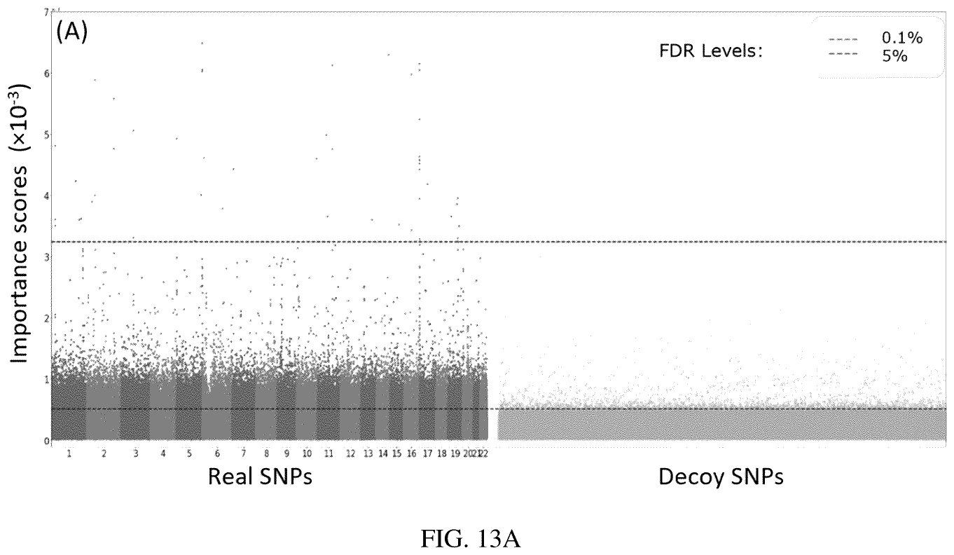

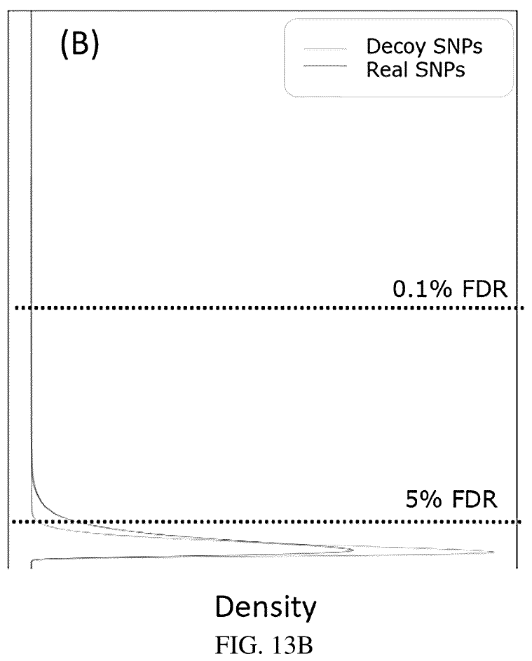

The first-order model-wise LINA interpretation algorithm above was used to identify the important SNPs used by MTL to predict each disease. A pan-cancer MTL model was trained and interpreted using an input whole-genome vector that contained the real SNPs and an equal number of decoy SNPs. FIG. 13 shows the distributions of importance scores for the real SNPs and the decoy SNPs used by the MTL model to predict malignant melanoma. There were 59,350 real SNPs and 3091 decoy SNP with important scores above 0.52×10⁻³, which corresponded to a 5% FDR, because decoy SNPs with random association with the trait cannot be truly important for prediction. At the estimated FDR level of 0.1%, 48 real SNPs and no decoy SNP were identified as important for the MTL model to predict malignant melanoma. Many of these important SNPs have been identified as risk variants for melanoma in previous GWAS studies, including rs12203592 (Gibbs et al., 2017), rs62389423 (Ransohoff et al., 2017), rs4785763 (Bishop et al., 2009), rs4238833 (Bishop et al., 2009), rs10931936 (Landi et al., 2020), rs1126809 (Landi et al., 2020), and Affx-35293625 (Brandes et al., 2021).

Important SNPs in the pan-cancer MTL model were identified for the 17 prevalent cancers at the FDR levels of 0.1% and 5% (Table 15). The number of important SNPs at 0.1% FDR was 29 on average across the 17 cancers with substantial variability. These important SNPs may have strong associations with the traits. At 5% FDR, an average of 36,048 important SNPs were identified for the cancers, suggesting the use of diffused weak association signals across the whole genome by MTL for trait prediction.

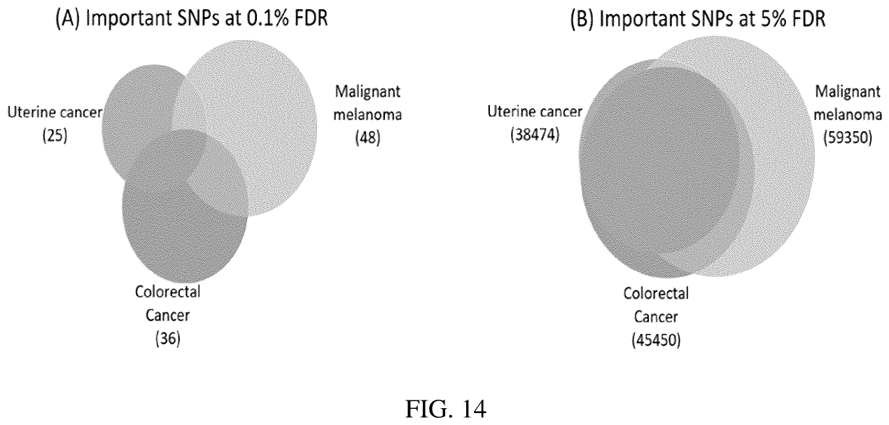

The overlaps among the important SNPs for different diseases were investigated. At 0.1% FDR, only 4 common SNPs were shared among uterine cancer's 25 important SNPs, colorectal cancer's 36 important SNPs, and malignant melanoma's 48 important SNPs (FIG. 14A). The number of important SNPs in the intersection for every pair of diseases at 0.1% FDR were listed in Table 16. The relatively small intersections between different cancers indicated distinct SNP sets with large effect sizes for different diseases. At 5% FDR, there were 21041 common SNPs shared among uterine cancer's 38474 important SNPs, colorectal cancer's 45450 important SNPs, and malignant melanoma's 59350 important SNPs (IG. 14B). Genetic correlations were computed between every pair of cancers based on their importance scores for the SNPs important for one of the diseases or both at 5% FDR (Table 17). The genetic correlations were 0.88 between breast cancer and uterine cancer and 0.89 between lung cancer and lymphoma. Overall, 184 pairs of diseases have positive correlation coefficients between 0.5 and 1.0, 97 pairs have positive correlation coefficients between 0 and 0.5, and only 8 pairs have negative correlation coefficients. This suggested that MTL identified and may have exploited extensive genetic correlations between diseases to achieve a positive knowledge transfer among diseases for PRS estimation.

TABLE 15
Numbers of important SNPs used by pan-cancer
MTL to estimate PRS of prevalent cancers

FDR levels

| Disease | 0.10 | 5.0% |
|---|---|---|
| Malignant melanoma | 48 | 59350 |
| Non-melanoma skin cancer | 132 | 48848 |
| Skin cancer | 106 | 48419 |
| Lung cancer | 4 | 41075 |
| Intrathoracic cancer | 3 | 40392 |
| Colorectal cancer | 36 | 45450 |
| Colon cancer | 22 | 37487 |
| Rectal cancer | 28 | 47904 |
| Bladder cancer | 8 | 37 |
| Uterine cancer | 25 | 38474 |
| Cervical cancer | 5 | 42068 |
| Prostate cancer | 23 | 94 |
| Breast cancer | 34 | 96 |
| Female genital tract cancer | 15 | 37083 |
| Male genital tract cancer | 5 | 40742 |
| Lymphoma | 0 | 43412 |
| Non-hodgkins lymphoma | 4 | 41889 |

Learning many tasks together in a neural network model does not automatically guarantee performance boost for all tasks (Fifty et al., 2021), (Joshi et al., 2019). Negative knowledge transfer can occur between unrelated tasks and, thereby, degrade the performance of a MTL model for these tasks (Bingel and Søgaard, 2017). We did not assume a priori which sets of diseases might be genetically related and could benefit from MTL. By aggregating many diseases together, we discovered positive knowledge transfer for most of the prevalent diseases studied here. The extent of positive knowledge transfer was quantified for each disease based on the gain of predictive performance by MTL relative to STL. For example, malignment melanoma and uterine cancer benefited substantially from parallel training with the other cancers in the pan-cancer MTL, but the extent of positive knowledge transfer to the two cancers was reduced when adding many non-cancer diseases in the pan-disease MTL. The majority of common cancers, including intrathoracic cancer, rectal cancer, and cervical cancer, gained additional performance by scaling MTL from 69 cancers to 362 diseases.

Beneficial transfer learning was also evident for most of the non-cancer common diseases. Consistent observation of increased PRS accuracies for so many diseases provided strong support for the positive knowledge transfer during parallel learning of the genetic risks for complex diseases. To understand how the PRS estimation benefited from MTL, we interpreted a pancancer MTL model and identified important SNPs for each cancer at two empirically estimated FDR levels. Many diseases shared a significant fraction of important SNPs at 5% FDR for their predictions. This suggested a beneficial joint selection of SNPs predictive of multiple diseases. This could be attributed to pleiotropy, wherein a genetic variant may have effects on multiple traits. A meta-analysis of many complex traits' GWAS results estimated 31% of the SNPs and 63% of the genes to be pleiotropic (Watanabe et al., 2019). In addition, the joint feature selection in MTL may be better at filtering out SNPs with random trait associations in the training data than the disease-specific feature selection in STL can.

Data amplification may be a second mechanism for beneficial transfer learning in PRS estimation. Many diseases have an epidemiological correlation. For example, Woo et al. found a 75% greater risk of overall incident cancers after asthma diagnosis in adults (Woo et al., 2021). Pooling the positive cases of multiple diseases together to train a MTL model may increase the effective sample size for learning a shared latent representation predictive of these diseases. Furthermore, many cancers may have some common genetic etiology. Pan-cancer risk variants may elevate the overall risk of individuals for cancers (Rashkin et al., 2020), and some environmental factors may determine the specific site of carcinogenesis. Pooling many cancer cases together may amplify the signal for discovering pan-cancer risk variants. Besides feature selection and data amplification, other mechanisms, such as eavesdropping, representation bias, and regularization (Caruana, 1998), may also contribute to the positive knowledge transfer between diseases for PRS estimation.

Because hard parameter sharing was used in our neural networks from the input layer to the attention layer, the beneficial transfer learning may have produced a latent representation of the genomic data with better generalization for many diseases. Pervasive genetic correlations between diseases allowed MTL to improve the PRS estimation broadly across diseases. While many cross-strait studies have shown the genetic correlation between specific pairs of diseases (Zhuang et al., 2021; Sutton et al., 2022; Wu et al., 2022; Zhou et al., 2022; Adewuyi et al., 2022; Lutz et al., 2020; Byun et al., 2021), our study suggested that various degrees of shared genetic basis may be very prevalent among many complex diseases. Our results highlighted the potential value of holistic association studies between the whole human phenome and the whole human genome for both risk variant discovery and PRS estimation.

Deep neural network improves the estimation of polygenic risk scores for breast cancer

In the present work different computational models for estimating polygenic risk scores (PRS) for breast cancer were compared using genetic variants across the whole genome. A deep neural network (DNN) outperformed established statistical algorithms such as BLUP, BayesA, and LDpred. In a test cohort with 50% prevalence, DNN achieved an area under the receiver operating characteristic curve (AUC) of 67.4% and was able to separate the case population into high- and normal-genetic-risk sub-populations. The PRS generated by DNN in the case population followed a bi-modal distribution composed of two normal distributions with distinctly different means. This suggests that DNN was able to separate the case population into a high-genetic-risk case subpopulation with an average PRS significantly higher than the control population and a normal-genetic-risk case sub-population with an average PRS similar to the control population. This allowed DNN to achieve 18.8% recall at 90% precision in the test cohort with 50% prevalence, which can be extrapolated to 65.4% recall at 20% precision in a general population with 12% prevalence. Interpretation of the DNN model identified interesting variants assigned insignificant p-values by association studies but were important for DNN prediction. These variants may be associated with the phenotype through non-linear relationships or epistatic interactions.

### A Linearizing Neural Network Architecture for Accurate First-Order and Second-Order Interpretations

Although neural networks can yield high predictive performance, the lack of interpretability has hindered the identification of salient features and important feature interactions used for their predictions. This represented a key hurdle for deploying neural networks in many biomedical applications that require interpretability, including predictive genomics. LINA was developed to provide both the first-order and the second-order interpretations on both the instance-wise and the model-wise levels. LINA combines the representational capacity of a deep inner attention neural network with a linearized intermediate representation for model interpretation. In comparison with DeepLIFT, LIME, Grad*Input, and L2X, the first-order interpretation of LINA had better Spearman correlations with the ground-truth importance rankings of features in synthetic datasets. In comparison with NID and GEH, the second-order interpretation results from LINA achieved better precision for the identification of the ground-truth feature interactions in synthetic datasets. These algorithms were further benchmarked using predictive genomics as a real-world application. LINA identified larger numbers of SNPs and salient SNP interactions than the other algorithms at given false discovery rates. The results showed accurate and versatile model interpretation using LINA.

Explainable multi-task learning improves the parallel estimation of polygenic risk scores for many diseases through shared genetic basis

A multi-task learning (MTL) neural network architecture was developed to predict many disease traits of an individual from their whole genome. The model used a shared latent genomic representation, and each trait was predicted from the shared representation via a task-specific hidden layer. This work used the UK Biobank dataset to extract 362 disease traits, including 69 cancer traits and constructed two MTL models-one to predict the 69 cancers and the other to predict all 362 diseases. The MTL models achieved higher predictive performance than single-task learning (STL) models for malignant melanoma and 17 common cancers with prevalence levels higher than 0.5%. The MTL models also showed improved accuracy for predicting 60 noncancer diseases with prevalence levels higher than 0.5%. The study suggested that the performance improvement from transfer learning increased with the number of traits in MTL. The first-order model-wise LINA interpretation algorithm was utilized to identify important SNPs used by Multi-Task Learning (MTL) to predict cancer diseases. A pan-cancer MTL model was trained and interpreted using real and decoy SNPs. At FDR levels of 0.1% and 5%, important SNPs were identified for 17 prevalent cancers, with a higher number of important SNPs identified at 5% FDR. The overlaps among the important SNPs for different diseases were investigated, and small intersections between different cancers were found, indicating distinct SNP sets with large effect sizes for different diseases. At 5% FDR, genetic correlations were computed between every pair of cancers based on their importance scores for the SNPs important for one of the diseases or both. The genetic correlations demonstrated that MTL identified and exploited extensive genetic correlations between diseases to achieve a positive knowledge transfer among diseases for PRS estimation.

In at least one embodiment, the present disclosure is directed to a method of generating polygenic risk scores (PRS) for a plurality of diseases or conditions in a subject, including the steps of (1) collecting a DNA sample from the subject; (2) analyzing the DNA sample for specific single nucleotide polymorphisms (SNPs); (3) obtaining a multi-task learning (MTL) neural network model trained with whole-genome SNP data from a cohort comprising a plurality of phenotypic labels; (4) using the MTL model to (a) identify relationships between the whole-genome SNP data and the plurality of phenotypic labels in the cohort, and (2) calculate PRS for the plurality of diseases or conditions; (5) using the calculated PRS and the SNPs from the DNA sample to determine the subject's risk for developing one or more of the plurality of diseases or conditions; and (6) notifying the subject of the subject's risk for developing the one or more of the plurality of diseases or conditions. Further, when the subject's risk is an increased risk, the method may include the step of administering to the subject a therapeutic, clinical, or preventive action to address the one or more of the plurality of diseases or conditions for which the subject has an increased risk.

### CITED REFERENCES

1. Abravaya, K., J. Huff, R. Marshall, B. Merchant, C. Mullen, G. Schneider, and J. Robinson, 2003: Molecular beacons as diagnostic tools: technology and applications.
2. Adeel, A., M. Gogate, and A. Hussain, 2020: Contextual deep learning-based audiovisual switching for speech enhancement in real-world environments. Information Fusion, 59, 163-170.
3. Adewuyi, E. O., E. K. O'Brien, D. R. Nyholt, T. Porter, and S. M. Laws, 2022: A large-scale genome-wide cross-trait analysis reveals shared genetic architecture between Alzheimer's disease and gastrointestinal tract disorders. Communications Biology, 5 (1), 1-14, https://doi.org/10.1038/s42003-022-03607-2, Nature Publishing Group.
4. Amos, C. I., and Coauthors, 2017: The oncoarray consortium: A network for understanding the genetic architecture of common cancersthe oncoarray and common cancer etiology. Cancer epidemiology, biomarkers & prevention, 26 (1), 126-135.
5. Angermueller, C., T. Pa″rnamaa, L. Parts, and O. Stegle, 2016: Deep learning for computational biology. Molecular systems biology, 12 (7), 878.
6. Badré, A., L. Zhang, W. Muchero, J. C. Reynolds, and C. Pan, 2021: Deep neural network improves the estimation of polygenic risk scores for breast cancer. Journal of Human Genetics, 66 (4), 359-369.
7. Badré, A., and C. Pan, 2022: LINA: A Linearizing Neural Network Architecture for Accurate First-Order and Second-Order Interpretations. IEEE Access, 10, 36166-36176, https://doi.org/10.1109/ACCESS.2022.3163257, conference Name: IEEE Access.
8. Baltres, A., Z. Al Masry, R. Zemouri, S. Valmary-Degano, L. Arnould, N. Zerhouni, and C. Devalland, 2020: Prediction of oncotype dx recurrence score using deep multilayer perceptrons in estrogen receptor-positive, her2-negative breast cancer. Breast Cancer, 27, 1007-1016.
9. Bellot, P., G. de Los Campos, and M. P′erez-Enciso, 2018: Can deep learning improve genomic prediction of complex human traits? Genetics, 210 (3), 809-819.
10. Bengio, Y., and Coauthors, 2009: Learning deep architectures for ai. Foundations and trends® in Machine Learning, 2 (1), 1-127.
11. Bermeitinger., B., T. Hrycej., and S. Handschuh., 2019: Representational capacity of deep neural networks: A computing study. Proceedings of the 11th International Joint Conference on Knowledge Discovery, Knowledge Engineering and Knowledge Management—KDIR,, SciTePress, INSTICC, 532-538, https://doi.org/10.5220/00 08364305320538.
12. Bingel, J., and A. Søgaard, 2017: Identifying beneficial task relations for multi-task learning in deep neural networks. Proceedings of the 15th Conference of the European Chapter of the Association for Computational Linguistics: Volume 2, Short Papers, Association for Computational Linguistics, Valencia, Spain, 164-169, URL https://aclanthology.org/E17-2026.
13. Bishop, D. T., and Coauthors, 2009: Genome-wide association study identifies three loci associated with melanoma risk. Nature Genetics, 41 (8), 920-925, https://doi.org/10.1038/ng.411, URL https://www.nature.com/articles/ng.411, number: 8 Publisher: Nature Publishing Group.
14. Bonferroni, C. E., 1935: Il calcolo delle assicurazioni su gruppi di teste. Studi in onore del professore salvatore ortu carboni, 13-60.
15. Bracher-Smith, M., E. Rees, G. Menzies, J. T. R. Walters, M. C. O'Donovan, M. J. Owen, G. Kirov, and V. Escott-Price, 2022: Machine learning for prediction of schizophrenia using genetic and demographic factors in the UK biobank. Schizophrenia Research, 246, 156-164, https://doi.org/10.1016/j.schres.2022.06.006, URL https://www.sciencedirect.com/science/article/pii/S0920996422002407.
16. Brandes, N., N. Linial, and M. Linial, 2021: Genetic association studies of alterations in protein function expose recessive effects on cancer predisposition. Scientific Reports, 11 (1), 14901, https://doi.org/10.1038/s41598-021-94252-y, URL https://www. nature.com/articles/s41598-021-94252-y, number: 1 Publisher: Nature Publishing Group.
17. Bycroft, C., and Coauthors, 2018: The uk biobank resource with deep phenotyping and genomic data. Nature, 562 (7726), 203-209.
18. Byun, J., and Coauthors, 2021: The Shared Genetic Architectures Between Lung Cancer and Multiple Polygenic Phenotypes in Genome-Wide Association Studies. Cancer Epidemiology, Biomarkers & Prevention, 30 (6), 1156-1164, https://doi.org/10.115 8/1055-9965.EPI-20-1635.
19. Cesaratto, L., and Coauthors, 2016: Bnc2 is a putative tumor suppressor gene in highgrade serous ovarian carcinoma and impacts cell survival after oxidative stress. Cell death & disease, 7 (9), e2374-e2374.
20. Chan, C. H. T., and Coauthors, 2018: Evaluation of three polygenic risk score models for the prediction of breast cancer risk in singapore chinese. Oncotarget, 9 (16), 12796.
21. Chang, C. C., C. C. Chow, L. C. Tellier, S. Vattikuti, S. M. Purcell, and J. J. Lee, 2015: Second-generation plink: rising to the challenge of larger and richer datasets. Gigascience, 4 (1), s13742-015.
22. Chen, J., L. Song, M. Wainwright, and M. Jordan, 2018: Learning to explain: An information-theoretic perspective on model interpretation. International Conference on Machine Learning, PMLR, 883-892.
23. Clark, S. A., B. P. Kinghorn, J. M. Hickey, and J. H. van der Werf, 2013: The effect of genomic information on optimal contribution selection in livestock breeding programs. Genetics selection evolution, 45, 1-8.
24. Collobert, R., and J. Weston, 2008: A unified architecture for natural language processing: Deep neural networks with multitask learning. Proceedings of the 25th international conference on Machine learning, 160-167.
25. Consortium,. G. P., and Coauthors, 2015: A global reference for human genetic variation. Nature, 526 (7571), 68.
26. Cordell, H. J., 2002: Epistasis: what it means, what it doesn't mean, and statistical methods to detect it in humans. Human molecular genetics, 11 (20), 2463-2468.
27. Cudic, M., H. Baweja, T. Parhar, and S. T. Nuske, 2018: Prediction of Sorghum bicolor genotype from in-situ images using autoencoder-identified snps. 2018 17th IEEE International Conference on Machine Learning and Applications (ICMLA), IEEE, 23-31.
28. Cui, T., P. Marttinen, and S. Kaski, 2019: Learning global pairwise interactions with bayesian neural networks. arXiv preprint arXiv: 1901.08361.
29. Dandl, S., C. Molnar, M. Binder, and B. Bischl, 2020: Multi-objective counterfactual explanations. Parallel Problem Solving from Nature-PPSN XVI: 16th International Conference, PPSN 2020, Leiden, The Netherlands, Sep. 5-9, 2020, Proceedings, Part I, Springer, 448-469.
30. Dayem Ullah, A. Z., J. Oscanoa, J. Wang, A. Nagano, N. R. Lemoine, and C. Chelala, 2018: Snpnexus: assessing the functional relevance of genetic variation to facilitate the promise of precision medicine. Nucleic acids research, 46 (W1), W109-W113.
31. De, R., W. S. Bush, and J. H. Moore, 2014: Bioinformatics challenges in genome-wide association studies (gwas). Clinical Bioinformatics, 63-81.
32. DeBoever, C., Y. Tanigawa, M. Aguirre, G. McInnes, A. Lavertu, and M. A. Rivas, 2020: Assessing Digital Phenotyping to Enhance Genetic Studies of Human Diseases. The American Journal of Human Genetics 106 (5), 611-622, https://doi.org/10.1 016/j.ajhg.2020.03.007, URL https://www.cell.com/ajhg/abstract/S0002-9297 (20) 3 0083-5, publisher: Elsevier.
33. Deng, L., G. Hinton, and B. Kingsbury, 2013: New types of deep neural network learning for speech recognition and related applications: An overview. 2013 IEEE international conference on acoustics, speech and signal processing, IEEE, 8599-8603.
34. Do, D. T., and N. Q. K. Le, 2020: Using extreme gradient boosting to identify origin of replication in Saccharomyces cerevisiae via hybrid features. Genomics, 112 (3), 2445-2451.
35. Dudbridge, F., 2013: Power and predictive accuracy of polygenic risk scores. PLoS genetics, 9 (3), e1003348.
36. El Naqa, I., and M. J. Murphy, 2015: What is machine learning? Springer. Elgart, M., and Coauthors, 2022: Non-linear machine learning models incorporating SNPs and PRS improve polygenic prediction in diverse human populations. Communications Biology, 5 (1), 1-12, https://doi.org/10.1038/s42003-022-03812-z, Nature Publishing Group.
37. Fergus, P., C. C. Montanez, B. Abdulaimma, P. Lisboa, C. Chalmers, and B. Pineles, 2018: Utilizing deep learning and genome wide association studies for epistatic-driven preterm birth classification in african-american women. IEEE/ACM transactions on computational biology and bioinformatics, 17 (2), 668-678.
38. Fernald, K., and M. Kurokawa, 2013: Evading apoptosis in cancer. Trends in cell biology, 23 (12), 620-633.
39. Fifty, C., E. Amid, Z. Zhao, T. Yu, R. Anil, and C. Finn, 2021: Efficiently Identifying Task Groupings for Multi-Task Learning. Advances in Neural Information Processing Systems, Curran Associates, Inc., Vol. 34, 27503-27516, URL https://proceedings. neurips.cc/paper/2021/hash/e77910ebb93b511588557806310f78f1-Abstract.html.
40. Friedman, J. H., 2001: Greedy function approximation: a gradient boosting machine. Annals of statistics, 1189-1232.
41. Fukushima, K., 1975: Cognitron: A self-organizing multilayered neural network. Biological cybernetics, 20 (3-4), 121-136.
42. Gao, M., Y. Quan, X.-H. Zhou, and H.-Y. Zhang, 2019: Phewas-based systems genetics methods for anti-breast cancer drug discovery. Genes, 10 (2), 154.
43. Ge, T., C.-Y. Chen, Y. Ni, Y.-C. A. Feng, and J. W. Smoller, 2019: Polygenic prediction via bayesian regression and continuous shrinkage priors. Nature communications, 10 (1), 1776.
44. Ge, T., and Coauthors, 2022: Development and validation of a trans-ancestry polygenic risk score for type 2 diabetes in diverse populations. Genome Medicine, 14 (1), 70, https://doi.org/10.1186/s13073-022-01074-2, URL https://doi.org/10.1186/s13073-022-01074-2.
45. Gibbs, D., and Coauthors, 2017: Functional melanoma-risk variant IRF4 rs12203592 associated with Breslow thickness: a pooled international study of primary melanomas. British Journal of Dermatology, 177 (5), e180-e182, https://doi.org/10.1111/bjd.15784.
46. Girshick, R., 2015: Fast r-cnn. Proceedings of the IEEE international conference on computer vision, 1440-1448.
47. Gola, D., J. Erdmann, B. Mu “ller-Myhsok, H. Schunkert, and I. R. K″onig, 2020: Polygenic risk scores outperform machine learning methods in predicting coronary artery disease status. Genetic Epidemiology, 44 (2), 125-138, https://doi.org/10.1002/ge pi.22279,.
48. Gon calves, E., and Coauthors, 2020: Drug mechanism-of-action discovery through the integration of pharmacological and crispr screens. Molecular Systems Biology, 16 (7), e9405.
49. Hastie, T., S. Rosset, J. Zhu, and H. Zou, 2009: Multi-class adaboost. Statistics and its Interface, 2 (3), 349-360.
50. Henderson, C. R., 1975: Best linear unbiased estimation and prediction under a selection model. Biometrics, 423-447.
51. Ho, D. S. W., W. Schierding, M. Wake, R. Saffery, and J. O'Sullivan, 2019: Machine learning snp based prediction for precision medicine. Frontiers in genetics, 10, 267.
52. Ho Thanh Lam, L., N. H. Le, L. Van Tuan, H. Tran Ban, T. Nguyen Khanh Hung, N. T. K. Nguyen, L. Huu Dang, and N. Q. K. Le, 2020: Machine learning model for identifying antioxidant proteins using features calculated from primary sequences. Biology, 9 (10), 325.
53. Hsieh, Y.-C., and Coauthors, 2017: A polygenic risk score for breast cancer risk in a taiwanese population. Breast cancer research and treatment, 163, 131-138.
54. Ioffe, S., and C. Szegedy, 2015: Batch normalization: Accelerating deep network training by reducing internal covariate shift. ArXiv, abs/1502.03167.
55. Jobs, M., 2001: Robust and accurate single nucleotide polymorphism genotyping by dynamic allele specific hybridization (dash). SNP 2000: Third International Meeting on Single Nucleotide polymorphism and Complex Genome Analysis, Taos, New Mexico, USA, 2001.
56. Joshi, A., S. Karimi, R. Sparks, C. Paris, and C. R. MacIntyre, 2019: Does Multi-Task Learning Always Help?: An Evaluation on Health Informatics. Proceedings of The 17th Annual Workshop of the Australasian Language Technology Association, Australasian Language Technology Association, Sydney, Australia, 151-158, URL https://aclanthology.org/U19-1020.
57. Khera, A. V., and Coauthors, 2018: Genome-wide polygenic scores for common diseases identify individuals with risk equivalent to monogenic mutations. Nature genetics, 50 (9), 1219-1224.
58. Kingma, D. P., and J. Ba, 2014a: Adam: A method for stochastic optimization. arXiv preprint arXiv: 1412.6980.
59. Kingma, D. P., and J. Ba, 2014b: Adam: A method for stochastic optimization. CoRR, abs/1412.6980.
60. Kolch, W., M. Halasz, M. Granovskaya, and B. N. Kholodenko, 2015: The dynamic control of signal transduction networks in cancer cells. Nature Reviews Cancer, 15 (9), 515-527.
61. Koppe, G., A. Meyer-Lindenberg, and D. Durstewitz, 2021: Deep learning for small and big data in psychiatry. Neuropsychopharmacology, 46 (1), 176-190.
62. Landi, M. T., and Coauthors, 2020: Genome-wide association meta-analyses combining multiple risk phenotypes provide insights into the genetic architecture of cutaneous melanoma susceptibility. Nature Genetics, 52 (5), 494-504, https://doi.org/10.103 8/s41588-020-0611-8, Nature Publishing Group.
63. Lawson, D. J., and Coauthors, 2020: Is population structure in the genetic biobank era irrelevant, a challenge, or an opportunity? Human Genetics, 139, 23-41.
64. LeBlanc, M., and C. Kooperberg, 2010: Boosting predictions of treatment success. Proceedings of the National Academy of Sciences, 107 (31), 13559-13560.
65. LeCun, Y., L. Bottou, Y. Bengio, and P. Haffner, 1998: Gradient-based learning applied to document recognition. Proceedings of the IEEE, 86 (11), 2278-2324.
66. Lee, J.-Y., and Coauthors, 2009: Candidate gene approach evaluates association between innate immunity genes and breast cancer risk in korean women. Carcinogenesis, 30 (9), 1528-1531.
67. Li, X., Z. Zou, J. Tang, Y. Zheng, Y. Liu, Y. Luo, Q. Liu, and Y. Wang, 2019: Nos1 upregulates abcg2 expression contributing to ddp chemoresistance in ovarian cancer cells. Oncology letters, 17 (2), 1595-1602.
68. Liu, P., X. Qiu, and X. Huang, 2016: Recurrent neural network for text classification with multi-task learning. Proceedings of the Twenty-Fifth International Joint Conference on Artificial Intelligence, AAAI Press, New York, New York, USA, 2873-2879, IJCAI′16.
69. Lu, L., Y. Shin, Y. Su, and G. E. Karniadakis, 2019: Dying relu and initialization: Theory and numerical examples. arXiv preprint arXiv: 1903.06733.
70. Lundberg, S. M., and S.-I. Lee, 2017: A unified approach to interpreting model predictions. Advances in neural information processing systems, 30.
71. Lutz, M. W., D. Sprague, J. Barrera, and O. Chiba-Falek, 2020: Shared genetic etiology underlying Alzheimer's disease and major depressive disorder. Translational Psychiatry, 10 (1), 1-14, https://doi.org/10.1038/s41398-020-0769-y, Nature Publishing Group.
72. Maas, A. L., A. Y. Hannun, A. Y. Ng, and Coauthors, 2013: Rectifier nonlinearities improve neural network acoustic models. Proc. icml, Atlanta, Georgia, USA, Vol. 30, 3.
73. Maier, R., and Coauthors, 2015: Joint analysis of psychiatric disorders increases accuracy of risk prediction for schizophrenia, bipolar disorder, and major depressive disorder. The American Journal of Human Genetics, 96 (2), 283-294.
74. Mailman, M. D., and Coauthors, 2007: The ncbi dbgap database of genotypes and phenotypes. Nature genetics, 39 (10), 1181-1186.
75. Mao, Q., and J. D. Unadkat, 2015: Role of the breast cancer resistance protein (bcrp/abcg2) in drug transport—an update. The AAPS journal, 17, 65-82.
76. Marchini, J., L. R. Cardon, M. S. Phillips, and P. Donnelly, 2004: The effects of human population structure on large genetic association studies. Nature genetics, 36 (5), 512-517.
77. Mavaddat, N., and Coauthors, 2019: Polygenic risk scores for prediction of breast cancer and breast cancer subtypes. The American Journal of Human Genetics, 104 (1), 21-34.
78. Meuwissen, T. H., B. J. Hayes, and M. Goddard, 2001: Prediction of total genetic value using genome-wide dense marker maps. genetics, 157 (4), 1819-1829.
79. Michailidou, K., and Coauthors, 2017: Association analysis identifies 65 new breast cancer risk loci. Nature, 551 (7678), 92-94.
80. Miller, T., 2019: Explanation in artificial intelligence: Insights from the social sciences. Artificial intelligence, 267, 1-38.
81. Ma′rquez-Luna, C., S. Gazal, P.-R. Loh, S. S. Kim, N. Furlotte, A. Auton, and A. L. Price, 2021: Incorporating functional priors improves polygenic prediction accuracy in UK Biobank and 23andMe data sets. Nature Communications, 12 (1), 6052, https://doi.org/10.1038/s41467-021-25171-9, Nature Publishing Group.
82. Nelson, H. D., K. Tyne, A. Naik, C. Bougatsos, B. K. Chan, and L. Humphrey, 2009: Screening for breast cancer: an update for the us preventive services task force. Annals of internal medicine, 151 (10), 727-737.
83. NIH, 2012: Cancer of the breast (female)-cancer stat facts. URL https://seer.cance r.gov/statfacts/html/breast.html.
84. Novembre, J., and Coauthors, 2008: Genes mirror geography within europe. Nature, 456 (7218), 98-101.
85. Oeffinger, K. C., and Coauthors, 2015: Breast cancer screening for women at average risk: 2015 guideline update from the american cancer society. Jama, 314 (15), 1599-1614.
86. O'Connor, M. J., 2015: Targeting the dna damage response in cancer. Molecular cell, 60 (4), 547-560.
87. Pace, R. K., and R. Barry, 1997: Sparse spatial autoregressions. Statistics & Probability Letters, 33 (3), 291-297.
88. Phillips, P. C., 2008: Epistasis—the essential role of gene interactions in the structure and evolution of genetic systems. Nature Reviews Genetics, 9 (11), 855-867.
89. Priv′e, F., J. Arbel, and B. J. Vilhj′almsson, 2020: LDpred2: better, faster, stronger. Bioinformatics, 36 (22-23), 5424-5431, https://doi.org/10.1093/bioinformatics/b taa1029.
90. Purcell, S., and Coauthors, 2007: Plink: a tool set for whole-genome association and population-based linkage analyses. The American journal of human genetics, 81 (3), 559-575.
91. Purcell Shaun, S. J. V. P. O. M. C . . . . S. P. F . . . . S. P . . . , Wray Naomi, and Coauthors, 2009: Common polygenic variation contributes to risk of schizophrenia and bipolar disorder. Nature, 460 (7256), 748-752.
92. Ransohoff, K. J., and Coauthors, 2017: Two-stage genome-wide association study identifies a novel susceptibility locus associated with melanoma. Oncotarget, 8 (11), 17586-17592, https://doi.org/10.18632/oncotarget.15230, URL https://www.onco target.com/article/15230/text/, publisher: Impact Journals.
93. Rashkin, S. R., and Coauthors, 2020: Pan-cancer study detects genetic risk variants and shared genetic basis in two large cohorts. Nature Communications, 11 (1), 4423, https://doi.org/10.1038/s41467-020-18246-6, Nature Publishing Group.
94. Ribeiro, M. T., S. Singh, and C. Guestrin, 2016: “Why should i trust you?” explaining the predictions of any classifier. Proceedings of the 22nd ACM SIGKDD international conference on knowledge discovery and data mining, 1135-1144.
95. Rivandi, M., J. W. Martens, and A. Hollestelle, 2018: Elucidating the underlying functional mechanisms of breast cancer susceptibility through post-gwas analyses. Frontiers in genetics, 9, 280.
96. Romualdo Cardoso, S., A. Gillespie, S. Haider, and O. Fletcher, 2022: Functional annotation of breast cancer risk loci: current progress and future directions. British Journal of Cancer, 126 (7), 981-993.
97. Rosenblatt, F., 1958: The perceptron: a probabilistic model for information storage and organization in the brain. Psychological review, 65 (6), 386.
98. Rumelhart, D. E., G. E. Hinton, and R. J. Williams, 1986: Learning representations by back-propagating errors. nature, 323 (6088), 533-536.
99. Schmidhuber, J., 2015: Deep learning in neural networks: An overview. Neural networks, 61, 85-117.
100. Scott, R. A., and Coauthors, 2017: An expanded genome-wide association study of type 2 diabetes in europeans. Diabetes, 66 (11), 2888-2902.
101. Shaker, O. G., and M. A. Senousy, 2019: Association of snp-snp interactions between rankl, opg, chi311, and vdr genes with breast cancer risk in egyptian women. Clinical Breast Cancer, 19 (1), e220-e238.
102. Shrikumar, A., P. Greenside, and A. Kundaje, 2017: Learning important features through propagating activation differences. International conference on machine learning, PMLR, 3145-3153.
103. Simonyan, K., A. Vedaldi, and A. Zisserman, 2014: Deep inside convolutional networks: Visualising image classification models and saliency maps. 2nd International Conference on Learning Representations, ICLR 2014, Banff, AB, Canada, Apr. 14-16, 2014, Workshop Track Proceedings, Y. Bengio, and Y. LeCun, Eds., URL http://arxiv.org/abs/1312.6034.
104. Sorokina, D., R. Caruana, and M. Riedewald, 2007: Additive groves of regression trees. Machine Learning: ECML 2007: 18th European Conference on Machine Learning, Warsaw, Poland, Sep. 17-21, 2007. Proceedings 18, Springer, 323-334.
104. Speed, D., and D. J. Balding, 2014: Multiblup: improved snp-based prediction for complex traits. Genome research, 24 (9), 1550-1557.
105. Srivastava, N., G. E. Hinton, A. Krizhevsky, I. Sutskever, and R. Salakhutdinov, 2014: Dropout: a simple way to prevent neural networks from overfitting. J. Mach. Learn. Res., 15, 1929-1958.
106. Standley, T., A. R. Zamir, D. Chen, L. Guibas, J. Malik, and S. Savarese, 2019: Which Tasks Should Be Learned Together in Multi-task Learning? URL https://openreview.net/forum?id=HJITpCEKvS.
107. Sutton, M., P.-E. Sugier, T. Truong, and B. Liquet, 2022: Leveraging pleiotropic association using sparse group variable selection in genomics data. BMC Medical Research Methodology, 22 (1), 9, https://doi.org/10.1186/s12874-021-01491-8.
108. Thissen, J. B., and Coauthors, 2019: Axiom microbiome array, the next generation microarray for high-throughput pathogen and microbiome analysis. PLoS One, 14 (2), e0212045.
109. Thomas, M., and Coauthors, 2020: Genome-wide Modeling of Polygenic Risk Score in Colorectal Cancer Risk. The American Journal of Human Genetics, 107 (3), 432-444, https://doi.org/10.1016/j.ajhg.2020.07.006, URL https://www.sciencedirect.co m/science/article/pii/S0002929720302366.
110. Tian, H., S.-C. Chen, and M.-L. Shyu, 2020: Evolutionary programming based deep learning feature selection and network construction for visual data classification. Information systems frontiers, 22, 1053-1066.
111. Tinholt, M., and Coauthors, 2014: Increased coagulation activity and genetic polymorphisms in the f5, f10 and epcr genes are associated with breast cancer: a case-control study. Bmc Cancer, 14, 1-11.
112. Tsang, M., D. Cheng, and Y. Liu, 2017: Detecting statistical interactions from neural network weights. International Conference on Machine Learning.
113. Tsuboi, M., and Coauthors, 2019: Prognostic significance of gad1 overexpression in patients with resected lung adenocarcinoma. Cancer medicine, 8 (9), 4189-4199.
114. Uffelmann, E., and Coauthors, 2021: Genome-wide association studies. Nature Reviews Methods Primers, 1 (1), 59.
115. van de Haar, J., S. Canisius, K. Y. Michael, E. E. Voest, L. F. Wessels, and T. Ideker, 2019: Identifying epistasis in cancer genomes: a delicate affair. Cell, 177 (6), 1375-1383.
116. Velasco-Ruiz, A., and Coauthors, 2021: Polrmt as a novel susceptibility gene for cardiotoxicity in epirubicin treatment of breast cancer patients. Pharmaceutics, 13 (11), 1942.
117. Vilhja′Imsson, B. J., and Coauthors, 2015: Modeling linkage disequilibrium increases accuracy of polygenic risk scores. The american journal of human genetics, 97 (4), 576-592.
118. Wang, L., J. Ingle, and R. Weinshilboum, 2018: Pharmacogenomic discovery to function and mechanism: breast cancer as a case study. Clinical Pharmacology & Therapeutics, 103 (2), 243-252.
119. Watanabe, K., and Coauthors, 2019: A global overview of pleiotropy and genetic architecture in complex traits. Nature Genetics, 51 (9), 1339-1348, https://doi.org/10.1038/s41588-019-0481-0, Nature Publishing Group.
120. Wei, Z., and Coauthors, 2009: From disease association to risk assessment: an optimistic view from genome-wide association studies on type 1 diabetes. PLoS genetics, 5 (10), e1000678.
121. Wen, W., and Coauthors, 2016: Prediction of breast cancer risk based on common genetic variants in women of east asian ancestry. Breast Cancer Research, 18 (1), 1-8.
122. Whittaker, A. J., I. Royzman, and T. L. Orr-Weaver, 2000a: Drosophila double parked: a conserved, essential replication protein that colocalizes with the origin recognition complex and links dna replication with mitosis and the down-regulation of s phase transcripts. Genes & development, 14 (14), 1765-1776.
123. Whittaker, A. J., I. Royzman, and T. L. Orr-Weaver, 2000b: Drosophila Double parked: a conserved, essential replication protein that colocalizes with the origin recognition complex and links DNA replication with mitosis and the down-regulation of S phase transcripts. Genes & Development, 14 (14), 1765-1776, https://doi.org/10.1101/gad.14.14.1765, URL http://genesdev.cshlp.org/content/14/14/1765, company: Cold Spring Harbor Laboratory Press.
124. Woo, A., S. W. Lee, H. Y. Koh, M. A. Kim, M. Y. Han, and D. K. Yon, 2021: Incidence of cancer after asthma development: 2 independent population-based cohort studies. Journal of Allergy and Clinical Immunology, 147 (1), 135-143, https://doi.org/10.1016/j.jaci.2020.04.041, publisher: Elsevier.
125. Wu, L., and Coauthors, 2016: A genome-wide association study identifies wtl variant with better response to 5-fluorouracil, pirarubicin and cyclophosphamide neoadjuvant chemotherapy in breast cancer patients. Oncotarget, 7 (4), 5042.
126. Wu, X., and Coauthors, 2022: Investigating the shared genetic architecture of uterine leiomyoma and breast cancer: A genome-wide cross-trait analysis. The American Journal of Human Genetics, 109 (7), 1272-1285, https://doi.org/10.1016/j.ajhg.2 022.05.015, URL https://www.cell.com/ajhg/abstract/S0002-9297 (22) 00253-1, publisher: Elsevier.
127. Xu, B., N. Wang, T. Chen, and M. Li, 2015: Empirical evaluation of rectified activations in convolutional network. arXiv preprint arXiv: 1505.00853.
128. Ye, Y., X. Chen, J. Han, W. Jiang, P. Natarajan, and H. Zhao, 2021: Interactions Between Enhanced Polygenic Risk Scores and Lifestyle for Cardiovascular Disease, Diabetes, and Lipid Levels. Circulation: Genomic and Precision Medicine, 14 (1), e003128, https://doi.org/10.1161/CIRCGEN.120.003128, publisher: American Heart Association.
129. Yin, B., M. Balvert, R. A. van der Spek, B. E. Dutilh, S. Boht′e, J. Veldink, and A. Schonhuth, 2019: Using the structure of genome data in the design of deep neural networks for predicting amyotrophic lateral sclerosis from genotype. Bioinformatics, 35 (14), i538-i547.
130. Young, T., D. Hazarika, S. Poria, and E. Cambria, 2018: Recent trends in deep learning based natural language processing. ieee Computational intelligenCe magazine, 13 (3), 55-75.
131. Zhang, Y., and Q. Yang, 2018: An overview of multi-task learning. National Science Review, 5 (1), 30-43.
132. Zhao, X., J. Li, Z. Liu, and S. Powers, 2021: Combinatorial crispr/cas9 screening reveals epistatic networks of interacting tumor suppressor genes and therapeutic targets in human breast cancer. Cancer Research.
133. Zhou, Y., Z.-S. Liang, Y. Jin, J. Ding, T. Huang, J. H. Moore, Z.-J. Zheng, and J. Huang, 2022: Shared Genetic Architecture and Causal Relationship Between Asthma and Cardiovascular Diseases: A Large-Scale Cross-Trait Analysis. Frontiers in Genetics, 12, URL https://www.frontiersin.org/articles/10.3389/fgene.202 1.775591.
134. Zhuang, Z., M. Yao, J. Y. Y. Wong, Z. Liu, and T. Huang, 2021: Shared genetic etiology and causality between body fat percentage and cardiovascular diseases: a largescale genome-wide cross-trait analysis. BMC Medicine, 19 (1), 100, https://doi.org/10.1186/s12916-021-01972-z.

1. A method of determining a polygenic risk score of a test set of genome-wide single nucleotide polymorphisms (SNPs) for a plurality of diseases or conditions, the method comprising the steps of:
obtaining whole-genome SNP data from a population of at least 50,000 subjects having a known disease outcome and an equal number of decoy SNPs and disease data of the population of subjects, wherein at least 50 different disease outcomes are represented across the population of subjects;
preprocessing the whole-genome SNP data by labeling each SNP as either homozygous with minor allele, heterozygous allele, or homozygous with the dominant allele and preprocessing the decoy SNP data by randomly labeling each decoy SNP as either minor allele, heterozygous allele at the same frequency as in the whole-genome SNP data, or homozygous with the dominant allele to obtain a whole-genome vector; and preprocessing the known disease outcomes to obtain a whole-phenome vector;
training a multi-task learning (MTL) neural network learning model by forming a training set sample by taking the whole-genome vector as the input data and the whole-phenome vector as the output data, using the training set to train the MTL neural network learning model, obtaining optimal parameters of the MTL neural network learning model after multiple rounds of training, and then obtaining a trained model;
inputting the genome-wide SNPs of the test set into the trained model and outputting a predicted polygenic risk score.

2. The method of claim 1, wherein the test set of genome-wide SNPs are the genome-wide SNPs for a single subject.

3. The method of claim 2, wherein the genome-wide SNPs for a single subject are determined by collecting a DNA sample from the single subject and analyzing the DNA sample for genome-wide SNPs.

4. The method of claim 2, wherein the predicted polygenic risk score identifies the single subject's risk for developing one or more of the plurality of diseases or conditions.

5. The method of claim 4, wherein when the subject's risk is an increased risk, administering to the subject a therapeutic, clinical, or preventive action to address the one or more of the plurality of diseases or conditions for which the subject has an increased risk.

6. The method of claim 4, further comprising notifying the single subject of the single subject's risk for developing one or more of the plurality of diseases or conditions.

7. The method of claim 1, wherein the known disease outcomes comprise at least 50 cancer types.

8. The method of claim 1, wherein the known disease outcomes comprise at least 50 cancer types and at least 100 non-cancer diseases.

9. A computer-implemented method of training a multi-task learning (MTL) deep neural network for estimating a polygenic risk score for any one of at least 15 diseases, the method comprising:
collecting (i) a first set of SNPs from at least 50,000 subjects with a known disease outcome from a database and an equal number of decoy SNPs; and (ii) a second set of SNPs from at least 50,000 other subjects with a known disease outcome from a database an equal number of decoy SNPs, wherein at least 50 different disease outcomes are represented across the population of subjects;
encoding, independently, the first set of SNPs and the second set of SNPs by:
labeling each subject based on the known disease outcome for the subject, and
labeling each SNP in each subject as either homozygous with minor allele,
heterozygous allele, or homozygous with the dominant allele;
randomly labeling each decoy SNP as either homozygous with minor allele,
heterozygous allele, or homozygous with the dominant allele;
optionally applying one or more filter to the first encoded set to create a first modified set of SNPs;
training the multi-task learning deep neural network using the first encoded set of SNPs or the first modified set of SNPs; and
validating the multi-task learning deep neural network using the second encoded set of SNPs.

10. The method of claim 9, wherein the filter comprises a p-value threshold.

11. The method of claim 9, wherein the SNPs are genome-wide.

12. The method of claim 9, wherein the SNPs are representative of at least 22 chromosomes.

13. The method of claim 9, wherein both the first set of SNPs and the second set of SNPs comprise the same at least 100,000 SNPs.

14. The method of claim 9, wherein at least 50 cancers are represented in the disease outcomes of the population of subjects.

15. The method of claim 9, wherein at least 100 disease outcomes are represented in the population of subjects.

16. A method of determining an importance score for a SNP or set of SNPs for a plurality of diseases or conditions, the method comprising the steps of:
obtaining whole-genome SNP data from a population of at least 50,000 subjects having a known disease outcome and an equal number of decoy SNPs and disease data of the population of subjects, wherein at least 50 different disease outcomes are represented across the population of subjects;
preprocessing the whole-genome SNP data by labeling each SNP as either homozygous with minor allele, heterozygous allele, or homozygous with the dominant allele and preprocessing the decoy SNP data by randomly labeling each decoy SNP as either minor allele, heterozygous allele at the same frequency as in the whole-genome SNP data, or homozygous with the dominant allele to obtain a whole-genome vector; and preprocessing the known disease outcomes to obtain a whole-phenome vector;
training a multi-task learning (MTL) neural network learning model by forming a training set sample by taking the whole-genome vector as the input data and the whole-phenome vector as the output data, using the training set to train the MTL neural network learning model, obtaining optimal parameters of the MTL neural network learning model after multiple rounds of training, and then obtaining a trained model;
inputting the SNPs of a synthetic genomic vector into the trained model and finding the importance score of each SNP for prediction of each disease based on the Jacobian of the genotypes of the SNPs with respect to the PRS of each disease and filtering the SNPs based on their importance scores to reach a 0.1% FDR, which is computed based on the proportion of the decoy SNPs out of the real SNPs.

17. A method of interpreting the trained multi-task learning (MTL) deep neural network of claim 9, the method comprising finding the importance score of each SNP for prediction of each disease based on the Jacobian of the genotypes of the SNPs with respect to the PRS of each disease and filtering the SNPs based on their importance scores to reach a 0.1% FDR, which is computed based on the proportion of the decoy SNPs out of the real SNPs.

18. A method for assessing a disease outcome in a subject, the method comprising:
obtaining data for a set of SNPs in the subject; and
determining a machine-trained assessment of the disease outcome in the subject using the trained neural network-based nonlinear classifier of claim 9,
wherein the output of the trained neural network is subsequently used for an assessment of a disease outcome.

19. A system comprising:
a processor; and
a memory having instructions thereon, wherein the instructions when executed by the processor, cause the processor to:
obtain data for a set of SNPs in a subject; and
determine an output indicating the disease outcome in the subject, using an output of a trained neural network, wherein the trained neural network comprises a neural network trained using sets of SNPs from a population of at least 50,000 subjects each with a known disease outcome from a database, wherein at least 50 different disease outcomes are represented across the population of subjects,
wherein the output of the trained neural network is subsequently used for an assessment of a disease outcome.

20. A non-transitory computer readable medium having instructions stored thereon, wherein execution of the instructions causes a processor to:
obtain data for a set of SNPs in a subject; and
determine an output indicating the disease outcome in the subject, using an output of a trained neural network, wherein the trained neural network comprises a neural network trained using sets of SNPs from a population of at least 50,000 subjects each with a known disease outcome from a database, wherein at least 50 different disease outcomes are represented across the population of subjects,
wherein the output of the trained neural network is subsequently used for an assessment of a disease outcome.
